(19) United States
(12) Patent Application Publication
(10) Pub. No.: US 2026/0365696 A1
(43) Pub. Date: Nov. 19, 2026

(54) INHIBITORS OF PTP1B / NCK1 INTERACTION AND METHODS OF MAKING AND USING SAME

(71) Applicant: Radella Pharmaceuticals LLC, Wilmington, DE (US)

(72) Inventors: Mirela Delibegovic, Aberdeen (GB); Sergio Dall'Angelo, Aberdeen (GB); Meghan Marie Hamer, Aberdeen (GB); Ruta Dekeryte, Aberdeen (GB); Andrew Robert McEwan, Aberdeen (GB); lain Robert Greig, Aberdeen (GB)

(21) Appl. No.: 19/158,827

(22) PCT Filed: Feb. 22, 2024
(86) PCT No.: PCT/US2024/016944

§ 371 Date: Aug. 21, 2025

Related U.S. Application Data
(60) Provisional application No. 63/486,457, filed on Feb. 22, 2023.

(51) Int. Cl. C07K 7/06; A61K 38/00; A61P 3/04; A61P 3/10; C07K 7/08

(52) U.S. Cl. CPC C07K 7/06; A61P 3/04; A61P 3/10; C07K 7/08; A61K 38/00

(57) ABSTRACT

The invention relates generally to synthetic peptides which bind NCK1. The invention further relates to methods of using the peptides for inhibiting an interaction between NCK1 and PTP1B, and for treating a disease or disorder associated with insulin resistance, including type 2 diabetes and obesity.

### CROSS REFERENCE TO RELATED APPLICATIONS

This application claims the benefit of and priority to U.S. Provisional Application No. 63/486,457, filed Feb. 22, 2023, the entire disclosure of which is hereby incorporated by reference in its entirely for all purposes.

### SEQUENCE LISTING

This application contains a Sequence Listing which has been submitted electronically in XML format and is hereby incorporated by reference in its entirety. The XML copy, created on Feb. 19, 2024, is named CGP-001WO_SL.xml and is 252 kilobytes in size.

### FIELD OF THE DISCLOSURE

The disclosure relates generally to synthetic peptides which bind NCK1 and their use in treating diseases and disorders, including obesity and Type 2 Diabetes (T2D).

### BACKGROUND

Diabetes is a chronic metabolic disease involving the production and/or activity of insulin. It is estimated that more than 500 million people around the world are living with diabetes, and this number is expected to continue to increase. In the United States alone, 140 million adults have diabetes or prediabetes, mostly linked to excessive body weight and sedentary lifestyle. Type 2 diabetes (T2D; i.e., Type 2 diabetes mellitus) is the most common form of diabetes, accounting for approximately 90% of diabetes cases (Ong et al. (2023) LANCET 402(10397): 203-34). T2D is characterized by hyperglycemia resulting from insulin resistance. There is no approved cure for T2D, and treatment for the disease can require a lifetime of continuous medication. T2D is a progressive condition and thus, treatment typically must increase over time to keep blood glucose levels within the recommended range. Complications from T2D can have a considerable impact on health, resulting in damage to organs throughout the body. T2D is also a significant cause of blindness, kidney failure, heart attacks, stroke, and lower limb amputation. Only a limited number of approved medications for treatment of T2D exist, and most approaches focus on similar targeting systems, such as insulins, metformin, and glucagon like 1 peptide (GLP) analogues (e.g. semaglutide and liraglutide). However, current interventions are not always successful, can have significant side effects, and do not target the root cause of T2D, insulin resistance. Accordingly, there is a substantial need for novel, more effective treatments for T2D.

Obesity is a complex disease and a major risk factor not only for T2D (with 80% of T2D patients being overweight or obese), but also for other chronic illnesses and associated mortality, such as cardiovascular diseases (e.g., heart disease, stroke), hypertension, metabolic syndrome, atherosclerosis, non-alcoholic fatty liver disease (NAFLD), various forms of dementia, including Alzheimer's disease, cancer and other diseases (Lin et al. (2021) FRONT. ENDOCRINOL. 12: 706978). It is believed that the “Western diet” is fuelling an obesity epidemic, with rates of excess body fat accumulation and obesity growing fast in adults and children across all ages. In 2020, it was estimated that 38% of the world population is classified as overweight or obese. This is projected to rise to 51% by 2035, continuing a trajectory that saw worldwide obesity nearly triple between 1975-2016 (Lobstein et al., World Obesity Atlas 2023, World Obesity Federation). First-line treatment plans for obesity start with lifestyle intervention, including diet and physical activity, which are then supplemented with medications such as GLP-1 analogues, lipase inhibitors, and appetite suppressors. However, many of these medications are of limited efficacy and/or may cause significant adverse side effects, and so there is a significant unmet need for additional obesity treatments.

The insulin receptor (IR) is a transmembrane receptor tyrosine kinase (RTK) which binds extracellular ligands (e.g., insulin) and thereby initiates the phosphoinositide 3-kinase (PI3K)/Protein Kinase B (Akt) signaling cascade in the IR signaling pathway, ultimately resulting in glucose uptake. Defects in IR signaling (e.g., decreased IR kinase activity, decreased cellular levels of IR, and/or decreased downstream pathway signaling from the IR) can contribute to a decrease in responsiveness to normal insulin levels, for example, insulin resistance, which is known to be a key factor in the development of T2D.

Protein-tyrosine phosphatase 1B (PTP1B) is an endoplasmic reticulum (ER)-associated phosphatase. PTP1B is known to interact with, and dephosphorylate, the activated insulin receptor, thereby resulting in the downregulation of the insulin receptor signaling pathway (Bock et al. (2006) DIABETES 55(12): 3536-3549). In mice, genetic ablation of the gene encoding PTP1B is known to result in enhanced insulin sensitivity and increased insulin receptor phosphorylation (Tsou et al. (2013) FRONT. NEUROSCI. 6: 192). Additionally, PTP1B −/− mice are resistant to weight gain, and remain insulin-sensitive when placed on a high-fat diet (Elchebly et al. (1999) SCIENCE 283(5407): 1544-48). PTP1B is also a negative regulator of leptin receptor. Accordingly, PTP1B has been contemplated as a therapeutic target, particularly for the treatment of obesity and type 2 diabetes (Zabolotny et al. (2002) DEV. CELL 2(4): 489-95). However, to date, no inhibitors of PTP1B have been approved in the United States or Europe for the treatment of T2D.

Accordingly, there is a need in the art for compositions and methods for treating diseases or disorders associated with insulin resistance (e.g., T2D, obesity, etc.), for example, by inhibiting certain activities of PTP1B in a subject in need thereof.

### SUMMARY OF THE INVENTION

The invention is based, in part, upon the discovery that Cellular Protein-tyrosine phosphatase 1B (PTP1B) interacts with the SH3-2 domain of the non-catalytic region of tyrosine kinase 1 protein (NCK1) to produce a cellular signaling complex involved in the insulin receptor (IR) signaling pathway. NCK1 is a cytoplasmic adaptor protein known to be involved in transducing signals from receptor tyrosine kinases to downstream signaling recipients. NCK1 modulates the expression of PTP1B, and it has been observed that NCK1 depletion enhances insulin sensitivity similar to that seen with PTP1B inhibition (Li et al. (2014) CELL COMMUN. SIGNAL. 12(1): 71).

As discussed herein, it has been found that the interaction between the SH3-2 domain of NCK1 and PTP1B can modulate the downstream signaling activity of the insulin receptor and the IR mediated pathway. By contacting NCK1 with an agent (e.g., a peptide) that interferes with the interaction between the NCK1 SH3-2 domain and PTP1B, PTP1B-mediated downregulation of the IR signaling pathway can be suppressed, thereby restoring IR signaling (see, FIGS. 1A-C). Additionally, it is contemplated that interfering with the specific complex formation between NCK1 and PTP1B (as opposed to inhibiting PTP1B activity broadly) will not suppress other activities of PTP1B, which are important for normal cellular function. Likewise, by only targeting one domain of NCK1, it is anticipated that other important functions of the NCK1 adaptor protein will not be materially impacted. Disruption of the NCK1-PTP1B complex presents a novel approach to functionally achieving selective inhibition of PTP1B.

Accordingly, the disclosure provides, among other things, agents, e.g., synthetic peptides, that bind NCK1, and particularly the SH3-2 domain of NCK1. Furthermore, the NCK1-binding synthetic peptides of the disclosure can be administered to a subject in need thereof to treat or prevent a disease or disorder, e.g., diabetes or obesity.

Accordingly, in a first aspect, the disclosure provides a synthetic peptide consisting essentially of an amino acid sequence of (i) from six to thirty or (ii) from six to fifty consecutive amino acids including an N-terminal amino acid and a C-terminal amino acid, wherein the amino acid sequence comprises the amino acid sequence of PX₁PPX₂R (SEQ ID NO: 1), wherein X₁ and X₂ are each independently any amino acid, and (a) the proline at position 1 of SEQ ID NO: 1 is the N-terminal amino acid and is optionally modified by covalent linkage to a carbonyl-containing moiety; or (b) the N-terminal amino acid is acetylated and the C-terminal amino acid is amidated.

In a second aspect, the disclosure provides a synthetic peptide consisting essentially of an amino acid sequence of six to thirty or six to fifty consecutive amino acids including an N-terminal amino acid and a C-terminal amino acid, wherein the amino acid sequence comprises the amino acid sequence of PX₁PPX₂R (SEQ ID NO: 1), wherein X₁ and X₂ are each independently any amino acid, and (a) the proline at position 1 of SEQ ID NO: 1 is the N-terminal amino acid and is optionally modified by covalent linkage to a carbonyl-containing moiety; (b) the N-terminal amino acid is chemically modified (e.g., acetylated) and/or the C-terminal amino acid is chemically modfied (e.g., amidated), or (c) the C-terminal amino acid is arginine provided that, (i) if X₂ is arginine, then X₁ is not arginine or glutamine, (ii) if X₂ is serine, then X₁ is not arginine, and (iii) if X₂ is methionine, then X₁ is not a histidine.

For each of the first and second aspects, in certain embodiments, X₁ is a charged amino acid, for example, a positively charged amino acid (e.g., arginine, histidine, and lysine) or a negatively charged amino acid (e.g., glutamic acid). In certain embodiments, X₁ is an uncharged amino acid, e.g., a hydrophobic amino acid. In certain embodiments X₁ is an amino acid selected from the group consisting of alanine, asparagine, and serine. In certain embodiments of any of the foregoing aspects and embodiments, X₂ is a charged amino acid, for example, a positively charged amino acid (e.g., arginine, histidine, or lysine) or a negatively charged amino acid (e.g., glutamic acid). In certain embodiments, X₂ is an uncharged amino acid, for example, an amino acid selected from the group consisting of alanine, asparagine, cysteine, serine, and tryptophan. In certain embodiments, X₂ is a hydrophobic amino acid, for example, an amino acid selected from the group consisting of alanine, cysteine, and tryptophan.

In each of the foregoing aspects, X₁ is selected from (i) the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine, (ii) the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine or (iii) the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. Alternatively or in addition, X₂ is selected from (i) the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, (ii) the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, or (iii) the group consisting of alanine, arginine, asparagine, cysteine, glutamic acid, glutamine, histidine, isoleucine, leucine, lysine, methionine, proline, serine, threonine, tryptophan, tyrosine, and valine.

In a third aspect, the disclosure provides a synthetic peptide consisting essentially of an amino acid sequence of seven to ten consecutive amino acids including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; (2) X₂ is a non-aromatic amino acid; (3) if X₁ is alanine and X₂ is valine, then X₃ is not proline; (4) if X₂ is proline and X₃ is arginine, then X₁ is not arginine or glutamine; and (5) if X₂ is proline and X₃ is methionine, then X₁ is not histidine. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of seven to ten consecutive amino acids including an N-terminal amino acid and a C-terminal amino acid, wherein the amino acid sequence comprises the amino acid sequence of PX₁X₂PX₃R (SEQ ID NO: 2), wherein X₁ and X₃ are each independently any amino acid and X₂ is selected from the group consisting of alanine, arginine, isoleucine, leucine, lysine, phenylalanine, proline, threonine, tyrosine, and valine; provided that if (a) X₁ is alanine and X₂ is valine, then X₃ is not proline; (b) X₂ is proline and X₃ is arginine, then X₁ is not arginine or glutamine; and (c) X₂ is proline and X₃ is methionine, then X₁ is not histidine. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a fourth aspect, the disclosure provides a synthetic peptide consisting essentially of an amino acid sequence of eleven consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid, (2); X₂ is a non-aromatic amino acid; (3) X₄ is either absent or any amino acid; and (4) if X₂ is proline and X₄ is not absent, then (i) X₁ is not threonine, (ii) X₃ is not lysine, and/or (iii) X₄ is not serine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of eleven consecutive amino acids including an N-terminal amino acid and a C-terminal amino acid, wherein the amino acid sequence comprises the amino acid sequence of PX₁X₂PX₃RX₄ (SEQ ID NO: 3), wherein X₁ and X₃ are each independently any amino acid, X₂ is selected from the group consisting of alanine, arginine, isoleucine, leucine, lysine, phenylalanine, proline, threonine, tyrosine, and valine; and X₄ is either absent or any amino acid; provided that if X₂ is proline and X₄ is not absent, then (i) X₁ is not threonine; (ii) X₃ is not lysine; and/or (iii) X₄ is not serine. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a fifth aspect, the disclosure provides a synthetic peptide consisting essentially of an amino acid sequence of twelve consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid; (2) X₂ is a non-aromatic amino acid; (3) X₄ is either absent or any amino acid; and (4) if X₂ is proline and X₄ is not absent, then (i) X₁ is not arginine, (ii) X₃ is not threonine, and/or (iii) X₄ is not threonine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of twelve consecutive amino acids including an N-terminal amino acid and a C-terminal amino acid, wherein the amino acid sequence comprises the amino acid sequence of PX₁X₂PX₃RX₄ (SEQ ID NO: 3), wherein X₁ and X₃ are each independently any amino acid; X₂ is selected from the group consisting of alanine, arginine, isoleucine, leucine, lysine, phenylalanine, proline, threonine, tyrosine, and valine; and X₄ is either absent or any amino acid; provided that if X₂ is proline and X₄ is not absent, then (i) X₁ is not arginine; (ii) X₃ is not threonine; and/or (iii) X₄ is not threonine. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a sixth aspect, the disclosure provides a synthetic peptide consisting essentially of an amino acid sequence of thirteen consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid; (2) X₂ is a non-aromatic amino acid; and (3) X₄ is either absent or any amino acid. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of thirteen consecutive amino acids including an N-terminal amino acid and a C-terminal amino acid, wherein the amino acid sequence comprises the amino acid sequence of PX₁X₂PX₃RX₄ (SEQ ID NO: 3), wherein X₁ and X₃ are each independently any amino acid; X₂ is selected from the group consisting of alanine, arginine, isoleucine, leucine, lysine, phenylalanine, proline, threonine, tyrosine, and valine; and X₄ is either absent or any amino acid. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a seventh aspect, the disclosure provides a synthetic peptide consisting essentially of an amino acid sequence of fourteen consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid; (2) X₂ is a non-aromatic amino acid; (3) X₄ is either absent or any amino acid; (4) if X₁ is alanine and X₂ is valine, then X₃ is not proline; and (5) if X₂ is proline and X₄ is absent, then (i) X₁ is not arginine, and/or (ii) X₃ is not serine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of fourteen consecutive amino acids including an N-terminal amino acid and a C-terminal amino acid, wherein the amino acid sequence comprises the amino acid sequence of PX₁X₂PX₃RX₄ (SEQ ID NO: 3), wherein X₁ and X₃ are each independently any amino acid; X₂ is selected from the group consisting of alanine, arginine, isoleucine, leucine, lysine, phenylalanine, proline, threonine, tyrosine, and valine; X₄ is either absent or any amino acid, provided that, if X₁ is alanine and X₂ is valine, then X₃ is not proline; and if X₂ is proline and X₄ is absent, then: (i) X₁ is not arginine; and/or (ii) X₃ is not serine. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In an eighth aspect, the disclosure provides a synthetic peptide consisting essentially of an amino acid sequence of fifteen consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid; (2) X₂ is a non-aromatic amino acid; (3) X₄ is either absent or any amino acid; and (4) if X₂ is proline and X₄ is not absent, then (i) if X₁ is arginine, X₃ is lysine, and X₄ is isoleucine, then X₄ is not the C-terminal amino acid; (ii) if X₄ is alanine, then X₁ is not proline and/or X₃ is not arginine; and (iii) if X₄ is proline, then (a) if X₁ is methionine, then X₃ is not asparagine; and (b) if X₁ is arginine, then X₃ is not glycine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a nineth aspect, the disclosure provides a synthetic peptide consisting essentially of an amino acid sequence of fifteen consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid; (2) X₂ is a non-aromatic amino acid; (3) X₄ is either absent or any amino acid; and (4) if X₂ is proline and X₄ is not absent, then: (i) if X₁ is arginine, X₃ is lysine, and X₄ is isoleucine, then X₄ is not the C-terminal amino acid and/or the N-terminal amino acid is not a proline; (ii) if X₄ is alanine, then X₁ is not proline and/or X₃ is not arginine; and (iii) if X₄ is proline, then: (a) if X₁ is methionine, then X₃ is not asparagine; and (b) if X₁ is arginine, then X₃ is not glycine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of fifteen consecutive amino acids including an N-terminal amino acid and a C-terminal amino acid, wherein the amino acid sequence comprises the amino acid sequence of PX₁X₂PX₃RX₄ (SEQ ID NO: 3), wherein X₁ and X₃ are each independently any amino acid; X₂ is selected from the group consisting of alanine, arginine, isoleucine, leucine, lysine, phenylalanine, proline, threonine, tyrosine, and valine; X₄ is either absent or any amino acid; provided that if X₂ is proline and X₄ is not absent, then: (i) if X₁ is arginine, X₃ is lysine, and X₄ is isoleucine, then X₄ is not the C-terminal amino acid and/or the N-terminal amino acid is not a proline; (ii) if X₄ is alanine, then X₁ is not proline and/or X₃ is not arginine; and (iii) if X₄ is proline, then: (a) if X₁ is methionine, then X₃ is not asparagine; and (b) if X₁ is arginine, then X₃ is not glycine. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a tenth aspect, the disclosure provides a synthetic peptide consisting essentially of an amino acid sequence of (i) sixteen to thirty (including intervening integers) or (ii) sixteen to fifty (including intervening integers), consecutive amino acids including an N-terminal amino acid and a C-terminal amino acid, wherein the amino acid sequence comprises the amino acid sequence of PX₁X₂PX₃RX₄ (SEQ ID NO: 3), wherein: (1) X₁ and X₃ are each independently any amino acid; (2) X₂ is a non-aromatic amino acid; (3) X₄ is either absent or any amino acid; and (4) wherein, if the synthetic peptide consists essentially of twenty consecutive amino acids, then the four most C-terminal amino acid residues of the synthetic peptide is not SLSR (SEQ ID NO: 85). In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In a related aspect, the disclosure provides a synthetic peptide consisting essentially of an amino acid sequence of (i) sixteen to thirty or (ii) sixteen to 50, consecutive amino acids including an N-terminal amino acid and a C-terminal amino acid, wherein the amino acid sequence comprises the amino acid sequence of PX₁X₂PX₃RX₄ (SEQ ID NO: 3), wherein X₁ and X₃ are each independently any amino acid; X₂ is selected from the group consisting of alanine, arginine, isoleucine, leucine, lysine, phenylalanine, proline, threonine, tyrosine, and valine; and X₄ is either absent or any amino acid; and wherein, if the synthetic peptide consists essentially of twenty consecutive amino acids, then the four most C-terminal amino acid residues of the synthetic peptide is not SLSR (SEQ ID NO: 85). In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. In certain embodiments, if the proline at position 1 of SEQ ID NO: 3 is the N-terminal amino acid of the synthetic peptide, then the N-terminal amino acid is modified with a chemical moiety (e.g., by covalent linkage to a carbonyl-containing moiety).

In certain embodiments of the fifth to tenth aspects, X₁ is a charged amino acid, for example, a positively charged amino acid (e.g., arginine, histidine, or lysine) or a negatively charged amino acid (e.g., glutamic acid). In certain embodiments, X₁ is an uncharged amino acid, for example, a hydrophobic amino acid. In certain embodiments, X₁ is selected from the group consisting of alanine, asparagine, and serine. Alternatively or in addition, in certain embodiments, X₂ is an uncharged, non-aromatic amino acid, for example, alanine or proline. In certain embodiments, X₂ is a hydrophobic, non-aromatic amino acid, e.g., alanine. In certain embodiments, X₂ is a charged amino acid, e.g., a positively charged amino acid, e.g., arginine. Alternatively or in addition, in certain embodiments, X₃ is a charged amino acid, for example, a positively charged amino acid (e.g., arginine, histidine, or lysine) or a negatively charged amino acid (e.g., glutamic acid). In certain embodiments, X₃ is an uncharged amino acid, for example, an amino acid selected from the group consisting of alanine, asparagine, cysteine, serine, and tryptophan. In certain embodiments, X₃ is a hydrophobic amino acid, for example, an amino acid selected from the group consisting of alanine, cysteine, and tryptophan.

In certain embodiments of the sixth to tenth aspects, X₄ is a charged amino acid, e.g., a positively charged amino acid (e.g., arginine, histidine, and lysine). In certain embodiments, X₄ is a negatively charged amino acid (e.g., aspartic acid or glutamic acid). In certain embodiments, X₄ is an uncharged amino acid (e.g., alanine, asparagine, cysteine, glycine, isoleucine, leucine, methionine, phenylalanine, proline, serine, threonine, tryptophan, tyrosine, or valine. In certain embodiments, X₄ is an aliphatic amino acid (e.g., alanine, glycine, isoleucine, leucine, proline, or valine). In certain embodiments, X₄ is an aromatic amino acid (e.g., phenylalanine, tryptophan, or tyrosine). In certain embodiments, X₄ is a sulfur-containing amino acid (e.g., cysteine or methionine. In certain embodiments, X₄ is an amino acid with a hydroxyl-containing side chain (e.g., serine or threonine). In certain embodiments, X₄ is an acidic amino acid (e.g., aspartic acid or glutamic acid). In certain embodiments, X₄ is a basic amino acid (e.g., arginine, histidine, or lysine). In certain embodiments, X₄ is an amino acid with an amide-containing sidechain (e.g., asparagine or glutamine).

In certain embodiments of the fifth to tenth aspects, X₁ is selected from the group consisting of (i) alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine, (ii) alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine, or (iii) alanine, arginine, asparagine, aspartic acid, cysteine, glutamic acid, glutamine, histidine, isoleucine, leucine, lysine, methionine, phenylalanine, proline, serine, threonine, tyrosine, and valine. Alternatively, or in addition, X₂ is selected from the group consisting of (i) alanine, arginine, isoleucine, leucine, lysine, proline, threonine, and valine or (ii) alanine, isoleucine, proline, and valine, or isoleucine, proline, and valine. Alternatively or in addition, X₃ is selected from the group consisting of (i) alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, (ii) alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, or (iii) alanine, arginine, asparagine, cysteine, glutamic acid, glutamine, histidine, isoleucine, leucine, lysine, methionine, proline, serine, threonine, tryptophan, tyrosine, and valine.

In an eleventh aspect, the disclosure provides a synthetic peptide consisting essentially of an amino acid sequence of from (i) six to thirty or (ii) six to fifty, consecutive amino acids including an N-terminal amino acid and a C-terminal amino acid, wherein the amino acid sequence comprises the amino acid sequence of PRPPKRX₁ (SEQ ID NO: 4), wherein: (1) X₁ is absent or any amino acid, and (2) if X₁ is isoleucine and the synthetic peptide is a 15-mer, then X₁ is not the C-terminal amino acid, and optionally proline is not the N-terminal amino acid. In certain embodiments, if the proline at position 1 of SEQ ID NO: 4 is the N-terminal amino acid of the synthetic peptide, then the N-terminal amino acid is modified with a chemical moiety (e.g., by covalent linkage to a carbonyl-containing moiety).

In certain embodiments of the eleventh aspect, X₁ is a charged amino acid, such as a positively charged amino acid (e.g., arginine, histidine, or lysine) or a negatively charged amino acid (e.g., aspartic acid or glutamic acid). In certain embodiments, X₁ is an uncharged amino acid (e.g., alanine, asparagine, cysteine, glycine, isoleucine, leucine, methionine, phenylalanine, proline, serine, threonine, tryptophan, tyrosine, or valine). In certain embodiments, X₁ is an aliphatic amino acid (e.g., alanine, glycine, isoleucine, leucine, proline, or valine). In certain embodiments, the synthetic peptide comprises the amino acid sequence of PRPPKRI (SEQ ID NO: 5), PRPPKRIL (SEQ ID NO: 6), or PRPPKRILE (SEQ ID NO: 7). In certain embodiments, X₁ is an aromatic amino acid (e.g., phenylalanine, tryptophan, or tyrosine). In certain embodiments, X₁ is a sulfur-containing amino acid (e.g., cysteine or methionine). In certain embodiments, X₁ is an amino acid with a hydroxyl-containing side chain (e.g., serine or threonine. In certain embodiments, X₁ is an acidic amino acid (e.g., aspartic acid or glutamic acid). In certain embodiments, X₁ is a basic amino acid (e.g., arginine, histidine, or lysine). In certain embodiments, X₁ is an amino acid with an amide-containing sidechain (e.g., asparagine or glutamine). In certain embodiments, the synthetic peptide has the amino acid sequence of

(SEQ ID NO: 8)
PRPPKR,
(SEQ ID NO: 9)
PPRPPKR
or
(SEQ ID NO: 10)
PPPEHIPPPPRPPKR.

In certain embodiments of aspects 1 through 11, the foregoing synthetic peptides exhibit binding to the SH3-2 domain of NCK1 with a K_{D} of ≤1,000 μM, for example, as determined by isothermal titration calorimetry (ITC). In certain embodiments, the foregoing synthetic peptides exhibit binding to the SH3-2 domain of NCK1 with a K_{D} of ≤600 μM, for example, as determined by ITC. In certain embodiments, the foregoing synthetic peptides exhibit binding to the SH3-2 domain of NCK1 with a K_{D} of ≤300 μM, for example, as determined by ITC.

In a twelfth aspect, the disclosure provides a synthetic peptide consisting essentially of an amino acid sequence of (i) six to thirty or (ii) six to fifty, consecutive amino acids including an N-terminal amino acid and a C-terminal amino acid, wherein the amino acid sequence comprises the amino acid sequence of PX₁X₂PX₃R (SEQ ID NO: 2), wherein X₁ and X₃ are each independently any amino acid; X₂ is selected from the group consisting of selected from the group consisting of alanine, arginine, isoleucine, leucine, lysine, phenylalanine, proline, threonine, tyrosine, and valine; and the synthetic peptide exhibits binding to the SH3-2 domain of NCK1 with a K_{D} of <1,000 μM, for example, as determined by ITC. In certain embodiments, the synthetic peptide exhibits binding to the SH3-2 domain of NCK1 with a K_{D} of 600 μM or less <300 μM, for example, as determined by ITC. In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine. It is contemplated that the synthetic peptide consists essentially of an amino acid sequence of at least seven, at least eight, at least nine, at least ten, at least eleven, at least twelve, at least thirteen, at least fourteen, or at least fifteen consecutive amino acids. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In certain embodiments of any of the foregoing peptides, at least one, two or more amino acids are covalently linked to a chemical moiety not present in a natural amino acid. For example, it is contemplated that the N-terminal amino acid can be chemically modified, for example, the N-terminal amino acid can comprise a chemical modification not present in a naturally occuring amino acid. For example, in certain embodiments, the N-terminal amino acid is acetylated. In certain embodiments, the N-terminal amino acid is H-pyroglutamic acid (H-Pyr). Alternatively or in addition, it is contemplated that the C-terminal amino acid can be chemically modified, for example, the C-terminal amino acid can comprise a chemical modification not present in a naturally occuring amino acid. For example, in certain embodiments, the C-terminal amino acid is amidated. In certain embodiments, the C-terminal amino acid comprises an ethylamide (NHEt) group.

In certain embodiments, the synthetic peptide optionally comprises an amino acid residue (X₀) that is N-terminal to position 1 in SEQ ID NO: 1, 2, 3, or 4. The amino acid residue at position X₀ can be selected from (i) the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, or (ii) the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

It is contemplated that the synthetic peptides disclosed herein can be provided in a composition containing a salt, for example, an acetate salt (e.g., bis-acetate or tri-acetate).

In a related aspect, the disclosure provides a pharmaceutical composition comprising any one of the foregoing synthetic peptides or a salt thereof (e.g., an acetate salt), and a pharmaceutically acceptable carrier.

In a related aspect, the disclosure provides a pharmaceutical composition comprising a synthetic peptide consisting essentially of an amino acid sequence of (i) six to thirty or (ii) six to fifty, consecutive amino acids including an N-terminal amino acid and a C-terminal amino acid, wherein the amino acid sequence comprises the amino acid sequence of PX₁X₂PX₃R (SEQ ID NO: 2), wherein X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine; X₂ is selected from the group consisting of alanine, arginine, isoleucine, leucine, lysine, phenylalanine, proline, threonine, tyrosine, and valine; and X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In certain embodiments, X₁ is selected from (i) the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine or (ii) alanine, arginine, asparagine, aspartic acid, cysteine, glutamic acid, glutamine, histidine, isoleucine, leucine, lysine, methionine, phenylalanine, proline, serine, threonine, tyrosine, and valine. Alternatively or in addition to each of the foregoing, X₂ is selected from (i) the group consisting of alanine, arginine, isoleucine, leucine, lysine, proline, threonine, tyrosine, and valine, (ii) the group consisting of alanine, isoleucine, proline, and valine or (iii) the group consisting of isoleucine, proline, and valine. Alternatively or in addition to each of the foregoing, X₃ is selected from (i) the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine or (ii) the group consisting of alanine, arginine, asparagine, cysteine, glutamic acid, glutamine, histidine, isoleucine, leucine, lysine, methionine, proline, serine, threonine, tryptophan, tyrosine, and valine.

The pharmaceutical composition is sterile, is can be provided as a dried composition (e.g., freeze dried or lyophilized) or as an aqueous composition (e.g., the peptide solubilized in water for injection or suitable buffer) such as an aqueous composition suitable for injection. The concentration of the synthetic peptide in the aqueous composition can be between about 150 mg/mL and about 400 mg/mL, or between about 200 mg/mL and about 300 mg/mL. Depending upon the mode of delivery, the disclosure provides a delivery system comprising (i) delivery device (e.g., a syringe such as a prefilled syringe) and (ii) a composition comprising any one or more of the peptides disclosed herein or a pharmaceutical composition disclosed herein.

In another aspect, the disclosure provides a method of modulating the activity of NCK1, the method comprising contacting NCK1 with an agent that disrupts an interaction between PTP1B and NCK1 thereby to modulate the activity of NCK1. Exemplary agents include, for example, a protein (e.g., antibody or antigen binding fragment thereof) or peptide (e.g., a peptide disclosed herein), that disrupts the interaction between PTP1B and NCK1 via the SH3-2 domain of NCK1. The contacting step disrupts the interaction between PTP1B and NCK1. In certain embodiments, the step of contacting results in modulating the activity of PTP1B.

In another aspect, the disclosure provides a method of modulating the activity of NCK1, the method comprising contacting NCK1 with any one of the foregoing synthetic peptides or pharmaceutical compositions, thereby to modulate the activity of NCK1. In certain embodiments, the step of contacting disrupts the interaction between PTP1B and NCK1. In certain embodiments, the step of contacting results in modulating the activity of PTP1B.

In another aspect, the disclosure provides a method of decreasing body fat mass in a subject in need thereof, the method comprising administering to the subject an effective amount of a synthetic peptide described herein or a pharmaceutical composition comprising a synthetic peptide disclosed herein and a pharmaceutically acceptable carrier, thereby to decrease body fat mass of the subject, wherein the synthetic peptide consists essentially of an amino acid sequence of (i) six to thirty or (ii) or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is a non-aromatic amino acid. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a related aspect, the disclosure provides a method of decreasing body fat mass in a subject in need thereof, the method comprising administering to the subject an effective amount of a synthetic peptide described herein or a pharmaceutical composition comprising a synthetic peptide disclosed herein and a pharmaceutically acceptable carrier, thereby to decrease body fat mass of the subject, wherein the synthetic peptide consists essentially of an amino acid sequence of (i) six to thirty or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is selected from the group consisting of alanine, arginine, isoleucine, leucine, lysine, phenylalanine, proline, threonine, tyrosine, and valine. In certain embodiments, if the proline at position 1 of SEQ ID NO: 2 is the N-terminal amino acid of the synthetic peptide, then the N-terminal amino acid is modified with a chemical moiety (e.g., covalent linkage to a carbonyl-containing moiety).

In a related aspect, the disclosure provides a method of decreasing body fat mass in a subject in need thereof, the method comprising administering to the subject an effective amount of any one of the foregoing synthetic peptides or pharmaceutical compositions, thereby to decrease body fat mass of the subject.

In another aspect, the disclosure provides a method of treating obesity in a subject in need thereof, the method comprising administering to the subject an effective amount of a synthetic peptide or a pharmaceutical composition comprising the synthetic peptide and a pharmaceutically acceptable carrier, thereby to treat obesity in the subject, wherein the synthetic peptide consists essentially of an amino acid sequence of (i) six to thirty or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is a non-aromatic amino acid. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a related aspect, the disclosure provides a method of treating obesity in a subject in need thereof, the method comprising administering to the subject an effective amount of a synthetic peptide or a pharmaceutical composition comprising the synthetic peptide and a pharmaceutically acceptable carrier, thereby to treat obesity in the subject, wherein the synthetic peptide consists essentially of an amino acid sequence of (i) six to thirty or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is selected from the group consisting of alanine, arginine, isoleucine, leucine, lysine, phenylalanine, proline, threonine, tyrosine, and valine. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a related aspect, the disclosure provides a method of treating obesity in a subject in need thereof, the method comprising administering to the subject an effective amount of any one of the foregoing synthetic peptides or pharmaceutical compositions, thereby to treat obesity in the subject.

In another aspect, the disclosure provides a method of treating or preventing diabetes (e.g., Type 2 diabetes) in a subject in need thereof, the method comprising administering to the subject an effective amount of a synthetic peptide described herein or a pharmaceutical composition comprising the synthetic peptide and a pharmaceutically acceptable carrier, thereby to treat or prevent diabetes in the subject, wherein the synthetic peptide consists essentially of an amino acid sequence of (i) six to thirty or (ii) six to fifty consecutive amino acids including an N-terminal amino acid and a C-terminal amino acid, wherein the amino acid sequence comprises the amino acid sequence of PX₁X₂PX₃R (SEQ ID NO: 2), wherein (1) X₁ and X₃ are each independently any amino acid; and (2) X₂ is a non-aromatic amino acid. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a related aspect, the disclosure provides a method of treating or preventing diabetes (e.g., Type 2 diabetes) in a subject in need thereof, the method comprising administering to the subject an effective amount of a synthetic peptide or a pharmaceutical composition comprising the synthetic peptide and a pharmaceutically acceptable carrier, thereby to treat or prevent diabetes in the subject, wherein the synthetic peptide consists essentially of an amino acid sequence of (i) six to thirty or (ii) six to fifty consecutive amino acids including an N-terminal amino acid and a C-terminal amino acid, wherein the amino acid sequence comprises the amino acid sequence of PX₁X₂PX₃R (SEQ ID NO: 2), wherein (1) X₁ and X₃ are each independently any amino acid; and (2) X₂ is selected from the group consisting of alanine, arginine, isoleucine, leucine, lysine, phenylalanine, proline, threonine, tyrosine, and valine. In certain embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In a related aspect, the disclosure provides a method of treating or preventing diabetes (e.g., Type 2 diabetes) in a subject in need thereof, the method comprising administering to the subject any one of the foregoing synthetic peptides or pharmaceutical compositions, thereby to treat or prevent diabetes in the subject.

In certain embodiments of the foregoing methods, the subject is a mammal, e.g., a human. In each of the foregoing methods, the administration comprises systemic administration (e.g., via subcutaneous administration).

In each of the foregoing methods, in certain embodiments, X₁ is a charged amino acid, for example, a positively charged amino acid (e.g., arginine, histidine, or lysine) or a negatively charged amino acid (e.g., glutamic acid). In certain embodiments, X₁ is an uncharged amino acid, for example, a hydrophobic amino acid. In certain embodiments, X₁ is selected from the group consisting of alanine, asparagine, and serine. Alternatively or in addition, in certain embodiments of the foregoing methods, X₂ is an uncharged, non-aromatic amino acid. In certain embodiments, X₂ is alanine or proline. In certain embodiments, X₂ is a hydrophobic, non-aromatic amino acid, e.g., alanine. In certain embodiments, X₂ is a charged amino acid, e.g., a positively charged amino acid, e.g. arginine. Alternatively or in addition to each of the foregoing methods, X₃ is a charged amino acid, for example, a positively charged amino acid (e.g., arginine, histidine, or lysine) or a negatively charged amino acid (e.g., glutamic acid). In certain embodiments, X₃ is an uncharged amino acid, for example, an amino acid selected from the group consisting of alanine, asparagine, cysteine, serine, and tryptophan. In certain embodiments, X₃ is a hydrophobic amino acid, for example, an amino acid selected from the group consisting of alanine, cysteine, and tryptophan.

These and other aspects and features of the invention are described in the following detailed description and claims.

### BRIEF DESCRIPTION OF THE DRAWINGS

The invention can be more completely understood with reference to the following drawings, in which:

FIG. 1A is a schematic that depicts interaction between PTP1B (labeled “2”) and NCK1 (labeled “1”) (upper panel) and disruption between the two proteins using a NCK1-binding modulating agent (labeled “3”), such as a synthetic peptide disclosed herein (lower panel). FIGS. 1B-1C are schematics depicting the impact of NCK1 and PTP1B on insulin receptor signaling (FIG. 1B), and the how the use of a NCK1-binding modulating agent can disrupt the interaction between NCK1 and PTP1B and consequently enhance insulin pathway signaling, thereby restoring insulin sensitivity (FIG. 1C). The term “IR” represents insulin receptor, the term “IRS1” represents insulin receptor substrate 1, and the term “PI3K/AKT” represents “phosphoinositide 3-kinase/protein kinase B.”

FIGS. 2A-2D depict immunoblots showing co-immunoprecipitation of FLAG-PTP1B and HA-NCK1 in HEK-293 cells co-transfected with both proteins. Either FLAG-PTP1B (FIG. 2A) or HA-NCK1 (FIG. 2B) was immunoprecipitated from the co-transfected cells, and Western blotting was used to detect the presence of the other fusion protein in the cell lysate (lane 2) and the IP eluate (lane 4). Non-transfected HEK-293 cells were used as negative controls (lanes 1 and 3). Ponceau staining was used to measure total protein in the FLAG-PTP1B IP assay (FIG. 2C) and in the HA-NCK1 IP assay (FIG. 2D).

FIG. 3 depicts an immunoblot showing co-immunoprecipitation of His-PTP1B and GST-tagged full-length NCK1 (“N” or “GST NCK1”) or GST-tagged SH3-2 domain of NCK1 (“SH3-2” or “GST SH3-2”). Bead-bound GST fusion protein was co-incubated with the indicated concentration of His-PTP1B, and His-PTP1B was detected in the IP eluate by Western blotting (top image). GST alone (“X” or “Control GST”) and GST-tagged PTP1B (“P” or “GST PTP1B”) were used as negative controls. “E protein” refers to the purified SH3-2 domain of NCK1. Ponceau staining was used to measure total protein (bottom image).

FIG. 4A depicts an immunoblot showing that certain peptides prevent co-immunoprecipitation of FLAG-PTP1B and GST-tagged SH3-2 domain of NCK1 (“SH3”). GST was fused to full-length NCK1 (“Nck1”), all SH3 domains of NCK1 (“SH3”), the NCK1 SH2 domain (“SH2”), the NCK1 SH3-1 domain (“SH3 1”), the NCK1 SH3-2 domain (“SH3 2”), and the NCK1 SH3-3 domain (“SH3 3”). GST alone was used as a negative control (“GST”). FLAG-PTP1B was incubated with the indicated bead-bound GST-fusion protein, and FLAG-PTP1B was detected in the IP eluate by western blotting. The outlined region in column “SH3-2” identifies the PTP1B band. For GST-SH3-2 and FLAG-PTP1B, the mixture was further incubated with one of peptides A through K, (1 mM). FIG. 4B depicts an input control for the immunoblot. FIG. 4C depicts a Ponceau stain for the immunoblot, which was used to measure total protein. FIG. 4D depicts a legend summarizing the peptides tested in columns A through K, and summarizes which peptides did and did not break the interaction between FLAG-PTP1B and SH3-2, based on the results in FIG. 4A.

FIG. 5 is a bar graph showing the efflux ratios of different compounds in a Caco-2 assay. The efflux ratio of MD20 (0.252) is indicated as a bolded line.

FIG. 6 is a graph showing MD20 concentrations in the indicated tissues in a mouse following intraperitoneal (IP) administration of the peptide at a dose of 30 mg/kg. Tissue samples were collected at 0, 0.25, 0.5, 1, 2, 4, 8, and 24 hours post-injection. MD20 was not detected in liver tissue at any time point.

FIGS. 7A-7B are graphs showing body weight changes in mice following daily IP injection of MD17 or MD20 at a dose of 15 mg/kg or 30 mg/kg for 28 consecutive days. Mice injected with a PBS solution were used as a control. Results are reported as mean body weight of the treatment group at weekly timepoints (FIG. 7A) and as the percentage change in body weight at the conclusion of the 4-week protocol (FIG. 7B).

FIGS. 8A-8D are bar graphs showing changes in lean mass and fat mass (as measured by Echo MRI scan) in mice following daily IP injection of MID7 or MD20 at a dose of 15 mg/kg or 30 mg/kg for 28 consecutive days. Mice injected with a PBS solution were used as a control. Results are reported as mean lean mass (FIG. 8A) and mean fat mass (FIG. 8B) pre- and post-treatment, and as the percentage change in lean mass (FIG. 8C) and fat mass (FIG. 8D) at the conclusion of the 4-week protocol. Data represent mean±SEM; statistical differences were determined using one-way ANOVA with Bonferroni post hoc tests.

FIGS. 9A-9F are bar graphs showing metabolic markers and liver toxicity markers in mice following daily IP injection of MD17 or MD20 at a dose of 15 mg/kg or 30 mg/kg for 28 consecutive days. Mice injected with a PBS solution were used as a control. Mice were fasted for 5 hours at the conclusion of the experiment, and blood and serum samples were analyzed for blood glucose (FIG. 9A), insulin (FIG. 9B), leptin (FIG. 9C), free fatty acid (FAA, FIG. 9D), and for liver toxicity markers alanine aminotransferase (ALT) (FIG. 9E) and aspartate aminotransferase (AST) (FIG. 9F). Data represent mean±SEM; statistical differences were determined using one-way ANOVA with Bonferroni post hoc tests.

FIG. 10 depicts a diagram summarizing the mouse high-fat diet (HFD) study described in Example 7, which was used to investigate the effect of daily IP MD20 administration (30 mg/kg) on a mouse model of obesity and type 2 diabetes.

FIGS. 11A-11B are graphs showing body weight changes in HFD-fed mice following daily administration of MD20. After eight weeks of HFD feeding, mice were administered a daily IP injection of MD20 (30 mg/kg) or a vehicle control for 7 weeks. Results are reported as mean body weight of the treatment group at weekly timepoints (FIG. 11A) and as the percentage change in body weight at weekly timepoints post-treatment with MD20 (FIG. 11B).

FIGS. 12A-12F are graphs showing changes in lean mass and fat mass (as measured by Echo MRI scan) in HFD-fed mice following daily administration of MD20. After eight weeks of HFD feeding, mice were administered a daily IP injection of MD20 (30 mg/kg) or a vehicle control for 7 weeks. Results for the indicated weeks post-treatment are reported as mean fat mass (FIG. 12A, FIG. 12B), mean lean mass (FIG. 12A, FIG. 12D), and percentage change in fat mass (FIG. 12C). Results at the conclusion of the protocol are reported as percentage change in fat mass (FIG. 12E) and the percentage change in lean mass (FIG. 12F). Data represent mean±SEM; statistical differences were determined using repeated measures two-way ANOVA followed by a Bonferroni post hoc test or two-tailed student's t-test.

FIGS. 13A-13B are graphs showing the results of an insulin tolerance test (ITT) in HFD-fed mice following 7 weeks of daily IP administration of MD20 (30 mg/kg) or a vehicle control. At the conclusion of the study, mice were 5-hour fasted and subsequently injected IP with 0.6 mU/g insulin. Blood glucose levels were measured at 0, 15, 30, 60, and 90 minutes post-insulin injection. Results were reported as blood glucose concentration (FIG. 13A) and as percentage change in blood glucose levels (FIG. 13B). Data represent mean±SEM; statistical differences were determined using repeated measures two-way ANOVA followed by Bonferroni post hoc test.

FIG. 14 is a bar graph showing changes in blood glucose levels in HFD-fed mice before and during the course of 7 weeks of daily IP administration of MD20 (30 mg/kg) or a vehicle control. Blood glucose levels were measured prior to starting an HFD diet (“pre-HFD”), following administration of an HFD diet but prior to treatment (“pre-treat”), and at the indicated timepoints following daily treatment with MD20 or vehicle control (“week #”). Data represent mean±SEM; statistical differences were determined using one-way ANOVA followed by Bonferroni post hoc test.

FIG. 15 is a graph showing the results of an glucose tolerance test (GTT) in HFD-fed mice following 7 weeks of daily IP administration of MD20 (30 mg/kg) or a vehicle control. At the conclusion of the study, mice were 5-hour fasted and subsequently injected IP with 2 mg/g glucose. Blood glucose levels were measured at 0, 15, 30, 60, and 90 minutes post-glucose injection. Results were reported as blood glucose concentration. Data represent mean±SEM; statistical differences were determined using repeated measures two-way ANOVA followed by Bonferroni post hoc test.

FIGS. 16A-16F are graphs showing serum levels of various markers in HFD-fed mice following 7 weeks of daily IP administration of MD20 (30 mg/kg) or a vehicle control. Mice were fasted for 5 hours at the conclusion of the experiment, and serum samples were analyzed for levels of insulin (FIG. 16A), leptin (FIG. 16B), cholesterol (FIG. 16C), free fatty acids (FIG. 16D), triglycerides (FIG. 16E), and liver triglycerides (FIG. 16F). Data represent mean±SEM; statistical differences were determined using two tailed student's t-test.

FIGS. 17A-17B are graphs showing serum ALT and AST activity in HFD-fed mice following 7 weeks of daily IP administration of MD20 (30 mg/kg) or a vehicle control. Mice were fasted for 5 hours at the conclusion of the experiment, and serum samples were analyzed for alanine aminotransferase (ALT) activity (FIG. 17A) and aspartate aminotransferase (AST) activity (FIG. 17B). Data represent mean±SEM; statistical differences were determined using two tailed student's t-test.

FIGS. 18A-18B are graphs showing changes in body weight in db/db mice following daily subcutaneous (SC) administration of either 15, 30, or 45 mg/kg MD18 (FIG. 18A) or 15, 30, or 45 mg/kg MD20 (FIG. 18B) for the indicated number of days, as compared to a vehicle control (“PBS”). Data represent means±SEM.

FIGS. 19A-19D are graphs showing changes in changes in fat mass (FIG. 19A and FIG. 19C) and lean mass (FIG. 19B and FIG. 19D) in db/db mice following daily SC administration of MD18 (15, 30, or 45 mg/kg), MD20 (15, 30, or 45 mg/kg), or a vehicle control for 41 days. Data are presented as absolute mass (FIG. 19A and FIG. 19B) and as percent change in mass pre- versus post-treatment (FIG. 19C and FIG. 19D). Data represent means±SEM.

FIGS. 20A-20E are graphs showing the results of a glucose tolerance test (GTT) in db/db mice. A GTT was carried after 4 weeks of daily SC administration of either MD18 (15, 30, or 45 mg/kg;), MD20 (15, 30, or 45 mg/kg;), or a vehicle control, as indicated. The GTT was carried out by IP injecting 5 h-fasted mice with glucose. Blood glucose levels were measured pre-glucose-injection and at 15, 30, 60, and 90 minutes post-glucose injection, and results were reported as blood glucose concentration (FIG. 20A and FIG. 20B), as percent change in blood glucose concentration pre- versus post-treatment (FIG. 20C and FIG. 20D), and as the glucose area under the curve (AUC; FIG. 20E). Data represent means SEM.

FIGS. 21A-21C are graphs showing the results of an insulin tolerance test (ITT) in db/db mice following 6 weeks of daily SC administration of either MD18 (15, 30, or 45 mg/kg;

FIG. 21A), MD20 (15, 30, or 45 mg/kg; FIG. 21B), or a vehicle control. The ITT was carried out by injecting 5 h-fasted mice IP with 1 IU/kg insulin. Blood glucose levels were measured pre-insulin-injection and at 15, 30, 60, and 90 minutes post-insulin injection. Results were reported as percentage change in glucose levels (FIGS. 21A-21B) and as the glucose AUC (FIG. 21C). Data represent means±SEM.

FIGS. 22A-22E are bar graphs showing basal blood glucose levels in db/db mice over after 1 week (FIG. 22A), 2 weeks (FIG. 22B), 3 weeks (FIG. 22C), 4 weeks (FIG. 22D), or five weeks (FIG. 22E) of daily SC administration of either MD18 (15, 30, or 45 mg/kg), MD20 (15, 30, or 45 mg/kg), or a vehicle control (“PBS”). Blood glucose was measured using AlphaTRAK 2 glucose strips and glucometer (Abbott, UK) or by glucose assay (AB65333, Abcam), as indicated. Data represent means±SEM.

FIGS. 23A-23B are graphs showing serum levels of insulin (FIG. 23A) and leptin (FIG. 23B) in 5-hour-fasted db/db mice following about 6 weeks of daily SC administration of MD18 (15, 30, or 45 mg/kg), MD20 (15, 30, or 45 mg/kg), or a vehicle control (“PBS”). Data represent mean±SEM.

FIGS. 24A-24C are graphs showing body weight changes in LDLR−/− mice following daily administration of MD20. After eight weeks of HFD feeding, mice were administered a daily IP injection of MD20 (30 mg/kg) or a vehicle control for 6 weeks. Results are reported as mean body weight at weekly timepoints post-HFD-feeding (FIG. 24A) or post-MD20 treatment (FIG. 24C), or as percentage change in body weight post-treatment (FIG. 24B). Data represent mean±SEM; statistical differences were determined using two way ANOVA followed by Bonferroni post hoc tests.

FIGS. 25A-25F are graphs showing changes in lean mass and fat mass (as measured by Echo MRI scan) in HFD-fed LDLR−/− mice following daily administration of MD20 (30 mg/kg) or a vehicle control for 6 weeks. Data were recorded at the indicated timepoints post-treatment, and results are reported as absolute fat mass (FIG. 25A), percentage change in fat mass (FIG. 25B and FIG. 25C), absolute lean mass (FIG. 25D), and percentage change in lean mass (FIG. 25E and FIG. 25F).

Data represent mean±SEM; statistical differences were determined using repeated measures two-way ANOVA followed by a Bonferroni post hoc test or two-tailed student's t-test.

FIGS. 26A-26D are graphs showing the results of an insulin tolerance test (ITT) in HFD-fed LDLR−/− mice following 6 weeks of daily IP administration of either MD20 (30 mg/kg) or a vehicle control. At the conclusion of the study, mice were 5 h-fasted and subsequently injected IP with 0.6 mU/g insulin. Blood glucose levels were measured pre-insulin injection (basal glucose concentration; FIG. 26C) and at 15, 30, 60, and 90 minutes post-insulin injection. Results were reported as blood glucose concentration (FIG. 26A), as percentage change in glucose levels (FIG. 26B), and as the glucose AUC FIG. 26D).

Data represent mean±SEM; statistical differences were determined using (repeated measures two-way ANOVA followed by Bonferroni post hoc test.

FIGS. 27A-27C are graphs showing the results of a glucose tolerance test (GTT) in HFD-fed LDLR−/− mice. A GTT was carried after 0 weeks (FIG. 27A), 3 weeks (FIG. 27B), and 5 weeks (FIG. 27C) of daily IP administration of either MD20 (30 mg/kg) or a vehicle control. The GTT was carried out by IP injecting 5 h-fasted mice with 2 mg/g glucose. Blood glucose levels were measured at 0, 15, 30, 60, and 90 minutes post-glucose injection, and results were reported as blood glucose concentration. Data represent mean±SEM; statistical differences were determined using repeated measures two-way (FIGS. 27A-27B) or one-way (FIG. 27C) ANOVA followed by Bonferroni post hoc test.

FIGS. 28A-28F are graphs showing levels of various hormones, lipids, and hepatic toxicity markers in HFD-fed LDLR−/− mice following 6 weeks of daily IP administration of MD20 (30 mg/kg) or a vehicle control. Mice were fasted for 5 hours at the conclusion of the experiment, and serum samples were analyzed for levels of insulin (FIG. 28A), leptin (FIG. 28B), cholesterol (FIG. 28C), free fatty acids (FIG. 28D), triglycerides (FIG. 28E), alanine aminotransferase (ALT) activity (FIG. 28G), and aspartate aminotransferase (AST) activity (FIG. 28H). Liver triglyceride levels were also analyzed (FIG. 28F). Data represent mean±SEM; statistical differences were determined using two tailed student's t-test.

FIGS. 29A-29B summarize the extent of aortic plaque coverage in HFD-fed LDLR−/− mice following 6 weeks of daily IP administration of MD20 (30 mg/kg) or a vehicle control. FIG. 29A is a graph showing percentage of aortic plaque coverage in MD20-treated mice (“Treatment”) as compared to control mice (“Vehicle”). Data represent mean SEM; statistical differences were determined using one- or two-tailed student's t-test. FIG. 29B is a photograph depicting a mouse aorta stained with Sudan IV to measure plaque coverage.

FIGS. 30A-30E are graphs showing expression of various genes in tissues of HFD-fed LDLR−/− mice following 6 weeks of daily IP administration of MD20 (30 mg/kg) or a vehicle control. Mice were fasted for 5 hours at the conclusion of the experiment, liver and white adipose tissue (“WAT”) samples were collected, and gene expression was measured by qRT-PCR. Expression of glucose transporter 4 (GLUT4; FIG. 30A) and stearoyl-CoA-desaturase 1 (SCD1; FIG. 30B) was measured in adipose tissue, and expression of Glucose-6-phosphatase (G6P; FIG. 30C), Activating Transcription Factor 4 (ATF4; FIG. 30D), and 3-hydroxy-3-methylglutaryl (HMGCS; FIG. 30E) was measured in liver tissue. Data represent mean±SEM; statistical differences were determined using two tailed student's t-test.

### DETAILED DESCRIPTION

The invention is based, in part, upon the discovery that PTP1B interacts with the SH3-2 domain of the protein NCK1 to produce an intracellular signaling complex involved in the insulin receptor signaling pathway. NCK1 is a cytoplasmic adaptor protein known to be involved in transducing signals from receptor tyrosine kinases to downstream signaling recipients. NCK1 modulates the expression of PTP1B, and NCK1 depletion enhances insulin sensitivity similar to that seen with PTP1B inhibition (Li et al. (2014) supra). Deletion of NCK1 has previously been found to decrease ER stress, improve glucose tolerance and insulin signaling in mice (Latreille et al. (2011) AM. J. PHYSIOL. ENDOCRINOL. METAB. 300(3): E423-E434; Bywaters et al. (2021)) and to increase pancreatic β cells survival and insulin synthesis under diabetic conditions (Yamani et al. (2014) MOL. BIOL. CELL 25(5): 702-711; Yamani et al. (2015) CELL. SIGNAL. 27(12): 2555-2567). NCK1 has been found to play a role in regulating insulin signaling via activation of the PI3K/AKT pathway via a PTP1B-dependent mechanism, i.e. forming an interaction complex with PTP1B (Li et al. (2014) supra).

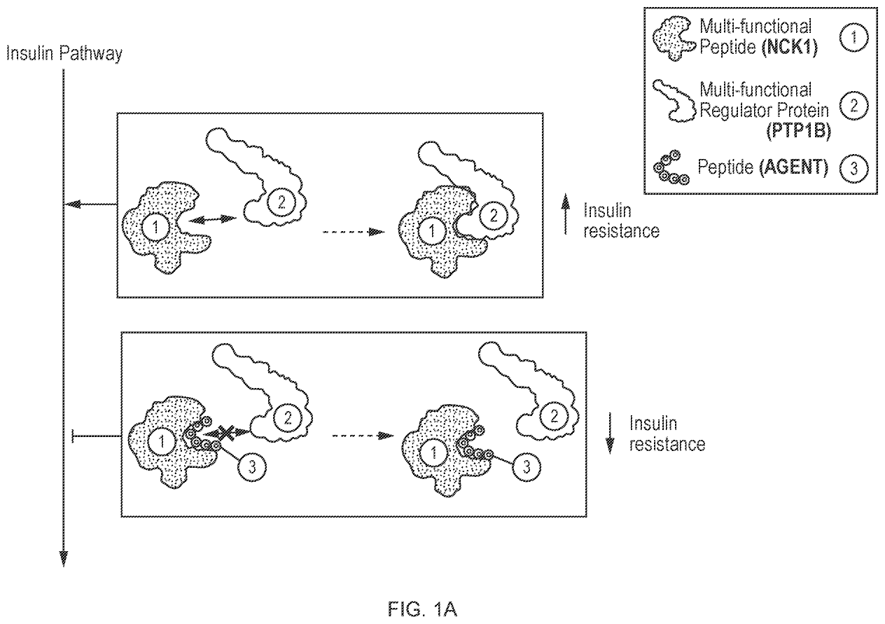

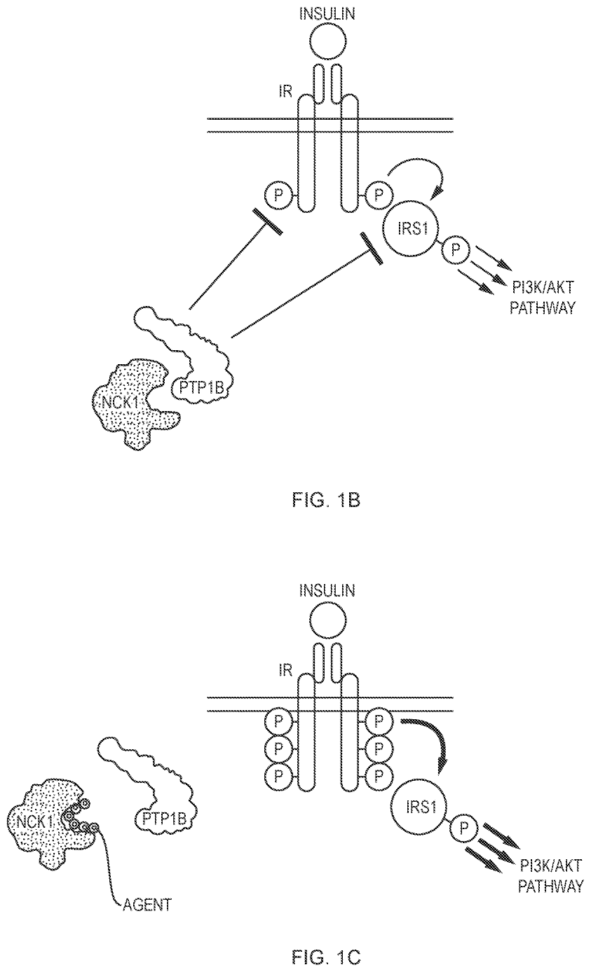

As discussed herein, it has been found that the interaction between the SH3-2 domain of NCK1 and PTP1B can modulate the activity of the insulin receptor (IR) and the IR-mediated pathway. By contacting NCK1 with an agent that interferes with the interaction between the NCK1 SH3-2 domain and PTP1B, PTP1B-mediated downregulation of the IR signaling pathway can be suppressed, thereby restoring IR signaling (see, FIGS. 1A-1C). Additionally, it is anticipated that interfering with the specific complex formation between NCK1 and PTP1B (as opposed to inhibiting PTP1B activity broadly) will not suppress other activities of PTP1B, which are important for normal cellular function. Likewise, by only targeting one domain of NCK1, it is anticipated that other important functions of the NCK1 adaptor protein will not be materially impacted. Disruption of the NCK1-PTP1B complex presents a novel approach to functionally achieving selective inhibition of PTP1B.

Accordingly, the disclosure provides, among other things, synthetic peptides that bind NCK1, and particularly the SH3-2 domain of NCK1. Furthermore, the NCK1-binding synthetic peptides of the disclosure can be administered to a subject in need thereof to treat or prevent a disease or disorder, e.g., diabetes or obesity.

### I. Definitions

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

The term “effective amount” as used herein refers to the amount of an active agent (e.g., a synthetic peptide) sufficient to effect beneficial or desired results. An effective amount can be administered in one or more administrations, applications, or dosages and is not intended to be limited to a particular formulation or administration route. For example, an effective amount may be an amount sufficient to achieve one or more of the following: (i) modulate an activity of NCK1, (ii) modulate an activity of PTP1B, (iii) inhibit, disrupt, reduce, or prevent NCK1-binding to PTP1B, (iv) increase, enhance, or restore insulin receptor signaling; (v) treat or prevent a disease or disorder, e.g., Type 2 diabetes or obesity, or a symptom or manifestation thereof; (vi) decrease body weight or lessen an increase in body weight; or (vii) decrease body fat mass or lessen an increase in body fat mass.

As used herein, the term “isolated” when used in conjunction with a particular article (e.g., protein, peptide, nucleic acid, or cell) is understood to mean: (1) that the article has been separated or purified from other components (e.g., other proteins, peptides, nucleic acids, cells, or cellular materials) and/or chemicals (e.g., reagents used in manufacture); (2) that the article may be separated or purified from the environment in which it may exist in nature, for example, a tissue or fluid sample; or (3) that the article does not occur in nature. For example, a molecule that is removed from a cell that produces it, is “isolated.” A chemically synthesized molecule is “isolated.” As used herein, the term “isolated” can also refer to a molecule that is substantially free of other molecules of the same species. For example, a protein or peptide may be “isolated” from other proteins or peptides having different amino acid sequences. The purity or homogeneity of a desired article can be assayed using techniques well known in the art, including gel electrophoresis, high performance liquid chromatography, or mass spectrometry. Any of the polynucleotides or polypeptides described herein may be isolated.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents.

The compositions also can include stabilizers and preservatives. For examples of carriers, stabilizers, and adjuvants, see e.g., Adeboye Adejare, Remington: The Science and Practice of Pharmacy (23d ed. 2020).

As used herein, “percent identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., nucleic acid sequence identity or amino acid sequence identity) can be achieved in various ways that are within the skill in the art, for instance, using publicly available computer software such as BLAST (Basic Local Alignment Search Tool), BLAST-2, ALIGN, MEGALIGN (DNASTAR), CLUSTALW, CLUSTAL OMEGA, or MUSCLE software. For a discussion of basic issues in searching sequence databases see Altschul et al., (1994) NATURE GENETICS 6:119-129, which is fully incorporated by reference herein. Those skilled in the art can determine appropriate parameters for aligning sequences, including any algorithms needed to achieve maximal alignment over the full length of the sequences being compared.

As used herein, “treat”, “treating”, and “treatment” refer to the treatment of a disease, disorder, or symptom or manifestation of such in a subject, e.g., in a human. This includes: (a) preventing a disease or disorder, (b) inhibiting the disease, disorder, etc., i.e., slowing or arresting its progress or development; and (b) relieving the disease, disorder, etc., i.e., causing regression of the disease state. As used herein, “prevent”, “preventing” and “prevention” refer to causing a disease, disorder, or symptom or manifestation of such not to occur for at least a period of time in at least some subjects. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

As used herein, the term “polypeptide”, refers to a polymer of amino acids, optionally including one or more amino acid analogs. A protein is a molecule composed of one or more polypeptides. A peptide is a relatively short polypeptide, typically between about 2 and 60 amino acids in length. However, depending upon the circumstances, the terms “protein”, “polypeptide”, and “peptide” may be used interchangeably. It is contemplated that polypeptides used herein may contain amino acids such as those that are naturally found in proteins, or can be replaced with amino acids that are not naturally found in proteins and/or amino acid analogs that are not amino acids, but where the resulting polypeptides have comparable binding activity or other biological activities as the polypeptides disclosed herein. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as an amide group or an acetyl group. For example, the polypeptide may be acetylated, e.g., at the N-terminus and/or amidated, e.g., at the C-terminus.

Polypeptides may, for example, be purified from natural sources, produced in vitro or in vivo using recombinant DNA technology in suitable expression systems (e.g., by recombinant host cells or in transgenic animals or plants), synthesized through chemical means such as solid phase peptide synthesis, liquid-phase peptide synthesis, and/or methods involving chemical ligation of synthesized peptides (see, e.g., Kent (2003) J PEPT. SCI. 9(9):574-93), or any combination of the foregoing. These methods are well known, and one of skill in the art will be able to select and implement an appropriate method for synthesizing the peptides and polypeptides described herein.

As used herein, the terms “polypeptide sequence” or “amino acid sequence” can refer to a sequence of covalently coupled amino acids, but is not necessarily restricted to the sequence information (i.e. the succession of letters or three letter codes chosen among the letters and codes used as abbreviations for amino acid names) that biochemically characterizes a polypeptide. A polypeptide sequence presented herein is presented in an N-terminal to C-terminal direction unless otherwise indicated.

The term “purified”, as used herein, means that an entity or substance is separated from one or more other entities or substances with which it was previously found before being purified. An entity or substance may be partially purified, substantially purified, or pure. A substance or entity such as a nucleic acid or polypeptide is considered pure when it is removed from substantially all other compounds or entities other than a solvent and any ions contained in the solvent, i.e., it constitutes at least about 90%, more preferably at least about 91%, 92%, 93%, 94%, 95%, 96%, 97%, 98%, 99%, or greater than 99% of the dry weight of the composition. A partially or substantially purified compound or entity such as a nucleic acid or polypeptide may be removed from at least 50%, at least 60%, at least 70%, or at least 80% by weight of the material with which it is naturally found, e.g., cellular material such as cellular proteins and/or nucleic acids. In certain embodiments, the purified nucleic acid or polypeptide constitutes at least 10%, 20%, 30%, 40%, 50%, 60%, 70%, 80%, 90%, 95%, 99% or even more, by dry weight, of the total nucleic acid or polypeptide, respectively, in a composition. Methods for assessing purity are known in the art and include chromatographic methods, immunological methods, electrophoretic methods, etc. Any of the polynucleotides or polypeptides described herein may be purified.

As used herein, “recombinant host cells”, “host cells”, and other such terms, denote prokaryotic or eukaryotic cells or cell lines that have been used as recipients for an exogenous nucleic acid (typically DNA) such as an expression vector in to which a nucleic acid portion that encodes a polypeptide of interest has been inserted. These terms include the progeny of the original cell in to which the vector or other nucleic acid has been introduced. Appropriate unicellular host cells include any of those routinely used in expressing polynucleotides (e.g., eukaryotic, mammalian, and/or viral polynucleotides) including, for example, prokaryotes, such as E. coli; and eukaryotes, including for example, fungi, such as yeast (e.g., Pichiapastoris and Saccharomyces cerevisiae); insect cells (e.g., Sf9), plant cells, and animal cells, e.g., mammalian cells such as CHO, R1.1, B-W, L-M, African Green Monkey Kidney cells (e.g. COS-1, COS-7, BSC-1, BSC-40 and BMT-10) and cultured human cells. Terms such as “host cells”, etc., are also used to refer to cells or cell lines that can be used as recipients for an exogenous nucleic acid, prior to its introduction. As used herein, the term “recombinant nucleic acid” refers to a nucleic acid that contains nucleic acid portions that are not found joined to gether in nature. As used herein, the term “recombinant polypeptide” refers to a polypeptide that is produced by transcription and translation of an exogenous nucleic acid by a recombinant host cell, typically after introduction of an expression vector that contains a portion that encodes the recombinant polypeptide in to the host cell.

As used herein, the term “charged amino acid” refers to an amino acid selected from arginine, histidine, lysine, aspartic acid (i.e., aspartate), and glutamic acid (i.e., glutamate). As used herein, the term “positively charged amino acid” refers to an amino acid selected from arginine, histidine, and lysine. As used herein, the term “negatively charged amino acid” refers to an amino acid selected from aspartic acid (i.e., aspartate) and glutamic acid (i.e., glutamate).

As used herein, the term “uncharged amino acid” refers to an amino acid selected from alanine, asparagine, cysteine, glutamine, glycine, isoleucine, leucine, methionine, phenylalanine, proline, serine, threonine, tryptophan, tyrosine, and valine.

As used herein, the term “hydrophobic amino acid” refers to an amino acid selected from alanine, cysteine, isoleucine, leucine, methionine, phenylalanine, tryptophan, and valine.

As used herein, the term “hydrophilic amino acid” refers to an amino acid selected from arginine, asparagine, aspartic acid, glutamine, glutamic acid, and lysine.

As used herein, the term “neutral amino acid” refers to an amino acid selected from glycine, histidine, proline, serine, threonine, and tyrosine.

As used herein, the term “aliphatic amino acid” refers to an amino acid selected from alanine, glycine, isoleucine, leucine, proline, and valine.

As used herein, the term “aromatic amino acid” refers to an amino acid selected from phenylalanine, tryptophan, and tyrosine. As used herein, the term “non-aromatic amino acid” refers to an amino acid selected from alanine, arginine, asparagine, aspartic acid, cysteine, glutamic acid, glutamine, glycine, histidine, isoleucine, leucine, lysine, methionine, proline, serine, threonine, and valine.

As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). In certain embodiments, a synthetic peptide of the disclosure comprises 1, 2, 3, 4, 5, 6, 7, 8, 9, 10, 11, 12, 13, 14, or 15 conservative substitutions relative to a “wild type” or reference peptide or amino acid sequence. In certain embodiments, a synthetic peptide of the disclosure comprises less than 2, less than 3, less than 4, less than 5, less than 6, less than 7, less than 8, less than 9, less than 10, less than 11, less than 12, less than 13, less than 14, or less than 15 conservative substitutions relative to a “wild type” or reference peptide or amino acid sequence.

As used herein, the term “carbonyl-containing moiety” refers to any chemical moiety that contains a carbonyl group. In certain embodiments, the carbonyl-containing moiety is not and does not contain an amino acid. A non-limiting example of a carbonyl-containing moiety is an acetyl group. In certain embodiments, the carbonyl group of the carbonyl-containing moiety is covalently attached to the most N-terminal nitrogen of the N-terminal amino acid in a peptide. In certain embodiments, if the N-terminal amino acid is proline, then the carbonyl group of the carbonyl-containing moiety is covalently attached to the nitrogen adjacent to the alpha carbon in proline's five-membered ring.

### II. Synthetic Peptides

The present disclosure encompasses the finding that synthetic peptides comprising a motif of PTP1B can bind NCK1. NCK1 is a cytoplasmic adaptor protein known to be involved in transducing signals from receptor tyrosine kinases to downstream signaling proteins. NCK1 comprises, in the N- to C-terminal direction, a first, second, and third SRC Homology 3 domain (SH3 domain; referred to herein as SH3-1, SH3-2, and SH3-3, respectively) and an SRC Homology 2 domain (SH2 domain). SH3 domains are small, intracellular protein domains which mediate protein-protein interactions. An exemplary amino acid sequence for wild-type human NCK1 is set forth as SEQ ID NO: 140. The SH3-1, SH3-2, and SH3-3 domains of NCK1 correspond to positions 2-61, 106-165, and 190-252, respectively, of SEQ ID NO: 140.

Without wishing to be bound by theory, it is believed that by binding to the SH3-2 domain of NCK1, the synthetic peptides comprising at least a portion of the PTP1B motif reduce, inhibit, or prevent interaction between PTP1B and NCK1, thereby specifically modulating the activity of PTP1B, e.g., by reducing or inhibiting PTP1B-mediated downregulation of insulin receptor signaling and/or by modulating regulation of the PI3K/AKT pathway.

Accordingly, in one aspect, the disclosure provides a synthetic NCK1-binding peptide. In certain embodiments, a synthetic NCK1-binding peptide of the disclosure comprises the amino acid sequence of SEQ ID NO: 1:

(SEQ ID NO: 1)
PX₁PPX₂R,

wherein X₁ and X₂ are each independently any amino acid. In certain embodiments, the peptide is 5 to 60 (e.g., 6 to 60), e.g., 5 to 50 (e.g., 6 to 50), e.g., 5 to 40 (e.g., 6 to 40) or 5 to 30 (e.g., 6 to 30), 5 to 20 (e.g., 6 to 20) or 5 to 10 (e.g., 6 to 10) consecutive amino acids in length. In certain embodiments, the peptide is 5 to 100 (e.g., 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100), 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive amino acids in length.

In certain embodiments, X₁ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₁ is a charged amino acid. In certain embodiments, X₁ a positively charged amino acid. For example, in certain embodiments, X₁ is arginine, histidine, or lysine. In certain embodiments, X₁ is arginine.

In certain embodiments, X₁ is a negatively charged amino acid. For example, in certain embodiments, X₁ is glutamic acid. In certain embodiments, X₁ is an uncharged amino acid. For example, in certain embodiments, X₁ is a hydrophobic amino acid. In certain embodiments, X₁ is selected from the group consisting of alanine, asparagine, and serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₂ is a charged amino acid. In certain embodiments, X₂ is a positively charged amino acid. For example, in certain embodiments, X₂ is arginine, histidine, or lysine. In certain embodiments, X₂ is lysine.

In certain embodiments, X₂ is a negatively charged amino acid. For example, in certain embodiments, X₂ is glutamic acid. In certain embodiments, X₂ is an uncharged amino acid. For example, in certain embodiments, X₂ is alanine, asparagine, cysteine, serine, or tryptophan. In certain embodiments, X₂ is a hydrophobic amino acid. For example, in certain embodiments, X₂ is alanine, cysteine, or tryptophan.

In certain embodiments, X₂ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₂ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₂ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₁ and X₂ are both charged amino acids. In certain embodiments, X₁ and X₂ are both positively charged amino acids. In certain embodiments, X₁ is arginine and X₂ is lysine.

In certain embodiments, the proline in position 1 in SEQ ID NO: 1 is the N-terminal amino acid of the synthetic peptide. In certain embodiments, the proline in position 1 is N-terminally modified by covalent linkage to a carbonyl-containing moiety, e.g., an acetyl moiety.

Without wishing to be bound by theory, the linkage to a carbonyl-containing moiety may enhance binding of the synthetic peptide to NCK1.

In certain embodiments, the proline in position 1 in SEQ ID NO: 1 is not the N-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to the N-terminus of the proline in position 1. In certain embodiments, each of the at least 1, at least 2, at least 3, etc., additional consecutive amino acids is, independently, any amino acid. In certain embodiments, the amino acid immediately N-terminal to position 1 is proline. In certain embodiments, the two amino acids immediately N-terminal to position 1 are both proline. In certain embodiments, the three amino acids immediately N-terminal to position 1 are all proline. In certain embodiments, the amino acids immediately N-terminal to position 1 are, in the N-to-C terminal direction: Pro-Pro-Glu-His-Ile-Pro-Pro-Pro (SEQ ID NO: 85).

In certain embodiments, the synthetic peptide further comprises an amino acid residue (X₀) that is N-terminal to position 1 in SEQ ID NO: 1. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the arginine in position 6 in SEQ ID NO: 1 is the C-terminal amino acid of the synthetic peptide. In certain embodiments, the arginine in position 6 is modified. In certain embodiments, the arginine in position 6 is the C-terminal amino acid of the synthetic peptide, and comprises an amide moiety at the C-terminus.

In certain embodiments, the arginine in position 6 in SEQ ID NO: 1 is not the C-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to the C-terminus of the arginine in position 6. In certain embodiments, each of the at least 1, at least 2, at least 3, etc., additional consecutive amino acids is, independently, any amino acid. In certain embodiments, the amino acid immediately C-terminal to position 6 is isoleucine. In certain embodiments, the two amino acids immediately C-terminal to position 6 are, in the N-to-C-terminal direction, isoleucine and leucine. In certain embodiments, the three amino acids immediately C-terminal to position 6 are, in the N-to-C-terminal direction, isoleucine, leucine, and glutamic acid.

In certain embodiments, the N-terminal amino acid is chemically modified (e.g., acetylated) and/or the C-terminal amino acid is chemically modified (e.g., amidated).

In certain embodiments, the N-terminal amino acid is acetylated and the C-terminal amino acid is amidated.

In certain embodiments, a synthetic NCK1-binding peptide of the disclosure comprises the amino acid sequence of SEQ ID NO: 2:

(SEQ ID NO: 2)
PX₁X₂PX₃R,

wherein X₁, X₂, and X₃ are each independently any amino acid. In certain embodiments, X₂ is a non-aromatic amino acid. In certain embodiments, the peptide is 5 to 60 (e.g., 6 to 60), e.g., 5 to 50 (e.g., 6 to 50), e.g., 5 to 40 (e.g., 6 to 40) or 5 to 30 (e.g., 6 to 30), 5 to 20 (e.g., 6 to 20) or 5 to 10 (e.g., 6 to 10) consecutive amino acids in length. In certain embodiments, the peptide is 5 to 100 (e.g., 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100), 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive amino acids in length.

In certain embodiments, X₁ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₁ is a charged amino acid. In certain embodiments, X₁ a positively charged amino acid. For example, in certain embodiments, X₁ is arginine, histidine, or lysine. In certain embodiments, X₁ is arginine.

In certain embodiments, X₁ is a negatively charged amino acid. For example, in certain embodiments, X₁ is glutamic acid. In certain embodiments, X₁ is an uncharged amino acid. For example, in certain embodiments, X₁ is a hydrophobic amino acid. In certain embodiments, X₁ is selected from the group consisting of alanine, asparagine, and serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is a charged amino acid, an uncharged amino acid, a hydrophobic amino acid, a hydrophilic amino acid, a neutral amino acid, an aliphatic amino acid, or an aromatic amino acid. In certain embodiments, X₂ is an uncharged amino acid. In certain embodiments, X₂ is an uncharged, non-aromatic amino acid. For example, in certain embodiments, X₂ is proline or alanine. In certain embodiments, X₂ is proline. In certain embodiments, X₂ is a hydrophobic amino acid. In certain embodiments, X₂ is a hydrophobic, non-aromatic amino acid, e.g., alanine. In certain embodiments, X₂ is a charged amino acid. In certain embodiments, X₂ is a positively charged amino acid, e.g., arginine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₃ is a charged amino acid. In certain embodiments, X₃ is a positively charged amino acid. For example, in certain embodiments, X₃ is arginine, histidine, or lysine. In certain embodiments, X₃ is lysine.

In certain embodiments, X₃ is a negatively charged amino acid. For example, in certain embodiments, X₃ is glutamic acid. In certain embodiments, X₃ is an uncharged amino acid. For example, in certain embodiments, X₃ is alanine, asparagine, cysteine, serine, or tryptophan. In certain embodiments, X₃ is a hydrophobic amino acid. For example, in certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₁ is arginine, X₂ is proline, and/or X₃ is lysine.

In certain embodiments, if X₁ is alanine and X₂ is valine, then X₃ is not proline. In certain embodiments, if X₂ is proline and X₃ is arginine, then X₁ is not arginine or glutamine. In certain embodiments, if X₂ is proline and X₃ is methionine, then X₁ is not histidine.

In certain embodiments, the proline in position 1 in SEQ ID NO: 2 is the N-terminal amino acid of the synthetic peptide. In certain embodiments, the proline in position 1 is N-terminally modified by covalent linkage to a carbonyl-containing moiety, e.g., an acetyl moiety.

In certain embodiments, the proline in position 1 in SEQ ID NO: 2 is not the N-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to the N-terminus of the proline in position 1. In certain embodiments, each of the at least 1, at least 2, at least 3, etc., additional consecutive amino acids is, independently, any amino acid. In certain embodiments, the amino acid immediately N-terminal to position 1 is proline. In certain embodiments, the two amino acids immediately N-terminal to position 1 are both proline. In certain embodiments, the three amino acids immediately N-terminal to position 1 are all proline. In certain embodiments, the amino acids immediately N-terminal to position 1 are, in the N-to-C terminal direction: Pro-Pro-Glu-His-Ile-Pro-Pro-Pro (SEQ ID NO: 85).

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the arginine in position 6 in SEQ ID NO: 2 is the C-terminal amino acid of the synthetic peptide. In certain embodiments, the arginine in position 6 is modified. In certain embodiments, the arginine in position 6 is the C-terminal amino acid and comprises an amide moiety at the C-terminus.

In certain embodiments, the arginine in position 6 in SEQ ID NO: 2 is not the C-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to the C-terminus of the arginine in position 6. In certain embodiments, each of the at least 1, at least 2, at least 3, etc., additional consecutive amino acids is, independently, any amino acid. In certain embodiments, the amino acid immediately C-terminal to position 6 is isoleucine. In certain embodiments, the two amino acids immediately C-terminal to position 6 are, in the N-to-C-terminal direction, isoleucine and leucine. In certain embodiments, the three amino acids immediately C-terminal to position 6 are, in the N-to-C-terminal direction, isoleucine, leucine, and glutamic acid.

In certain embodiments, a synthetic NCK1-binding peptide of the disclosure comprises the amino acid sequence of SEQ ID NO: 3:

(SEQ ID NO: 3)
PX₁X₂PX₃RX₄,

wherein X₁, X₂, and X₃, are each independently any amino acid, and wherein X₄ is either absent or any amino acid. In certain embodiments, X₂ is a non-aromatic amino acid. In certain embodiments, the peptide is 5 to 60 (e.g., 6 to 60), e.g., 5 to 50 (e.g., 6 to 50), e.g., 5 to 40 (e.g., 6 to 40) or 5 to 30 (e.g., 6 to 30), 5 to 20 (e.g., 6 to 20) or 5 to 10 (e.g., 6 to 10) consecutive amino acids in length. In certain embodiments, the peptide is 5 to 100 (e.g., 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100), 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive amino acids in length.

In certain embodiments, X₁ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₁ is a charged amino acid. In certain embodiments, X₁ a positively charged amino acid. For example, in certain embodiments, X₁ is arginine, histidine, or lysine. In certain embodiments, X₁ is arginine.

In certain embodiments, X₁ is a negatively charged amino acid. For example, in certain embodiments, X₁ is glutamic acid. In certain embodiments, X₁ is an uncharged amino acid. For example, in certain embodiments, X₁ is a hydrophobic amino acid. In certain embodiments, X₁ is selected from the group consisting of alanine, asparagine, and serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is a charged amino acid, an uncharged amino acid, a hydrophobic amino acid, a hydrophilic amino acid, a neutral amino acid, an aliphatic amino acid, or a non-aromatic amino acid. In certain embodiments, X₂ is an uncharged amino acid. In certain embodiments, X₂ is an uncharged, non-aromatic amino acid, e.g., proline or alanine. In certain embodiments, X₂ is proline. In certain embodiments, X₂ is a hydrophobic amino acid. In certain embodiments, X₂ is a hydrophobic, non-aromatic amino acid, e.g., alanine. In certain embodiments, X₂ is a charged amino acid. In certain embodiments, X₂ is a positively charged amino acid, e.g., arginine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₃ is a charged amino acid. In certain embodiments, X₃ is a positively charged amino acid. For example, in certain embodiments, X₃ is arginine, histidine, or lysine. In certain embodiments, X₃ is lysine.

In certain embodiments, X₃ is a negatively charged amino acid. For example, in certain embodiments, X₃ is glutamic acid. In certain embodiments, X₃ is an uncharged amino acid. For example, in certain embodiments, X₃ is alanine, asparagine, cysteine, serine, or tryptophan. In certain embodiments, X₃ is a hydrophobic amino acid. For example, in certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent. In certain embodiments, X₄ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₄ is an uncharged amino acid. In certain embodiments, X₄ is a hydrophobic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is arginine, X₂ is proline, X₃ is lysine, and/or X₄ is absent.

In certain embodiments wherein X₂ is proline and X₄ is not absent: (i) X₁ is not threonine; (ii) X₃ is not lysine; and/or (iii) X₄ is not serine. In certain embodiments wherein X₂ is proline and X₄ is not absent: (i) X₁ is not arginine; (ii) X₃ is not threonine; and/or (iii) X₄ is not threonine. In certain embodiments wherein X₁ is alanine and X₂ is valine, X₃ is not proline. In certain embodiments wherein X₂ is proline and X₄ is absent, (i) X₁ is not arginine and/or X₃ is not serine. In certain embodiments wherein X₂ is proline and X₄ is not absent: (i) if X₁ is arginine, X₃ is lysine, and X₄ is isoleucine, then X₄ is not the C-terminal amino acid; (ii) if X₄ is alanine, then X₁ is not proline and/or X₃ is not arginine; and/or (iii) if X₄ is proline, then: (a) if X₁ is methionine, then X₃ is not asparagine; and (b) if X₁ is arginine, then X₃ is not glycine.

In certain embodiments, the proline in position 1 in SEQ ID NO: 3 is the N-terminal amino acid of the synthetic peptide. In certain embodiments, the proline in position 1 is N-terminally modified by covalent linkage to a carbonyl-containing moiety, e.g., an acetyl moiety.

In certain embodiments, the proline in position 1 in SEQ ID NO: 3 is not the N-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to the N-terminus of the proline in position 1. In certain embodiments, each of the at least 1, at least 2, at least 3, etc., additional consecutive amino acids is, independently, any amino acid. In certain embodiments, the amino acid immediately N-terminal to position 1 is proline. In certain embodiments, the two amino acids immediately N-terminal to position 1 are both proline. In certain embodiments, the three amino acids immediately N-terminal to position 1 are all proline. In certain embodiments, the amino acids immediately N-terminal to position 1 are, in the N-to-C terminal direction: Pro-Pro-Glu-His-Ile-Pro-Pro-Pro (SEQ ID NO: 85).

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the arginine in position 6 in SEQ ID NO: 3 is the C-terminal amino acid of the synthetic peptide. In certain embodiments, the arginine in position 6 is modified. In certain embodiments, the arginine in position 6 is the C-terminal amino acid and comprises an amide moiety at the C-terminus.

In certain embodiments wherein X₄ is present, X₄ in SEQ ID NO: 3 is the C-terminal amino acid of the synthetic peptide. In certain embodiments, X₄ is modified. In certain embodiments, X₄ is the C-terminal amino acid and comprises an amide moiety at the C-terminus.

In certain embodiments wherein X₄ is present, X₄ in SEQ ID NO: 3 is not the C-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond)C-terminally to X₄. In certain embodiments, each of the at least 1, at least 2, at least 3, etc., additional consecutive amino acids is, independently, any amino acid. In certain embodiments, the amino acid immediately C-terminal to X₄ is leucine. In certain embodiments, the two amino acids immediately C-terminal to X₄ are, in the N-to-C-terminal direction, leucine and glutamic acid.

In certain embodiments, a synthetic NCK1-binding peptide of the disclosure comprises the amino acid sequence of SEQ ID NO: 4:

(SEQ ID NO: 4)
PRPPKRX₁

wherein X₁ is either absent or any amino acid. In certain embodiments, the peptide is 5 to 60 (e.g., 6 to 60), e.g., 5 to 50 (e.g., 6 to 50), e.g., 5 to 40 (e.g., 6 to 40) or 5 to 30 (e.g., 6 to 30), 5 to 20 (e.g., 6 to 20) or 5 to 10 (e.g., 6 to 10) consecutive amino acids in length. In certain embodiments, the peptide is 5 to 100 (e.g., 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100), 15 to 100, 20 to 100, 25 to 100, 30 to 100, 35 to 100, 40 to 100, 45 to 100, 50 to 100, 55 to 100, 60 to 100, 65 to 100, 70 to 100, 75 to 100, 80 to 100, 85 to 100, 90 to 100, or 95 to 100 consecutive amino acids in length.

In certain embodiments, X₁ is absent. In certain embodiments, X₁ is a charged amino acid, an uncharged amino acid, a hydrophobic amino acid, a hydrophilic amino acid, a neutral amino acid, an aliphatic amino acid, an aromatic amino acid, or a non-aromatic amino acid. In certain embodiments, X₁ is an uncharged amino acid. In certain embodiments, X₁ is a hydrophobic amino acid. In certain embodiments, X₁ in SEQ ID NO: 4 is a positively charged amino acid, e.g., an amino acid selected from the group consisting of arginine, histidine, and lysine. In certain embodiments, X₁ is a negatively charged amino acid, e.g., an amino acid selected from the group consisting of aspartic acid and glutamic acid. In certain embodiments, X₁ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₁ is an aliphatic amino acid, e.g., an amino acid selected from the group consisting of alanine, glycine, isoleucine, leucine, proline, and valine. In certain embodiments, X₁ is isoleucine. In certain embodiments, X₁ is an aromatic amino acid, e.g., an amino acid selected from the group consisting of phenylalanine, tryptophan, and tyrosine. In certain embodiments, X₁ is a sulfur-containing amino acid, e.g., an amino acid selected from the group consisting of cysteine and methionine. In certain embodiments, X₁ is an amino acid with a hydroxyl-containing side chain, e.g., an amino acid selected from the group consisting of serine and threonine. In certain embodiments, X₁ is an acidic amino acid, e.g., an amino acid selected from the group consisting of aspartic acid and glutamic acid. In certain embodiments, X₁ is a basic amino acid, e.g., an amino acid selected from the group consisting of arginine, histidine, and lysine. In certain embodiments, X₁ is an amino acid with an amide-containing side chain, e.g., an amino acid selected from the group consisting of asparagine and glutamine.

In certain embodiments wherein X₁ is isoleucine, X₁ is not the C-terminal amino acid.

In certain embodiments, the proline in position 1 in SEQ ID NO: 4 is the N-terminal amino acid of the synthetic peptide. In certain embodiments, the proline in position 1 is N-terminally modified by covalent linkage to a carbonyl-containing moiety, e.g., an acetyl moiety.

In certain embodiments, the proline in position 1 in SEQ ID NO: 4 is not the N-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to the N-terminus of the proline in position 1. In certain embodiments, each of the at least 1, at least 2, at least 3, etc., additional consecutive amino acids is, independently, any amino acid. In certain embodiments, the amino acid immediately N-terminal to position 1 is proline. In certain embodiments, the two amino acids immediately N-terminal to position 1 are both proline. In certain embodiments, the three amino acids immediately N-terminal to position 1 are all proline. In certain embodiments, the amino acids immediately N-terminal to position 1 are, in the N-to-C terminal direction: Pro-Pro-Glu-His-Ile-Pro-Pro-Pro (SEQ ID NO: 85).

In certain embodiments, the synthetic peptide further comprises an amino acid residue (X₀) that is N-terminal to position 1 in SEQ ID NO: 4. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments wherein X₁ is absent, the arginine in position 6 in SEQ ID NO: 4 is the C-terminal amino acid of the synthetic peptide. In certain embodiments, the arginine in position 6 is modified. In certain embodiments, the arginine in position 6 is the C-terminal peptide and comprises an amide moiety at the C-terminus.

In certain embodiments wherein X₁ is present, X₁ in SEQ ID NO: 4 is the C-terminal amino acid of the synthetic peptide. In certain embodiments, X₁ is modified. In certain embodiments, X₁ is the C-terminal peptide and comprises an amide moiety at the C-terminus.

In certain embodiments wherein X₁ is present, X₁ in SEQ ID NO: 4 is not the C-terminal amino acid of the synthetic peptide. For example, in certain embodiments, the synthetic peptide comprises at least 1, at least 2, at least 3, at least 4, at least 5, at least 6, at least 7, at least 8, at least 9, or at least 10 or more additional consecutive amino acids covalently linked (e.g., linked by peptide bond) to C-terminally to X₁. In certain embodiments, each of the at least 1, at least 2, at least 3, etc., additional consecutive amino acids is, independently, any amino acid. In certain embodiments, the amino acid immediately C-terminal to X₁ is leucine. In certain embodiments, the two amino acids immediately C-terminal to X₁ are, in the N-to-C-terminal direction, leucine and glutamic acid.

In certain embodiments, a synthetic peptide of the disclosure comprises the amino acid sequence of a NCK1-binding fragment of PTP1B. For example, in certain embodiments, the synthetic peptide comprises, consists essentially of, or consists of, the amino acid sequence of any one of SEQ ID NOs: 5-21, 135-137, or 141-143. In certain embodiments, the synthetic peptide comprises, consists essentially of, or consists of, the amino acid sequence of SEQ ID NO: 8. In certain embodiments, the synthetic peptide comprises, consists essentially of, or consists of, the amino acid sequence of SEQ ID NO: 9. In certain embodiments, the synthetic peptide comprises, consists essentially of, or consists of, the amino acid sequence of SEQ ID NO: 10.

TABLE 1
Exemplary NCK1-binding fragments of PTP1B

| Peptide Sequence | SEQ ID NO: |
|---|---|
| PRPPKRI | 5 |
| PRPPKRIL | 6 |
| PRPPKRILE | 7 |
| PRPPKR | 8 |
| PPRPPKR | 9 |
| PPPEHIPPPPRPPKR | 10 |
| PPPRPPKR | 11 |
| PPPPRPPKR | 12 |
| IPPPPRPPKR | 13 |
| HIPPPPRPPKR | 14 |
| EHIPPPPRPPKR | 15 |
| PEHIPPPPRPPKR | 16 |
| PPEHIPPPPRPPKR | 17 |
| PPRPPKRI | 18 |
| PPRPPKRIL | 19 |
| PPRPPKRILE | 20 |
| PPPEHIPPPPRPPKRILE | 21 |
| PPPEHIPPPPRPPKRILEPH | 135 |
| HEDLEPPPEHIPPPPRPPKR | 136 |
| PPPEHIPPPPRPPKRILEPHNGKCR | 137 |
| PPPEHIPPPPRPPKRILEPHNGKCREFFPN | 141 |
| WKELSHEDLEPPPEHIPPPPRPPKR | 142 |
| SVQDQWKELSHEDLEPPPEHIPPPPRPPKR | 143 |

In certain embodiments, a synthetic peptide of the disclosure comprises, consists essentially of, or consists of, the amino acid sequence of any of SEQ ID NOs: 1-4, and the amino acid sequence of the synthetic peptide is at least 5000 identical to the amino acid sequence of any one of SEQ ID NOs: 5-21, 135-137, or 141-143. For example, in certain embodiments, the amino acid sequence of the synthetic peptide is at least 50%, at least 55%, at least 60%, at least 65%, at least 70%, at least 7500, at least 80%, at least 85%, at least 90 00, or at least 95% identical to the amino acid sequences of any one of SEQ ID NOs: 5-21, 135-137, or 141-143. In certain embodiments, the synthetic peptide comprises the amino acid sequence of any of SEQ ID NOs 1-4, and the amino acid sequence of the synthetic peptide is at least 50%, at least 55%, at least 60%, at least 65%, at least 70%, at least 75%, at least 80%, at least 85%, at least 90%, or at least 95% identical to the amino acid sequence of SEQ ID NO: 8. In certain embodiments, the synthetic peptide comprises the amino acid sequence of any of SEQ ID NOs 1-4, and the amino acid sequence of the synthetic peptide is at least 50%, at least 55%, at least 60%, at least 65%, at least 70%, at least 75%, at least 80%, at least 85%, at least 90%, or at least 95% identical to the amino acid sequence of SEQ ID NO: 9. In certain embodiments, the synthetic peptide comprises the amino acid sequence of any of SEQ ID NOs 1-4, and the amino acid sequence of the synthetic peptide is at least 50%, at least 55%, at least 60%, at least 65%, at least 70%, at least 75%, at least 80%, at least 85%, at least 90%, or at least 95% identical to the amino acid sequence of SEQ ID NO: 10. In certain embodiments, the synthetic peptide comprises a conservative substitution relative to any of the amino acid sequences of SEQ ID NOs: 5-21, 135-137, or 141-143, e.g., SEQ ID NO: 8, SEQ ID NO: 9, or SEQ ID NO: 10. In certain embodiments, the synthetic peptide comprises less than 2, less than 3, less than 4, less than 5, less than 6, less than 7, less than 8, less than 9, less than 10, less than 11, less than 12, or less than 13 conservative substitutions relative to the amino acid sequence of any one of SEQ ID NOs: 5-21, 135-137, or 141-143, e.g., SEQ ID NO: 8, SEQ ID NO: 9, or SEQ ID NO: 10. In certain embodiments, the synthetic peptide comprises 1, 2, 3, 4, 5, 6, 7, 8, 9, 10, 11, or 12 or more conservative substitutions relative to the amino acid sequence of any one of SEQ ID NOs: 5-21, 135-137, or 141-143, e.g., SEQ ID NO: 8, SEQ ID NO: 9, or SEQ ID NO: 10.

In certain embodiments, a synthetic peptide of the disclosure comprises, or consists essentially of, or consists of the amino acid sequence of any one of SEQ ID NOs: 5-30, 32-33, 35, 38, 41, 59, 61-83, 87-89, 98, 100-137, 141-158, or 168. In certain embodiments, a synthetic peptide of the disclosure comprises, consists essentially of, or consists of the amino acid sequence of any one of the peptides set forth in Table 2.

TABLE 2
Exemplary NCK1-binding peptides

| Peptide Sequence | SEQ ID NO: |
|---|---|
| PRPPKRI | 5 |
| PRPPKRIL | 6 |
| PRPPKRILE | 7 |
| PRPPKR | 8 |
| PPRPPKR | 9 |
| PPPEHIPPPPRPPKR | 10 |
| PPPRPPKR | 11 |
| PPPPRPPKR | 12 |
| IPPPPRPPKR | 13 |
| HIPPPPRPPKR | 14 |
| EHIPPPPRPPKR | 15 |
| PEHIPPPPRPPKR | 16 |
| PPEHIPPPPRPPKR | 17 |
| PPRPPKRI | 18 |
| PPRPPKRIL | 19 |
| PPRPPKRILE | 20 |
| PPPEHIPPPPRPPKRILE | 21 |
| APPEHIPPPPRPPKR | 22 |
| PAPEHIPPPPRPPKR | 23 |
| PPAEHIPPPPRPPKR | 24 |
| PPPAHIPPPPRPPKR | 25 |
| PPPEAIPPPPRPPKR | 26 |
| PPPEHAPPPPRPPKR | 27 |
| PPPEHIAPPPRPPKR | 28 |
| PPPEHIPAPPRPPKR | 29 |
| PPPEHIPPAPRPPKR | 30 |
| PPPEHIPPPPAPPKR | 32 |
| PPPEHIPPPPRAPKR | 33 |
| PPPEHIPPPPRPPAR | 35 |
| PAPPKR | 38 |
| PRPPAR | 41 |
| APRPPKR | 59 |
| RPRPPKR | 61 |
| NPRPPKR | 62 |
| CPRPPKR | 63 |
| EPRPPKR | 64 |
| HPRPPKR | 65 |
| FPRPPKR | 66 |
| SPRPPKR | 67 |
| PPKPPKR | 68 |
| PPFPPKR | 69 |
| PPHPPKR | 70 |
| PPEPPKR | 71 |
| PPSPPKR | 72 |
| PPNPPKR | 73 |
| PPCPPKR | 74 |
| PPRRPKR | 75 |
| PPRFPKR | 76 |
| PPRPPRR | 77 |
| PPRPPHR | 78 |
| PPRPPWR | 79 |
| PPRPPER | 80 |
| PPRPPSR | 81 |
| PPRPPNR | 82 |
| PPRPPCR | 83 |
| PPRIPKR | 87 |
| PPRLPKR | 88 |
| PPRKPKR | 89 |
| PPRTPKR | 98 |
| PPRYPKR | 100 |
| PPRVPKR | 101 |
| PPRPPGR | 102 |
| PPRPPIR | 103 |
| PPRPPLR | 104 |
| PPRPPFR | 105 |
| PPRPPDR | 106 |
| PPRPPMR | 107 |
| PPRPPQR | 108 |
| PPRPPPR | 109 |
| PPRPPTR | 110 |
| PPRPPYR | 111 |
| PPRPPVR | 112 |
| PPDPPKR | 113 |
| PPQPPKR | 114 |
| PPGPPKR | 115 |
| PPLPPKR | 116 |
| PPIPPKR | 117 |
| PPPPPKR | 118 |
| PPMPPKR | 119 |
| PPTPPKR | 120 |
| PPWPPKR | 121 |
| PPYPPKR | 122 |
| PPVPPKR | 123 |
| DPRPPKR | 124 |
| QPRPPKR | 125 |
| GPRPPKR | 126 |
| IPRPPKR | 127 |
| LPRPPKR | 128 |
| KPRPPKR | 129 |
| MPRPPKR | 130 |
| TPRPPKR | 131 |
| WPRPPKR | 132 |
| YPRPPKR | 133 |
| VPRPPKR | 134 |
| PPPEHIPPPPRPPKRILEPH | 135 |
| HEDLEPPPEHIPPPPRPPKR | 136 |
| PPPEHIPPPPRPPKRILEPHNGKCR | 137 |
| PPPEHIPPPPRPPKRILEPHNGKCREFFPN | 141 |
| WKELSHEDLEPPPEHIPPPPRPPKR | 142 |
| SVQDQWKELSHEDLEPPPEHIPPPPRPPKR | 143 |
| PPRPPKRA | 144 |
| PPRPPKRR | 145 |
| PPRPPKRN | 146 |
| PPRPPKRD | 147 |
| PPRPPKRC | 148 |
| PPRPPKRF | 149 |
| PPRPPKRS | 150 |
| PPRPPKRG | 151 |
| PPRPPKRH | 152 |
| PPRPPKRL | 153 |
| PPRPPKRK | 154 |
| PPRPPKRM | 155 |
| PPRPPKRP | 156 |
| PPRPPKRW | 157 |
| PPRPPKRY | 158 |
| H-Pyr-PRPPKR | 168 |

In certain embodiments, a synthetic peptide of the disclosure comprises, consists essentially of, or consists of, the amino acid sequence of any of SEQ ID NOs: 1-4, and the amino acid sequence of the synthetic peptide is at least 50% identical to the amino acid sequence of any one of SEQ ID NOs: 5-30, 32-33, 35, 38, 41, 59, 61-83, 87-89, 98, 100-137, 141-158, or 168. For example, in certain embodiments, the amino acid sequence of the synthetic peptide is at least 50%, at least 55%, at least 60%, at least 65%, at least 70%, at least 75%, at least 80%, at least 85%, at least 90%, or at least 95% identical to the amino acid sequences of any one of SEQ ID NOs: 5-30, 32-33, 35, 38, 41, 59, 61-83, 87-89, 98, 100-137, 141-158, or 168. In certain embodiments, the synthetic peptide comprises a conservative substitution relative to any of the amino acid sequences of SEQ ID NOs: 5-30, 32-33, 35, 38, 41, 59, 61-83, 87-89, 98, 100-137, 141-158, or 168. In certain embodiments, the synthetic peptide comprises less than 2, less than 3, less than 4, less than 5, less than 6, less than 7, less than 8, less than 9, less than 10, less than 11, less than 12, or less than 13 conservative substitutions relative to the amino acid sequence of any one of SEQ ID NOs: 5-30, 32-33, 35, 38, 41, 59, 61-83, 87-89, 98, 100-137, 141-158, or 168. In certain embodiments, the synthetic peptide comprises 1, 2, 3, 4, 5, 6, 7, 8, 9, 10, 11, or 12 or more conservative substitutions relative to the amino acid sequence of any one of SEQ ID NOs: 5-30, 32-33, 35, 38, 41, 59, 61-83, 87-89, 98, 100-137, 141-158, or 168.

In certain embodiments, a synthetic peptide of the disclosure comprises at least 3 proline residues. For example, in certain embodiments, the synthetic peptide comprises at least 3, at least 4, at least 5, at least 6, at least 7, at least 9, or at least 10 proline residues. In certain embodiments, the synthetic peptide has 1, 2, 3, 4, 5, 6, 7, 8, 9, or 10 proline residues.

In certain embodiments, a synthetic peptide of the disclosure comprises at least 2 positively charged amino acids. For example, in certain embodiments, a synthetic peptide of the disclosure comprises at least 2, at least 3, at least 4, at least 5, at least 6, at least 7, at least 8, at least 9, or at least 10 positively charged amino acids. In certain embodiments, the synthetic peptide has 1, 2, 3, 4, 5, 6, 7, 8, 9, or 10 positively charged amino acid residues.

In certain embodiments, it is contemplated that a synthetic peptide of the disclosure can consist essentially of (or consist of) 5 to 100 (e.g., 6 to 100), 5 to 95 (e.g., 6 to 95), 5 to 90 (e.g. 6 to 90), 5 to 85 (e.g., 6 to 85), 5 to 80 (e.g., 6 to 80), 5 to 75 (e.g., 6 to 75), 5 to 70 (e.g., 6 to 70), 5 to 65 (e.g., 6 to 65), 5 to 60 (e.g., 6 to 60), e.g., 5 to 50 (e.g., 6 to 50), e.g., 5 to 40 (e.g., 6 to 40) or 5 to 30 (e.g., 6 to 30), 5 to 20 (e.g., 6 to 20) or 5 to 10 (e.g., 6 to 10) consecutive amino acids. For example, in certain embodiments, the synthetic peptide consists essentially of (or consists of) 5 to 100, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 6 to 100, 6 to 95, 6 to 90, 6 to 85, 6 to 80, 6 to 75, 6 to 70, 6 to 65, 6 to 60, 6 to 55, 6 to 50, 6 to 45, 6 to 40, 6 to 35, 6 to 30, 6 to 25, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 100, 7 to 95, 7 to 90, 7 to 85, 7 to 80, 7 to 75, 7 to 70, 7 to 65, 7 to 60, 7 to 55, 7 to 50, 7 to 45, 7 to 40, 7 to 35, 7 to 30, 7 to 25, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 100, 8 to 95, 8 to 90, 8 to 85, 8 to 80, 8 to 75, 8 to 70, 8 to 65, 8 to 60, 8 to 55, 8 to 50, 8 to 45, 8 to 40, 8 to 35, 8 to 30, 8 to 25, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 100, 9 to 95, 9 to 90, 9 to 85, 9 to 80, 9 to 75, 9 to 70, 9 to 65, 9 to 60, 9 to 55, 9 to 50, 9 to 45, 9 to 40, 9 to 35, 9 to 30, 9 to 25, 9 to 20, 9 to 19, 9 to 18, 9 to 17, 9 to 16, 9 to 15, 9 to 14, 9 to 13, 9 to 12, 9 to 11, 9 to 10, 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 19, 10 to 18, 10 to 17, 10 to 16, 10 to 15, 10 to 14, 10 to 13, 10 to 12, 10 to 11, 11 to 100, 11 to 95, 11 to 90, 11 to 85, 11 to 80, 11 to 75, 11 to 70, 11 to 65, 11 to 60, 11 to 55, 11 to 50, 11 to 45, 11 to 40, 11 to 35, 11 to 30, 11 to 25, 11 to 20, 11 to 19, 11 to 18, 11 to 17, 11 to 16, 11 to 15, 11 to 14, 11 to 13, 11 to 12, 12 to 100, 12 to 95, 12 to 90, 12 to 85, 12 to 80, 12 to 75, 12 to 70, 12 to 65, 12 to 60, 12 to 55, 12 to 50, 12 to 45, 12 to 40, 12 to 35, 12 to 30, 12 to 25, 12 to 20, 12 to 19, 12 to 18, 12 to 17, 12 to 16, 12 to 15, 12 to 14, 12 to 13, 13 to 100, 13 to 95, 13 to 90, 13 to 85, 13 to 80, 13 to 75, 13 to 70, 13 to 65, 13 to 60, 13 to 55, 13 to 50, 13 to 45, 13 to 40, 13 to 35, 13 to 30, 13 to 25, 13 to 20, 13 to 19, 13 to 18, 13 to 17, 13 to 16, 13 to 15, 13 to 14, 14 to 100, 14 to 95, 14 to 90, 14 to 85, 14 to 80, 14 to 75, 14 to 70, 14 to 65, 14 to 60, 14 to 55, 14 to 50, 14 to 45, 14 to 40, 14 to 35, 14 to 30, 14 to 25, 14 to 20, 14 to 19, 14 to 18, 14 to 17, 14 to 16, 14 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 3515 to 30, 15 to 25, 15 to 20, 15 to 19, 15 to 18, 15 to 17, 15 to 16, 16 to 100, 16 to 95, 16 to 90, 16 to 85, 16 to 80, 16 to 75, 16 to 70, 16 to 65, 16 to 60, 16 to 55, 16 to 50, 16 to 45, 16 to 40, 16 to 35, 16 to 30, 16 to 25, 16 to 20, 16 to 19, 16 to 18, 16 to 17, 17 to 100, 17 to 95, 17 to 90, 17 to 85, 17 to 80, 17 to 75, 17 to 70, 17 to 65, 17 to 60, 17 to 55, 17 to 50, 17 to 45, 17 to 40, 17 to 35, 17 to 30, 17 to 25, 17 to 20, 17 to 19, 17 to 18, 18 to 100, 18 to 95, 18 to 90, 18 to 85, 18 to 80, 18 to 75, 18 to 70, 18 to 65, 18 to 60, 18 to 55, 18 to 50, 18 to 45, 18 to 40, 18 to 35, 18 to 30, 18 to 25, 18 to 20, 18 to 19, 19 to 100, 19 to 95, 19 to 90, 19 to 85, 19 to 80, 19 to 75, 19 to 70, 19 to 65, 19 to 60, 19 to 55, 19 to 50, 19 to 45, 19 to 40, 19 to 35, 19 to 30, 19 to 25, 19 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 consecutive amino acids. In certain embodiments, the synthetic peptide consists essentially of (or consists of) 6 to 15 consecutive amino acids.

In certain embodiments, a synthetic peptide of the disclosure consists essentially of (or consists of) an amino acid sequence of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 consecutive amino acids. In certain embodiments, the synthetic peptide consists essentially of (or consists of) 6, 7, or 15 consecutive amino acids.

In certain embodiments, a synthetic peptide of the disclosure is 5 to 100 (e.g., 5 to 60, e.g., 5 to 30) amino acids in length. For example, in certain embodiments, the synthetic peptide is 5 to 100, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, 6 to 100, 6 to 95, 6 to 90, 6 to 85, 6 to 80, 6 to 75, 6 to 70, 6 to 65, 6 to 60, 6 to 55, 6 to 50, 6 to 45, 6 to 40, 6 to 35, 6 to 30, 6 to 25, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, 6 to 7, 7 to 100, 7 to 95, 7 to 90, 7 to 85, 7 to 80, 7 to 75, 7 to 70, 7 to 65, 7 to 60, 7 to 55, 7 to 50, 7 to 45, 7 to 40, 7 to 35, 7 to 30, 7 to 25, 7 to 20, 7 to 19, 7 to 18, 7 to 17, 7 to 16, 7 to 15, 7 to 14, 7 to 13, 7 to 12, 7 to 11, 7 to 10, 7 to 9, 7 to 8, 8 to 100, 8 to 95, 8 to 90, 8 to 85, 8 to 80, 8 to 75, 8 to 70, 8 to 65, 8 to 60, 8 to 55, 8 to 50, 8 to 45, 8 to 40, 8 to 35, 8 to 30, 8 to 25, 8 to 20, 8 to 19, 8 to 18, 8 to 17, 8 to 16, 8 to 15, 8 to 14, 8 to 13, 8 to 12, 8 to 11, 8 to 10, 8 to 9, 9 to 100, 9 to 95, 9 to 90, 9 to 85, 9 to 80, 9 to 75, 9 to 70, 9 to 65, 9 to 60, 9 to 55, 9 to 50, 9 to 45, 9 to 40, 9 to 35, 9 to 30, 9 to 25, 9 to 20, 9 to 19, 9 to 18, 9 to 17, 9 to 16, 9 to 15, 9 to 14, 9 to 13, 9 to 12, 9 to 11, 9 to 10, 10 to 100, 10 to 95, 10 to 90, 10 to 85, 10 to 80, 10 to 75, 10 to 70, 10 to 65, 10 to 60, 10 to 55, 10 to 50, 10 to 45, 10 to 40, 10 to 35, 10 to 30, 10 to 25, 10 to 20, 10 to 19, 10 to 18, 10 to 17, 10 to 16, 10 to 15, 10 to 14, 10 to 13, 10 to 12, 10 to 11, 11 to 100, 11 to 95, 11 to 90, 11 to 85, 11 to 80, 11 to 75, 11 to 70, 11 to 65, 11 to 60, 11 to 55, 11 to 50, 11 to 45, 11 to 40, 11 to 35, 11 to 30, 11 to 25, 11 to 20, 11 to 19, 11 to 18, 11 to 17, 11 to 16, 11 to 15, 11 to 14, 11 to 13, 11 to 12, 12 to 100, 12 to 95, 12 to 90, 12 to 85, 12 to 80, 12 to 75, 12 to 70, 12 to 65, 12 to 60, 12 to 55, 12 to 50, 12 to 45, 12 to 40, 12 to 35, 12 to 30, 12 to 25, 12 to 20, 12 to 19, 12 to 18, 12 to 17, 12 to 16, 12 to 15, 12 to 14, 12 to 13, 13 to 100, 13 to 95, 13 to 90, 13 to 85, 13 to 80, 13 to 75, 13 to 70, 13 to 65, 13 to 60, 13 to 55, 13 to 50, 13 to 45, 13 to 40, 13 to 35, 13 to 30, 13 to 25, 13 to 20, 13 to 19, 13 to 18, 13 to 17, 13 to 16, 13 to 15, 13 to 14, 14 to 100, 14 to 95, 14 to 90, 14 to 85, 14 to 80, 14 to 75, 14 to 70, 14 to 65, 14 to 60, 14 to 55, 14 to 50, 14 to 45, 14 to 40, 14 to 35, 14 to 30, 14 to 25, 14 to 20, 14 to 19, 14 to 18, 14 to 17, 14 to 16, 14 to 15, 15 to 100, 15 to 95, 15 to 90, 15 to 85, 15 to 80, 15 to 75, 15 to 70, 15 to 65, 15 to 60, 15 to 55, 15 to 50, 15 to 45, 15 to 40, 15 to 3515 to 30, 15 to 25, 15 to 20, 15 to 19, 15 to 18, 15 to 17, 15 to 16, 16 to 100, 16 to 95, 16 to 90, 16 to 85, 16 to 80, 16 to 75, 16 to 70, 16 to 65, 16 to 60, 16 to 55, 16 to 50, 16 to 45, 16 to 40, 16 to 35, 16 to 30, 16 to 25, 16 to 20, 16 to 19, 16 to 18, 16 to 17, 17 to 100, 17 to 95, 17 to 90, 17 to 85, 17 to 80, 17 to 75, 17 to 70, 17 to 65, 17 to 60, 17 to 55, 17 to 50, 17 to 45, 17 to 40, 17 to 35, 17 to 30, 17 to 25, 17 to 20, 17 to 19, 17 to 18, 18 to 100, 18 to 95, 18 to 90, 18 to 85, 18 to 80, 18 to 75, 18 to 70, 18 to 65, 18 to 60, 18 to 55, 18 to 50, 18 to 45, 18 to 40, 18 to 35, 18 to 30, 18 to 25, 18 to 20, 18 to 19, 19 to 100, 19 to 95, 19 to 90, 19 to 85, 19 to 80, 19 to 75, 19 to 70, 19 to 65, 19 to 60, 19 to 55, 19 to 50, 19 to 45, 19 to 40, 19 to 35, 19 to 30, 19 to 25, 19 to 20, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25, 25 to 100, 25 to 95, 25 to 90, 25 to 85, 25 to 80, 25 to 75, 25 to 70, 25 to 65, 25 to 60, 25 to 55, 25 to 50, 25 to 45, 25 to 40, 25 to 35, 25 to 30, 30 to 100, 30 to 95, 30 to 90, 30 to 85, 30 to 80, 30 to 75, 30 to 70, 30 to 65, 30 to 60, 30 to 55, 30 to 50, 30 to 45, 30 to 40, 30 to 35, 35 to 100, 35 to 95, 35 to 90, 35 to 85, 35 to 80, 35 to 75, 35 to 70, 35 to 65, 35 to 60, 35 to 55, 35 to 50, 35 to 45, 35 to 40, 40 to 100, 40 to 95, 40 to 90, 40 to 85, 40 to 80, 40 to 75, 40 to 70, 40 to 65, 40 to 60, 40 to 55, 40 to 50, 40 to 45, 45 to 100, 45 to 95, 45 to 90, 45 to 85, 45 to 80, 45 to 75, 45 to 70, 45 to 65, 45 to 60, 45 to 55, 45 to 50, 50 to 100, 50 to 95, 50 to 90, 50 to 85, 50 to 80, 50 to 75, 50 to 70, 50 to 65, 50 to 60, 50 to 55, 55 to 100, 55 to 95, 55 to 90, 55 to 85, 55 to 80, 55 to 75, 55 to 70, 55 to 65, 55 to 60, 60 to 100, 60 to 95, 60 to 90, 60 to 85, 60 to 80, 60 to 75, 60 to 70, 60 to 65, 65 to 100, 65 to 95, 65 to 90, 65 to 85, 65 to 80, 65 to 75, 65 to 70, 70 to 100, 70 to 95, 70 to 90, 70 to 85, 70 to 80, 70 to 75, 75 to 100, 75 to 95, 75 to 90, 75 to 85, 75 to 80, 80 to 100, 80 to 95, 80 to 90, 80 to 85, 85 to 100, 85 to 95, 85 to 90, 90 to 100, 90 to 95, or 95 to 100 amino acids in length. In certain embodiments, the synthetic peptide is 6 to 15 amino acids in length.

In certain embodiments, the synthetic peptide is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 consecutive amino acids in length. In certain embodiments, the synthetic peptide is 6, 7, or 15 amino acids in length.

In certain embodiments, a synthetic peptide of the disclosure comprises at least one or at least two amino acid residues that are covalently linked to a chemical moiety, e.g., a moiety not naturally found in a naturally occurring peptide. In certain embodiments, the N-terminal and/or the C-terminal amino acid of the synthetic peptide is modified by the addition of a chemical moiety, e.g., a moiety not naturally found in a naturally occurring peptide. In certain embodiments, the N-terminal amino acid is modified by the addition of an acetyl group or a pyroglutamic acid. In certain embodiments, the N-terminal amino acid is acetylated. In certain embodiments, the C-terminal amino acid is modified by the addition of an amide group or an ethylamide group. In certain embodiments, the C-terminal amino acid is amidated.

In certain embodiments, the K_{D} value with which a synthetic peptide of the disclosure binds NCK1 is less than or equal to 1 mM, 900 μM, 800 μM, 700 μM, 600 μM, 500 μM, 450 μM, 400 μM, 350 μM, 300 μM, 250 μM, 200 μM, 175 μM, 150 μM, 125 μM, 100 μM, 90 μM, 80 μM, 70 μM, 60 μM, or 50 μM, for example, as determined by isothermal titration calorimetry (ITC). In certain embodiments, the K_{D} value with which a synthetic peptide of the disclosure binds the SH3-2 domain of NCK1 is less than or equal to 1 mM, 900 μM, 800 μM, 700 μM, 600 μM, 500 μM, 450 μM, 400 μM, 350 μM, 300 μM, 250 μM, 200 μM, 175 μM, 150 μM, 125 μM, 100 μM, 90 μM, 80 μM, 70 μM, 60 μM, or 50 μM, for example, as determined by ITC.

In certain embodiments, a synthetic peptide of the disclosure is a 7-mer peptide comprising or consisting essentially of the amino acid sequence of SEQ ID NO: 9, wherein the N-terminus is acetylated and the and the C-terminus is in amide form. In certain embodiments, the molecular formula of the peptide is C₄₀H₆₉N₁₅O₈, and/or the molecular weight of the peptide is 888.1 g/mol. In certain embodiments, the synthetic peptide is formulated as a bis-acetate salt, optionally wherein the molecular formula of the bis-acetate salt is C₄₄H₇₇N₁₅O₁₂ and/or the molecular weight of the bis-acetate salt is 1008.2 g/mol. In certain embodiments, the synthetic peptide is formulated as a tri-acetate salt, optionally wherein the molecular formula of the tri-acetate salt is C₄₆H₈₁N₁₅O₁₄ and/or the molecular weight of the tri-acetate salt is 1068.2 g/mol.

a. Six- to 30-mer or Six to 50-mer Synthetic Peptides with PXPPXR Motif (SEQ ID NO: 1)

In certain embodiments described herein, the disclosure relates to a synthetic peptide consisting essentially of 6 to 30 (e.g., 6 to 15, 6 to 16, 6 to 17, 6 to 18, 6 to 19, 6 to 20, 6 to 21, 6 to 22, 6 to 23, 6 to 24, 6 to 25, 6 to 26, 6 to 27, 6 to 28, 6 to 29, 6 to 30), or 6 to 50 (e.g., 6 to 15, 6 to 16, 6 to 17, 6 to 18, 6 to 19, 6 to 20, 6 to 21, 6 to 22, 6 to 23, 6 to 24, 6 to 25, 6 to 26, 6 to 27, 6 to 28, 6 to 29, 6 to 30, 6 to 31, 6 to 32, 6 to 33, 6 to 34, 6 to 35, 6 to 36, 6 to 37, 6 to 38, 6 to 39, 6 to 40, 6 to 41, 6 to 42, 6 to 43, 6 to 44, 6 to 45, 6 to 46, 6 to 47, 6 to 48, 6 to 49 or 6 to 50) consecutive amino acids, including an N-terminal amino acid and a C-terminal amino acid, wherein the synthetic peptide comprises the amino acid sequence of SEQ ID NO 1:

(SEQ ID NO: 1)
PX₁PPX₂R,

wherein X₁ and X₂ are each independently any amino acid, wherein: (i) the proline in position 1 of SEQ ID NO: 1 is the N-terminal amino acid; (ii) the N-terminal amino acid is chemically modified (e.g., acetylated) and/or the C-terminal amino acid is chemically modified (e.g., amidated); and/or (iii) the C-terminal amino acid is arginine provided that, (1) if X₂ is arginine, then X₁ is not arginine or glutamine, (2) if X₂ is serine, X₁ is not arginine, and (3) if X₂ is methionine, then X₁ is not histidine. In certain embodiments wherein the proline in position 1 is the N-terminal amino acid, the proline in position 1 is modified with a chemical moiety (e.g., by covalent linkage to a carbonyl-containing moiety, e.g., an acetyl moiety).

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is a charged amino acid, for example a positively charged amino acid or a negatively charged amino acid. In certain embodiments, X₂ is arginine, histidine, or lysine. In certain embodiments, X₂ is lysine. In certain embodiments, X₂ is glutamic acid. In certain embodiments, X₂ is an uncharged amino acid. In certain embodiments, X₂ is alanine, asparagine, cysteine, serine, or tryptophan. In certain embodiments, X₂ is a hydrophobic amino acid. In certain embodiments, X₂ is alanine, cysteine, or tryptophan.

In certain embodiments, X₂ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₂ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₂ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₁ and X₂ are both charged amino acids. In certain embodiments, X₁ and X₂ are both positively charged amino acids. In certain embodiments, X₁ and X₂ are each independently selected from arginine, histidine, and lysine. In certain embodiments, X₁ is arginine and X₂ is lysine.

In certain embodiments, the synthetic peptide further comprises an amino acid residue (X₀) that is N-terminal to position 1 in SEQ ID NO: 1. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

b. Seven- to Ten-mer Synthetic Peptides with PXXPXR Motif (SEQ ID NO: 2)

In certain embodiments described herein, the disclosure relates to a synthetic peptide consisting essentially of an amino acid sequence of 7 to 10 consecutive amino acids, including an N-terminal amino acid and a C-terminal amino acid, wherein the synthetic peptide comprises the amino acid sequence of SEQ ID NO: 2:

(SEQ ID NO: 2)
PX₁X₂PX₃R

wherein: (i) X₁ and X₃ are each independently any amino acid, and X₂ is a non-aromatic amino acid; (ii) if X₁ is alanine and X₂ is valine, then X₃ is not proline; (iii) if X₂ is proline and X₃ is arginine, then X₁ is not arginine or glutamine; and (iv) if X₂ is proline and X₃ is methionine, then X₁ is not histidine. In certain embodiments wherein the proline at position 1 of SEQ ID NO: 2 is the N-terminal amino acid of the synthetic peptide, the amino acid is modified with a chemical moiety (e.g., covalent linkage to a carbonyl-containing moiety, e.g., an acetyl moiety.

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₁ and X₃ are both charged amino acids. In certain embodiments, X₁ and X₃ are both positively charged amino acids. In certain embodiments, X₁ and X₃ are each independently selected from arginine, histidine, and lysine. In certain embodiments, X₁ is arginine and X₃ is lysine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine.

In certain embodiments, X₁ is a charged amino acid, e.g., a positively charged amino acid; X₂ is an uncharged amino acid, e.g., a hydrophobic amino acid; and/or X₃ is a charged amino acid, e.g., a positively charged amino acid. In certain embodiments, X₁ is arginine, histidine, or lysine; X₂ is alanine or proline; and/or X₃ is arginine, histidine, or lysine. In certain embodiments, X₁ is arginine, X₂ is proline, and X₃ is lysine.

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of 7 to 10 consecutive amino acids, including an N-terminal amino acid and a C-terminal amino acid, wherein the synthetic peptide comprises the amino acid sequence of SEQ ID NO: 2:

(SEQ ID NO: 2)
PX₁X₂PX₃R

wherein: (i) X₁ and X₃ are each independently any amino acid, and X₂ is selected from the group consisting of alanine, arginine, isoleucine, leucine, lysine, phenylalanine, proline, threonine, tyrosine, and valine; (ii) if X₁ is alanine and X₂ is valine, then X₃ is not proline; (iii) if X₂ is proline and X₃ is arginine, then X₁ is not arginine or glutamine; and (iv) if X₂ is proline and X₃ is methionine, then X₁ is not histidine. In certain embodiments wherein the proline at position 1 of SEQ ID NO: 2 is the N-terminal amino acid of the synthetic peptide, the amino acid is modified with a chemical moiety (e.g., by covalent linkage to a carbonyl-containing moiety, e.g., an acetyl moiety).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

c. 11-mer Synthetic Peptides with PXXPXRX Motif (SEQ ID NO: 3)

In certain embodiments described herein, the disclosure relates to a synthetic peptide consisting essentially of an amino acid sequence of eleven consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (a) X₁ and X₃ are each independently any amino acid, X₂ is a non-aromatic amino acid, and X₄ is either absent or any amino acid, and (b) wherein if X₂ is proline and X₄ is not absent, then: (i) X₁ is not threonine; (ii) X₃ is not lysine; and/or (iii) X₄ is not serine. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is a charged amino acid, e.g., a positively charged amino acid; and X₃ is a charged amino acid, e.g., a positively charged amino acid. In certain embodiments, X₁ and X₃ are each independently selected from arginine, histidine, and lysine. In certain embodiments, X₁ is arginine and X₃ is lysine. In certain embodiments of the foregoing peptides, X₄ is absent or X₄ is isoleucine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline. In certain embodiments of the foregoing peptides, X₄ is absent or X₄ is isoleucine.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine. In certain embodiments of the foregoing peptides, X₄ is absent or X₄ is isoleucine.

In certain embodiments, X₁ is a charged amino acid, e.g., a positively charged amino acid; X₂ is an uncharged amino acid, e.g., a hydrophobic amino acid; X₃ is a charged amino acid, e.g., a positively charged amino acid; and/or X₄ is absent. In certain embodiments, X₁ is arginine, histidine, or lysine; X₂ is alanine or proline; X₃ is arginine, histidine, or lysine; and X₄ is absent. In certain embodiments, X₁ is arginine, X₂ is proline, X₃ is lysine, and X₄ is absent.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of eleven consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (a) X₁ and X₃ are each independently any amino acid; (b) X₂ is selected from the group consisting of alanine, arginine isoleucine, leucine, lysine, phenylalanine, proline, threonine, tyrosine, and valine; and (c) X₄ is either absent or any amino acid, and (d) wherein if X₂ is proline and X₄ is not absent, then: (i) X₁ is not threonine; (ii) X₃ is not lysine; and/or (iii) X₄ is not serine. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

d. 12-mer Synthetic Peptides with PXXPXRX Motif (SEQ ID NO: 3)

In certain embodiments described herein, the disclosure relates to a synthetic peptide consisting essentially of an amino acid sequence of 12 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (a) X₁ and X₃ are each independently any amino acid, X₂ is a non-aromatic amino acid, and X₄ is either absent or any amino acid, and (b) wherein if X₂ is proline and X₄ is not absent, then (i) X₁ is not arginine; (ii) X₃ is not threonine; and/or (iii) X₄ is not threonine. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is a charged amino acid, e.g., a positively charged amino acid; X₃ is a charged amino acid, e.g., a positively charged amino acid. In certain embodiments, X₁ and X₃ are each independently selected from arginine, histidine, and lysine. In certain embodiments, X₁ is arginine and X₃ is lysine. In certain embodiments of the foregoing peptides, X₄ is absent or X₄ is isoleucine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline. In certain embodiments of the foregoing peptides, X₄ is absent or X₄ is isoleucine.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine. In certain embodiments of the foregoing peptides, X₄ is absent or X₄ is isoleucine.

In certain embodiments, X₁ is a charged amino acid, e.g., a positively charged amino acid; X₂ is an uncharged amino acid, e.g., a hydrophobic amino acid; X₃ is a charged amino acid, e.g., a positively charged amino acid; and/or X₄ is absent. In certain embodiments, X₁ is arginine, histidine, or lysine; X₂ is alanine or proline; X₃ is arginine, histidine, or lysine; and X₄ is absent. In certain embodiments, X₁ is arginine, X₂ is proline, X₃ is lysine, and X₄ is absent.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of 12 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (a) X₁ and X₃ are each independently any amino acid; (b) X₂ is selected from the group consisting of alanine, arginine, isoleucine, leucine, lysine, phenylalanine, proline, threonine, tyrosine, and valine; (c) X₄ is either absent or any amino acid, and (d) wherein if X₂ is proline and X₄ is not absent, then (i) X₁ is not arginine; (ii) X₃ is not threonine; and/or (iii) X₄ is not threonine. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

e. 13-mer Synthetic Peptides with PXXPXRX Motif (SEQ ID NO: 3)

In certain embodiments described herein, the disclosure relates to a synthetic peptide consisting essentially of an amino acid sequence of 13 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein X₁ and X₃ are each independently any amino acid, X₂ is a non-aromatic amino acid, and X₄ is either absent or any amino acid. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is a charged amino acid, e.g., a positively charged amino acid; X₃ is a charged amino acid, e.g., a positively charged amino acid. In certain embodiments, X₁ and X₃ are each independently selected from arginine, histidine, and lysine. In certain embodiments, X₁ is arginine and X₃ is lysine. In certain embodiments of the foregoing peptides, X₄ is absent or X₄ is isoleucine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline. In certain embodiments of the foregoing peptides, X₄ is absent or X₄ is isoleucine.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine. In certain embodiments of the foregoing peptides, X₄ is absent or X₄ is isoleucine.

In certain embodiments, X₁ is a charged amino acid, e.g., a positively charged amino acid; X₂ is an uncharged amino acid, e.g., a hydrophobic amino acid; X₃ is a charged amino acid, e.g., a positively charged amino acid; and/or X₄ is absent. In certain embodiments, X₁ is arginine, histidine, or lysine; X₂ is alanine or proline; X₃ is arginine, histidine, or lysine; and X₄ is absent. In certain embodiments, X₁ is arginine, X₂ is proline, X₃ is lysine, and X₄ is absent.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of 13 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein X₁ and X₃ are each independently any amino acid, X₂ is selected from the group consisting of alanine, arginine isoleucine, leucine, lysine, phenylalanine, proline, threonine, tyrosine, and valine, and X₄ is either absent or any amino acid. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

f. 14-mer Synthetic Peptides with PXXPXRX Motif (SEQ ID NO: 3)

In certain embodiments described herein, the disclosure relates to a synthetic peptide consisting essentially of an amino acid sequence of 14 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (1) X₁ and X₃ are each independently any amino acid, X₂ is a non-aromatic amino acid, and X₄ is either absent or any amino acid; (2) if X₁ is alanine and X₂ is valine, then X₃ is not proline; and (3) if X₂ is proline and X₄ is absent, then: (i) X₁ is not arginine; and/or (ii) X₃ is not serine. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is a charged amino acid, e.g., a positively charged amino acid; X₃ is a charged amino acid, e.g., a positively charged amino acid. In certain embodiments, X₁ and X₃ are each independently selected from arginine, histidine, and lysine. In certain embodiments, X₁ is arginine and X₃ is lysine. In certain embodiments of the foregoing peptides, X₄ is absent or X₄ is isoleucine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline. In certain embodiments of the foregoing peptides, X₄ is absent or X₄ is isoleucine.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine. In certain embodiments of the foregoing peptides, X₄ is absent or X₄ is isoleucine.

In certain embodiments, X₁ is a charged amino acid, e.g., a positively charged amino acid; X₂ is an uncharged amino acid, e.g., a hydrophobic amino acid; X₃ is a charged amino acid, e.g., a positively charged amino acid; and/or X₄ is absent. In certain embodiments, X₁ is arginine, histidine, or lysine; X₂ is alanine or proline; X₃ is arginine, histidine, or lysine; and X₄ is absent. In certain embodiments, X₁ is arginine, X₂ is proline, X₃ is lysine, and X₄ is absent.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of 14 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (1) X₁ and X₃ are each independently any amino acid; (2) X₂ is selected from the group consisting of alanine, arginine, isoleucine, leucine, lysine, phenylalanine, proline, threonine, tyrosine, and valine; (3) if X₁ is alanine and X₂ is valine, then X₃ is not proline; and (4) if X₂ is proline and X₄ is absent, then: (i) X₁ is not arginine; and/or (ii) X₃ is not serine. In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

g. 15-mer Synthetic Peptides with PXXPXRX Motif (SEQ ID NO: 3)

In certain embodiments described herein, the disclosure relates to a synthetic peptide consisting essentially of an amino acid sequence of 15 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (1) X₁ and X₃ are each independently any amino acid, X₂ is a non-aromatic amino acid, and X₄ is either absent or any amino acid; and (2) wherein if X₂ is proline and X₄ is not absent, then:
   (i) if X₁ is arginine, X₃ is lysine, and X₄ is isoleucine, then X₄ is not the C-terminal amino acid and/or the N-terminal amino acid is not a proline;
   (ii) if X₄ is alanine, then X₁ is not proline and/or X₃ is not arginine; and
   (iii) if X₄ is proline, then:
      (a) if X₁ is methionine, then X₃ is not asparagine; and
      (b) if X₁ is arginine, then X₃ is not glycine.
         In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is a charged amino acid, e.g., a positively charged amino acid; X₃ is a charged amino acid, e.g., a positively charged amino acid. In certain embodiments, X₁ and X₃ are each independently selected from arginine, histidine, and lysine. In certain embodiments, X₁ is arginine and X₃ is lysine. In certain embodiments of the foregoing peptides, X₄ is absent or X₄ is isoleucine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline. In certain embodiments of the foregoing peptides, X₄ is absent or X₄ is isoleucine.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine. In certain embodiments of the foregoing peptides, X₄ is absent or X₄ is isoleucine.

In certain embodiments, X₁ is a charged amino acid, e.g., a positively charged amino acid; X₂ is an uncharged amino acid, e.g., a hydrophobic amino acid; X₃ is a charged amino acid, e.g., a positively charged amino acid; and/or X₄ is absent. In certain embodiments, X₁ is arginine, histidine, or lysine; X₂ is alanine or proline; X₃ is arginine, histidine, or lysine; and X₄ is absent. In certain embodiments, X₁ is arginine, X₂ is proline, X₃ is lysine, and X₄ is absent.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of 15 consecutive amino acids, including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (1) X₁ and X₃ are each independently any amino acid, (2) X₂ is selected from the group consisting of alanine, arginine, isoleucine, leucine, lysine, phenylalanine, proline, threonine, tyrosine, and valine, and (3) X₄ is either absent or any amino acid; and (4) wherein if X₂ is proline and X₄ is not absent, then:
   (i) if X₁ is arginine, X₃ is lysine, and X₄ is isoleucine, then X₄ is not the C-terminal amino acid and/or the N-terminal amino acid is not a proline;
   (ii) if X₄ is alanine, then X₁ is not proline and/or X₃ is not arginine; and
   (iii) if X₄ is proline, then:
      (a) if X₁ is methionine, then X₃ is not asparagine; and
      (b) if X₁ is arginine, then X₃ is not glycine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

h. 16- to 30-mer or 16- to 50-mer Synthetic Peptides with PXXPXRX Motif (SEQ ID NO: 3)

In certain embodiments described herein, the disclosure relates to a synthetic peptide consisting essentially of an amino acid sequence of (i) sixteen, seventeen, eighteen, nineteen, twenty, twenty-one, twenty-two, twenty-three, twenty-four, twenty-five, twenty-six, twenty-seven, twenty-eight, twenty-nine, or thirty consecutive amino acids, or (ii) sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty consecutive amino acids, each including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (1) X₁ and X₃ are each independently any amino acid, X₂ is a non-aromatic amino acid, and X₄ is either absent or any amino acid; and (2) wherein, if the synthetic peptide consists essentially of twenty consecutive amino acids, then the four most C-terminal amino acid residues of the synthetic peptide is not SLSR (SEQ ID NO: 85). In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments wherein the proline at position 1 of SEQ ID NO: 3 is the N-terminal amino acid of the synthetic peptide, the N-terminal amino acid is modified by covalent linkage to a carbonyl-containing moiety, e.g., an acetyl moiety.

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, X₁ is a charged amino acid, e.g., a positively charged amino acid; X₃ is a charged amino acid, e.g., a positively charged amino acid. In certain embodiments, X₁ and X₃ are each independently selected from arginine, histidine, and lysine. In certain embodiments, X₁ is arginine and X₃ is lysine. In certain embodiments of the foregoing peptides, X₄ is absent or X₄ is isoleucine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline. In certain embodiments of the foregoing peptides, X₄ is absent or X₄ is isoleucine.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine. In certain embodiments of the foregoing peptides, X₄ is absent or X₄ is isoleucine.

In certain embodiments, X₁ is a charged amino acid, e.g., a positively charged amino acid; X₂ is an uncharged amino acid, e.g., a hydrophobic amino acid; X₃ is a charged amino acid, e.g., a positively charged amino acid; and/or X₄ is absent. In certain embodiments, X₁ is arginine, histidine, or lysine; X₂ is alanine or proline; X₃ is arginine, histidine, or lysine; and X₄ is absent. In certain embodiments, X₁ is arginine, X₂ is proline, X₃ is lysine, and X₄ is absent.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments described herein, the disclosure relates to a synthetic peptide consisting essentially of an amino acid sequence of (i) sixteen, seventeen, eighteen, nineteen, twenty, twenty-one, twenty-two, twenty-three, twenty-four, twenty-five, twenty-six, twenty-seven, twenty-eight, twenty-nine, or thirty consecutive amino acids, or (ii) sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty consecutive amino acids, each including an N-terminal amino acid and a C-terminal amino acid, wherein the synthetic peptide comprises the amino acid sequence of SEQ ID NO: 3:

(SEQ ID NO: 3)
PX₁X₂PX₃RX₄,

wherein: (1) X₁ and X₃ are each independently any amino acid; (2) X₂ is selected from the group consisting of alanine, arginine, isoleucine, leucine, lysine, phenylalanine, proline, threonine, tyrosine, and valine; (3) X₄ is either absent or any amino acid; and (4) wherein, if the synthetic peptide consists essentially of twenty consecutive amino acids, then the four most C-terminal amino acid residues of the synthetic peptide is not SLSR (SEQ ID NO: 85). In certain embodiments wherein the proline at position 1 of SEQ ID NO: 3 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₄ is absent and the arginine in position 6 of SEQ ID NO: 3 is the C-terminal amino acid.

In certain embodiments, X₄ is present and is selected from any amino acid. In certain embodiments, X₄ is a charged amino acid, an uncharged amino acid, a hydrophobic amino acid, a hydrophilic amino acid, and/or a non-aromatic amino acid. In certain embodiments, X₄ is a positively charged amino acid, e.g., an amino acid selected from the group consisting of arginine, histidine, and lysine. In certain embodiments, X₄ is a negatively charged amino acid, e.g., an amino acid selected from the group consisting of aspartic acid and glutamic acid. In certain embodiments, X₄ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₄ is an aliphatic amino acid, e.g., an amino acid selected from the group consisting of alanine, glycine, isoleucine, leucine, proline, and valine. In certain embodiments, X₄ is isoleucine. In certain embodiments, X₄ is an aromatic amino acid, e.g., an amino acid selected from the group consisting of phenylalanine, tryptophan, and tyrosine. In certain embodiments, X₄ is a sulfur-containing amino acid, e.g., an amino acid selected from the group consisting of cysteine and methionine. In certain embodiments, X₄ is an amino acid with a hydroxyl-containing side chain, e.g., an amino acid selected from the group consisting of serine and threonine. In certain embodiments, X₄ is an acidic amino acid, e.g., an amino acid selected from the group consisting of aspartic acid and glutamic acid. In certain embodiments, X₄ is a basic amino acid, e.g., an amino acid selected from the group consisting of arginine, histidine, and lysine. In certain embodiments, X₄ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 3. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

i. Six- to 30-mer or Six- to 50-mer Synthetic Peptides with PRPPKRX Motif (SEQ ID NO: 4)

In certain embodiments described herein, the disclosure relates to a synthetic peptide consisting essentially of an amino acid sequence of (i) 6 to 30, e.g., 6 to 29, 6 to 28, 6 to 27, 6 to 26, 6 to 25, 6 to 24, 6 to 23, 6 to 22, 6 to 21, 6 to 20, 6 to 19, 6 to 18, 6 to 17, 6 to 16, or 6 to 15 consecutive amino acids or (ii) 6 to 50, e.g., 6 to 49, 6 to 48, 6 to 47, 6 to 46, 6 to 45, 6 to 44, 6 to 43, 6 to 42, 6 to 41, 6 to 40, 6 to 39, 6 to 38, 6 to 37, 6 to 36, 6 to 35, 6 to 34, 6 to 33, 6 to 32, 6 to 31, 6 to 30, 6 to 29, 6 to 28, 6 to 27, 6 to 26, 6 to 25, 6 to 24, 6 to 23, 6 to 22, 6 to 21, 6 to 20, 6 to 19, 6 to 18, 6 to 17, 6 to 16, or 6 to 15 consecutive amino acids, each including an N-terminal amino acid and a C-terminal amino acid, wherein the synthetic peptide comprises the amino acid sequence of SEQ ID NO: 4:

(SEQ ID NO: 4)
PRPPKRX₁,

wherein: (1) X₁ is absent or any amino acid, and (2) if X₁ is isoleucine and the synthetic peptide is a 15-mer, then X₁ is not the C-terminal amino acid and/or proline is not the N-terminal amino acid. In certain embodiments wherein the proline at position 1 of SEQ ID NO: 4 is the N-terminal amino acid of the synthetic peptide, the N-terminal amino acid is modified with a chemical moiety (e.g., by covalent linkage to a carbonyl-containing moiety, e.g., an acetyl moiety).

In certain embodiments, X₁ is absent and the arginine in position 6 of SEQ ID NO: 4 is the C-terminal amino acid. In certain embodiments, X₁ is present and is selected from any amino acid. In certain embodiments, X₁ is a charged amino acid, an uncharged amino acid, a hydrophobic amino acid, a hydrophilic amino acid, and/or a non-aromatic amino acid. In certain embodiments, X₁ is a positively charged amino acid, e.g., an amino acid selected from the group consisting of arginine, histidine, and lysine. In certain embodiments, X₁ is a negatively charged amino acid, e.g., an amino acid selected from the group consisting of aspartic acid and glutamic acid. In certain embodiments, X₁ is an uncharged amino acid, e.g., an amino acid selected from the group consisting of alanine, asparagine, cysteine, glycine, isoleucine, leucine, methionine, phenylalanine, proline, serine, threonine, tryptophan, tyrosine, and valine. In certain embodiments, X₁ is an aliphatic amino acid, e.g., an amino acid selected from the group consisting of alanine, glycine, isoleucine, leucine, proline, and valine. In certain embodiments, X₁ is isoleucine. In certain embodiments, X₁ is an aromatic amino acid, e.g., an amino acid selected from the group consisting of phenylalanine, tryptophan, and tyrosine. In certain embodiments, X₁ is a sulfur-containing amino acid, e.g., an amino acid selected from the group consisting of cysteine and methionine. In certain embodiments, X₁ is an amino acid with a hydroxyl-containing side chain, e.g., an amino acid selected from the group consisting of serine and threonine. In certain embodiments, X₁ is an acidic amino acid, e.g., an amino acid selected from the group consisting of aspartic acid and glutamic acid. In certain embodiments, X₁ is a basic amino acid, e.g., an amino acid selected from the group consisting of arginine, histidine, and lysine. In certain embodiments, X₁ is an amino acid with an amide-containing side chain, e.g., an amino acid selected from the group consisting of asparagine and glutamine.

In certain embodiments, the synthetic peptide further comprises an amino acid residue (X₀) that is N-terminal to position 1 in SEQ ID NO: 4. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the synthetic peptide comprises or consists essentially of the amino acid sequence of any one of SEQ ID NOs: 5-10.

j. Six- to 30-mer or Six- to 50-mer Synthetic Peptides with PXXPXR Motif (SEQ ID NO: 2)

In certain embodiments described herein, the disclosure relates to a synthetic peptide consisting essentially of (i) 6 to 15, 6 to 16, 6 to 17, 6 to 18, 6 to 19, 6 to 20, 6 to 21, 6 to 22, 6 to 23, 6 to 24, 6 to 25, 6 to 26, 6 to 27, 6 to 28, 6 to 29, or 6 to 30 consecutive amino acids, or (ii) 6 to 15, 6 to 16, 6 to 17, 6 to 18, 6 to 19, 6 to 20, 6 to 21, 6 to22, 6 to23, 6 to24, 6 to25, 6 to26, 6 to27, 6 to28, 6 to29, 6 to30, 6 to 31, 6 to 32, 6 to 33, 6 to34, 6 to35, 6 to36, 6 to 37, 6 to 38, 6 to 39, 6 to 40, 6 to 41, 6 to 42, 6 to 43, 6 to 44, 6 to 45, 6 to 46, 6 to 47, 6 to 48, 6 to 49, or 6 to 50 consecutive amino acids, each including an N-terminal amino acid and a C-terminal amino acid, wherein the synthetic peptide comprises the amino acid sequence of SEQ ID NO 2:

(SEQ ID NO: 2)
PX₁X₂PX₃R,

wherein (i) X₁ and X₃ are each independently any amino acid; (ii) X₂ is selected from the group consisting of selected from the group consisting of alanine, arginine, isoleucine, leucine, lysine, phenylalanine, proline, threonine, tyrosine, and valine; and (iii) the synthetic peptide exhibits binding to the SH3-2 domain of NCK1 with a K_{D} of <1,000 μM (e.g., <1,000 μM, <600 μM, or <300 μM), for example, as determined by isothermal titration calorimetry (ITC).

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the synthetic peptide consists essentially of an amino acid sequence of at least seven, at least eight, at least nine, at least ten, at least eleven, at least twelve, at least thirteen, at least fourteen, or at least fifteen consecutive amino acids.

In certain embodiments, the synthetic peptide comprises or consists essentially of the amino acid sequence of any one of SEQ ID NOs: 5-10.

In certain embodiments described herein, the disclosure relates to a synthetic peptide consisting essentially of (i) 6 to 15, 6 to 16, 6 to 17, 6 to 18, 6 to 19, 6 to 20, 6 to 21, 6 to 22, 6 to 23, 6 to 24, 6 to 25, 6 to 26, 6 to 27, 6 to 28, 6 to 29, or 6 to 30 consecutive amino acids, or (ii) 6 to 15, 6 to 16, 6 to 17, 6 to 18, 6 to 19, 6 to 20, 6 to 21, 6 to22, 6 to23, 6 to24, 6 to25, 6 to26, 6 to27, 6 to28, 6 to29, 6 to30, 6 to 31, 6 to 32, 6 to 33, 6 to34, 6 to35, 6 to36, 6 to 37, 6 to 38, 6 to 39, 6 to 40, 6 to 41, 6 to 42, 6 to 43, 6 to 44, 6 to 45, 6 to 46, 6 to 47, 6 to 48, 6 to 49, or 6 to 50 consecutive amino acids, each including an N-terminal amino acid and a C-terminal amino acid, wherein the synthetic peptide comprises the amino acid sequence of SEQ ID NO 2:

(SEQ ID NO: 2)
PX₁X₂PX₃R,

wherein (i) X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine; (ii) X₂ is selected from the group consisting of alanine, arginine, isoleucine, leucine, lysine, phenylalanine, proline, threonine, tyrosine, and valine; and (iii) X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the synthetic peptide consists essentially of an amino acid sequence of at least seven, at least eight, at least nine, at least ten, at least eleven, at least twelve, at least thirteen, at least fourteen, or at least fifteen consecutive amino acids.

In certain embodiments, the synthetic peptide comprises or consists essentially of the amino acid sequence of any one of SEQ ID NOs: 5-10.

In certain embodiments described herein, the disclosure relates to a synthetic peptide consisting essentially of (i) 6 to 15, 6 to 16, 6 to 17, 6 to 18, 6 to 19, 6 to 20, 6 to 21, 6 to 22, 6 to 23, 6 to 24, 6 to 25, 6 to 26, 6 to 27, 6 to 28, 6 to 29, or 6 to 30 consecutive amino acids, or (ii) 6 to 15, 6 to 16, 6 to 17, 6 to 18, 6 to 19, 6 to 20, 6 to 21, 6 to22, 6 to23, 6 to24, 6 to25, 6 to26, 6 to27, 6 to28, 6 to29, 6 to30, 6 to 31, 6 to 32, 6 to 33, 6 to34, 6 to35, 6 to36, 6 to 37, 6 to 38, 6 to39, 6 to40, 6 to41, 6 to42, 6 to 43, 6 to 44, 6 to 45, 6 to46, 6 to47, 6 to48, 6 to49, or 6 to 50 consecutive amino acids, each including an N-terminal amino acid and a C-terminal amino acid, wherein the synthetic peptide comprises the amino acid sequence of SEQ ID NO 2:

(SEQ ID NO: 2)
PX₁X₂PX₃R,

wherein (i) X₁ and X₃ are each independently any amino acid, and (ii) X₂ is a non-aromatic amino acid.

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is an uncharged, non-aromatic amino acid. In certain embodiments, X₂ is alanine or proline. In certain embodiments, X₂ is proline. In certain embodiments, X₂ is a hydrophobic, non-aromatic amino acid. In certain embodiments, X₂ is alanine. In certain embodiments, X₂ is a charged amino acid, for example, a positively charged amino acid. In certain embodiments, X₂ is arginine.

In certain embodiments, X₂ is selected from the group consisting of alanine, arginine, isoleucine, leucine, lysine, proline, threonine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₁ and X₃ are both charged amino acids. In certain embodiments, X₁ and X₃ are both positively charged amino acids. In certain embodiments, X₁ and X₃ are each independently selected from arginine, histidine, and lysine. In certain embodiments, X₁ is arginine and X₃ is lysine.

In certain embodiments, X₁ is a charged amino acid, e.g. a positively charged amino acid, and X₂ is an uncharged amino acid, e.g., a hydrophobic amino acid. In certain embodiments, X₁ is arginine, histidine or lysine, and X₂ is alanine or proline. In certain embodiments, X₁ is arginine and X₂ is proline.

In certain embodiments, X₂ is an uncharged amino acid, e.g., a hydrophobic amino acid, and X₃ is a charged amino acid, e.g., a positively charged amino acid. In certain embodiments, X₂ is alanine or proline, and X₃ is arginine, histidine or lysine. In certain embodiments, X₂ is proline and X₃ is lysine.

In certain embodiments, X₁ is a charged amino acid, e.g., a positively charged amino acid; X₂ is an uncharged amino acid, e.g., a hydrophobic amino acid; and/or X₃ is a charged amino acid, e.g., a positively charged amino acid. In certain embodiments, X₁ is arginine, histidine, or lysine; X₂ is alanine or proline; and/or X₃ is arginine, histidine, or lysine. In certain embodiments, X₁ is arginine, X₂ is proline, and X₃ is lysine.

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the synthetic peptide consists essentially of an amino acid sequence of at least seven, at least eight, at least nine, at least ten, at least eleven, at least twelve, at least thirteen, at least fourteen, or at least fifteen consecutive amino acids.

In certain embodiments, the synthetic peptide comprises or consists essentially of the amino acid sequence of any one of SEQ ID NOs: 5-10.

In certain embodiments described herein, the disclosure relates to a synthetic peptide consisting essentially of (i) 6 to 15, 6 to 16, 6 to 17, 6 to 18, 6 to 19, 6 to 20, 6 to 21, 6 to 22, 6 to 23, 6 to 24, 6 to 25, 6 to 26, 6 to 27, 6 to 28, 6 to 29, or 6 to 30 consecutive amino acids, or (ii) 6 to 15, 6 to 16, 6 to 17, 6 to 18, 6 to 19, 6 to 20, 6 to 21, 6 to22, 6 to23, 6 to24, 6 to25, 6 to26, 6 to27, 6 to28, 6 to29, 6 to30, 6 to 31, 6 to 32, 6 to 33, 6 to34, 6 to35, 6 to36, 6 to 37, 6 to 38, 6 to 39, 6 to 40, 6 to 41, 6 to 42, 6 to 43, 6 to 44, 6 to 45, 6 to 46, 6 to 47, 6 to 48, 6 to 49, or 6 to 50 consecutive amino acids, each including an N-terminal amino acid and a C-terminal amino acid, wherein the synthetic peptide comprises the amino acid sequence of SEQ ID NO 2:

(SEQ ID NO: 2)
PX₁X₂PX₃R,

wherein (i) X₁ and X₃ are each independently any amino acid, and (ii) X₂ is selected from the group consisting of alanine, arginine, isoleucine, leucine, lysine, phenylalanine, proline, threonine, tyrosine, and valine.

In certain embodiments, X₁ is a charged amino acid, for example, a positively charged amino acid or a negatively charged amino acid. In certain embodiments, X₁ is arginine, histidine, or lysine. In certain embodiments, X₁ is arginine. In certain embodiments, X₁ is glutamic acid. In certain embodiments, X₁ is an uncharged amino acid. In certain embodiments, X₁ is a hydrophobic amino acid. In certain embodiments, X₁ is alanine, asparagine, or serine.

In certain embodiments, X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine. In certain embodiments, X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

In certain embodiments, X₂ is selected from the group consisting of alanine, arginine, isoleucine, leucine, lysine, proline, threonine, tyrosine, and valine. In certain embodiments, X₂ is selected from the group consisting of alanine, isoleucine, proline, and valine. In certain embodiments, X₂ is selected from the group consisting of isoleucine, proline, and valine.

In certain embodiments, X₃ is a charged amino acid, for example a positively charged amino acid or a negatively charged amino acid. In certain embodiments, X₃ is arginine, histidine, or lysine. In certain embodiments, X₃ is lysine. In certain embodiments, X₃ is glutamic acid. In certain embodiments, X₃ is an uncharged amino acid. In certain embodiments, X₃ is alanine, asparagine, cysteine, serine, or tryptophan. In certain embodiments, X₃ is a hydrophobic amino acid. In certain embodiments, X₃ is alanine, cysteine, or tryptophan.

In certain embodiments, X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. In certain embodiments, X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, the synthetic peptide further comprises an amino acid residue (X₀) that is N-terminal to position 1 in SEQ ID NO: 2. In certain embodiments, X₀ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

In certain embodiments, the synthetic peptide consists essentially of an amino acid sequence of at least seven, at least eight, at least nine, at least ten, at least eleven, at least twelve, at least thirteen, at least fourteen, or at least fifteen consecutive amino acids.

In certain embodiments, the synthetic peptide comprises or consists essentially of the amino acid sequence of any one of SEQ ID NOs: 5-10.

### III. Pharmaceutical Compositions

For therapeutic use, a synthetic NCK1-binding peptide of the disclosure preferably is combined with a pharmaceutically acceptable carrier.

Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. Suitable formulations for use in the present disclosure are found in Adeboye Adejare, Remington: The Science and Practice of Pharmacy (23d ed. 2020). For a brief review of methods for drug delivery, see, e.g., Langer, Science (1990) 249:1527-1533, 1990.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); to nicity enhancing agents (such as alkali metal halides, preferably sodium or potassium chloride, mannitol sorbitol); delivery vehicles; diluents; excipients and/or pharmaceutical adjuvants (see Adeboye Adejare, Remington: The Science and Practice of Pharmacy (23d ed. 2020)).

The disclosure provides a pharmaceutical composition comprising a synthetic peptide consisting essentially of an amino acid sequence of (i) six to thirty or (ii) six to fifty, consecutive amino acids including an N-terminal amino acid and a C-terminal amino acid, wherein the amino acid sequence comprises the amino acid sequence of PX₁X₂PX₃R (SEQ ID NO: 2), wherein X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine; X₂ is selected from the group consisting of alanine, arginine, isoleucine, leucine, lysine, phenylalanine, proline, threonine, tyrosine, and valine; and X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In certain embodiments, X₁ is selected from (i) the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine or (ii) alanine, arginine, asparagine, aspartic acid, cysteine, glutamic acid, glutamine, histidine, isoleucine, leucine, lysine, methionine, phenylalanine, proline, serine, threonine, tyrosine, and valine. Alternatively or in addition to each of the foregoing, X₂ is selected from (i) the group consisting of alanine, arginine, isoleucine, leucine, lysine, proline, threonine, tyrosine, and valine, (ii) the group consisting of alanine, isoleucine, proline, and valine or (iii) the group consisting of isoleucine, proline, and valine. Alternatively or in addition to each of the foregoing, X₃ is selected from (i) the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine or (ii) the group consisting of alanine, arginine, asparagine, cysteine, glutamic acid, glutamine, histidine, isoleucine, leucine, lysine, methionine, proline, serine, threonine, tryptophan, tyrosine, and valine.

In certain embodiments, a synthetic NCK1-binding peptide of the disclosure is formulated as a salt, e.g., as an acetate salt. In certain embodiments, the acetate salt a bis-acetate salt or as a tri-acetate salt. In certain embodiments, the peptide is formulated as an acetate salt and combined with a pharmaceutically acceptable carrier. In certain embodiments, at least one amino acid or at least two amino acid residues are covalently linked to a chemical moiety not present in a natural amino acid. In certain embodiments, the disclosure provides a composition (e.g., a pharmaceutical composition) comprising a synthetic NCK1-binding peptide formulated as a free base or as a salt, as described herein.

In certain embodiments, a composition (e.g., a pharmaceutical composition) may comprise a synthetic NCK1-binding peptide of the disclosure, wherein the purity of the synthetic peptide is at least 95%. For example, the purify of the synthetic peptide in the composition may be at least 95%, at least 96%, at least 97%, at least 98%, at least 99%, or at least 99.5%.

Pharmaceutical compositions containing a synthetic NCK1-binding peptide as disclosed herein can optionally be dried, using any appropriate method known to those skilled in the art. The pharmaceutical composition can, for example, be freeze dried or lyophilized.

In certain embodiments, a pharmaceutical composition may contain nanoparticles, or lipid droplets, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid.

Sustained release compositions may also include liposomes that can be prepared by any of several methods known in the art.

Pharmaceutical compositions containing a synthetic NCK1-binding peptide as disclosed herein can be presented in a dosage unit form and can be prepared by any suitable method. A pharmaceutical composition should be formulated to be compatible with its intended route of administration. Examples of routes of administration are subcutaneous (SC), intravenous (IV), intraperitoneal, intradermal, inhalation, transdermal, to pical, transmucosal, intrathecal, and rectal administration. In certain embodiments, the synthetic peptide is administered by subcutaneous administration.

Useful formulations can be prepared by methods known in the pharmaceutical art. For example, see Adeboye Adejare, Remington: The Science and Practice of Pharmacy (23d ed. 2020). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of to nicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes, gamma radiation, etc. Where the composition is lyophilized, filter sterilization can be conducted prior to or following lyophilization and reconstitution.

The compositions described herein may be administered locally or systemically. It is contemplated that the compositions described herein are generally administered by parenteral administration. Preparations for parenteral administration include sterile aqueous or non-aqueous solutions, suspensions, and emulsions. In certain embodiments, the pharmaceutical composition is administered subcutaneously or may be administered intravenously, e.g., via intravenous infusion. In certain embodiments, it is contemplated that the synthetic constructs disclosed herein can be administered by systemic administration.

In embodiments wherein the compositions (e.g., pharmaceutical compositions) of the disclosure are aqueous, the composition can comprise a synthetic peptide of the disclosure at a concentration of, e.g., 150 mg/mL to 400 mg/mL. For example, the concentration of the synthetic peptide in the aqueous composition can be 150 mg/mL to 400 mg/mL, 150 mg/mL to 300 mg/mL, 150 mg/mL to 200 mg/mL, 200 mg/mL to 400 mg/mL, 200 mg/mL to 300 mg/mL, or 300 mg/mL to 400 mg/mL. In certain embodiments, the concentration of the synthetic peptide in the aqueous composition is 150 mg/mL, 200 mg/mL, 300 mg/mL, or 400 mg/mL. In certain embodiments, the aqueous composition is injectable (e.g., suitable for subcutaneous injection).

Generally, a therapeutically effective amount of active component, for example, a synthetic NCK1-binding peptide of the disclosure, is in the range of 0.1 mg/kg to 100 mg/kg, e.g., 1 mg/kg to 100 mg/kg. In certain embodiments, the effective amount is in the range of 15 to 30 mg/kg. In certain embodiments, the effective amount is 15 mg/kg. In certain embodiments, the effective amount is 30 mg/kg. In certain embodiments, the effective amount is in the range of 30 mg/kg to 150 mg/kg, e.g., 30 mg/kg to 75 mg/kg or 75 mg/kg to 150 mg/kg.

In certain embodiments, the effective amount is 30 mg/kg. In certain embodiments, the effective amount is 75 mg/kg. In certain embodiments, the effective amount is 150 mg/kg. In certain embodiments, the effective amount is in the range of 50 mg/kg to 350 mg/kg, e.g., 50 mg/kg to 175 mg/kg or 175 mg/kg to 350 mg/kg. In certain embodiments, the effective amount is 50 mg/kg. In certain embodiments, the effective amount is 175 mg/kg. In certain embodiments, the effective amount is 350 mg/kg. In certain embodiments, the effective amount of a synthetic NCK1-binding peptide of the disclosure (e.g., formulated as an acetate salt) is in the range of 40 mg to 320 mg. For example, in certain embodiments, the effective amount is in the range of 40 mg to 320 mg, 40 mg to 240 mg, 40 mg to 160 mg, 40 mg to 80 mg, 80 mg to 320 mg, 80 mg to 240 mg, 80 mg to 160 mg, 160 mg to 320 mg, 160 mg to 240 mg, or 240 mg to 320 mg. In certain embodiments, the effective amount is 40 mg, 80 mg, 160 mg, 240 mg, or 320 mg. In certain embodiments, the effective amount of a synthetic NCK1-binding peptide of the disclosure (e.g., formulated or measured as a free base) is in the range of 30 mg to 270 mg. For example, in certain embodiments, the effective amount is in the range of 30 mg to 270 mg, 30 mg to 200 mg, 30 mg to 140 mg, 30 mg to 70 mg, 60 mg to 270 mg, 60 mg to 200 mg, 60 mg to 140 mg, 130 mg to 270 mg, 130 mg to 200 mg, or 190 mg to 270 mg. In certain embodiments, the effective amount is 30 mg, 60 mg, 70 mg, 130 mg, 140 mg, 190 mg, 200 mg, or 270 mg.

The amount administered will depend on variables such as the type and extent of disease or indication to be treated, the overall health of the patient, the in vivo potency of the active component, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study. Dosing frequency can vary, depending on factors such as route of administration, dosage amount, serum half-life of the synthetic peptide, and the disease, disorder, or condition being treated. Exemplary dosing frequencies are once per day, once per week and once every two weeks.

The disclosure also provides a delivery system comprising (1) a composition (e.g., a pharmaceutical composition) disclosed herein, and (2) a delivery device. The composition can be, for example, a composition (e.g., a pharmaceutical composition) comprising a synthetic NCK1-binding peptide of the disclosure, optionally wherein the peptide is formulated as a salt, e.g., an acetate salt, e.g., a bis-acetate salt or a tri-acetate salt. In certain embodiments, the delivery device is suitable for administering the composition comprising a synthetic peptide of the disclosure to a subject in need thereof, e.g., via subcutaneous or intravenous injection. In certain embodiments, the delivery device is a syringe, a pen delivery device, an injector delivery system, and/or an autoinjector. In certain embodiments, the delivery device is a syringe, e.g., a pre-filled syringe.

### IV. Methods and Therapeutic Uses

The disclosure also provides a method of modulating an activity of NCK1, wherein the method comprises contacting NCK1 with an agent that disrupts an interaction between PTP1B and NCK1, thereby modulating or inhibiting binding between NCK1 and PTP1B. In certain embodiments, the agent is a small molecule, protein, e.g., an antibody (e.g., an antibody or antigen binding fragment thereof that binds PTP1B or NCK1 to reduce or prevent the interaction between PTP1B and NCK1) or peptide, e.g., a synthetic peptide or composition of the disclosure that can reduce or prevent the interaction between PTP1B and NCK1. In certain embodiments, contacting NCK1 with the agent disrupts binding between PTP1B and NCK1. Binding of PTP1B and NCK1 can be measured using any number of techniques known in the art including, for example, via co-immunoprecipitation assays, via isothermal Titration Calorimetry (ITC), or via Surface Plasmon Resonance (SPR). In certain embodiments, contacting NCK1 results in modulating an activity of PTP1B. In certain embodiments, the activity of PTP1B is downregulation of the insulin receptor signaling pathway. In certain embodiments, the activity of PTP1B is dephosphorylation of the insulin receptor.

The disclosure also provides a method of increasing insulin receptor signaling, e.g., in a subject, cell, or tissue, wherein the method comprises contacting NCK1 with an agent, e.g., a protein, e.g., an antibody (e.g., an antibody or antigen binding fragment thereof that binds PTP1B or NCK1 to reduce or prevent the interaction between PTP1B and NCK1) or a peptide, e.g., a synthetic peptide or composition of the disclosure that can reduce or prevent the interaction between PTP1B and NCK1, thereby increasing insulin receptor signaling.

The present disclosure provides methods for decreasing or inhibiting binding between PTP1B and NCK1, by contacting NCK1 with an effective amount of an agent that disrupts an interaction between PTP1B and NCK1, thereby modulating or inhibiting binding between NCK1 and PTP1B. In certain embodiments, the agent is a small molecule, protein (e.g., an antibody or antigen fragment thereof that binds to PTP1B or NCK1), or a peptide (e.g., a synthetic NCK1-binding peptide as disclosed herein), each of which can disrupt or otherwise interfere with the formation of a PTP1B-NCK1 complex. In certain embodiments, the method is used to modulate an activity of PTP1B, e.g., PTP1B-mediated downregulation of insulin receptor signaling, thereby enhancing or restoring sensitivity to insulin and/or insulin receptor signaling in a cell or tissue. Without wishing to be bound by theory, it is believed that by using agents (e.g., peptides) that target and bind NCK1 (rather than PTP1B), that administration of the agent (e.g. peptide) will preferentially or specifically inhibit PTP1B activity that is dependent on interaction with the NCK1 adapter protein (e.g., downregulation of insulin receptor signaling), while having less or a minimal impact on, e.g., PTP1B-mediated regulation of other targets or pathways.

Each of the compositions and methods disclosed herein can be used to treat or prevent a disease or disorder associated with PTP1B and/or NCK1. In certain embodiments, the disclosure provides administering an agent (e.g., a peptide) that disrupts an interaction between PTP1B and NCK1 to a subject in need thereof. In certain embodiments, the agent is a NCK1-binding peptide of the disclosure. In certain embodiments, the agent is a NCK1-binding peptide as described in, e.g., any one of sections II(a) through II(j) of this disclosure. In certain embodiments, the agent is a NCK1-binding peptide as described in section II(j) of this disclosure. In certain embodiments, the agent is a NCK1-binding peptide as set forth in Table 1 or Table 2. In certain embodiments, the agent is a NCK1-binding peptide as set forth in Table 2.

The disclosure provides a method of treating or preventing a disease or disorder associated with PTP1B and/or NCK1. In certain embodiments, the method comprises administering a NCK1-binding peptide of the disclosure to a subject in need thereof. The method can comprise administering to the subject an effective amount of a synthetic NCK1-binding peptide described herein, either alone or in a combination with another therapeutic agent to treat or prevent the disease or disorder in the subject. An effective amount of the peptide can be administered in one or more administrations, applications, or dosages and is not intended to be limited to a particular formulation or administration route.

In certain embodiments, the disease or disorder is a disease or disorder associated with NCK1 activity and/or PTP1B activity. In certain embodiments, the disease or disorder is a metabolic disease or disorder. In certain embodiments, the diseases or disorder is diabetes, obesity, insulin resistance, hyperlipidemia, or atherosclerosis. In certain embodiments, the disease or disorder is diabetes, e.g., Type 2 diabetes. In certain embodiments, the disease or disorder is obesity.

In one embodiment, the disclosure provides a method of treating or preventing diabetes (e.g., Type 2 diabetes) in a subject in need thereof, the method comprising administering to the subject an effective amount of a synthetic peptide described herein or a pharmaceutical composition comprising the synthetic peptide and a pharmaceutically acceptable carrier, thereby to treat or prevent diabetes in the subject, wherein the synthetic peptide consists essentially of an amino acid sequence of (i) six to thirty or (ii) six to fifty consecutive amino acids including an N-terminal amino acid and a C-terminal amino acid, wherein the amino acid sequence comprises the amino acid sequence of PX₁X₂PX₃R (SEQ ID NO: 2), wherein (1) X₁ and X₃ are each independently any amino acid; and (2) X₂ is a non-aromatic amino acid. In some embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In another embodiment, the disclosure provides a method of treating or preventing diabetes (e.g., Type 2 diabetes) in a subject in need thereof, the method comprising administering to the subject an effective amount of a synthetic peptide or a pharmaceutical composition comprising the synthetic peptide and a pharmaceutically acceptable carrier, thereby to treat or prevent diabetes in the subject, wherein the synthetic peptide consists essentially of an amino acid sequence of (i) six to thirty or (ii) six to fifty consecutive amino acids including an N-terminal amino acid and a C-terminal amino acid, wherein the amino acid sequence comprises the amino acid sequence of PX₁X₂PX₃R (SEQ ID NO: 2), wherein (1) X₁ and X₃ are each independently any amino acid; and (2) X₂ is selected from the group consisting of alanine, arginine, isoleucine, leucine, lysine, phenylalanine, proline, threonine, tyrosine, and valine. In some embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In addition, the disclosure provides a method of treating obesity in a subject in need thereof, the method comprising administering to the subject an effective amount of a synthetic peptide or a pharmaceutical composition comprising the synthetic peptide and a pharmaceutically acceptable carrier, thereby to treat obesity in the subject, wherein the synthetic peptide consists essentially of an amino acid sequence of (i) six to thirty or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is a non-aromatic amino acid. In some embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In another embodiment, the disclosure provides a method of treating obesity in a subject in need thereof, the method comprising administering to the subject an effective amount of a synthetic peptide or a pharmaceutical composition comprising the synthetic peptide and a pharmaceutically acceptable carrier, thereby to treat obesity in the subject, wherein the synthetic peptide consists essentially of an amino acid sequence of (i) six to thirty or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is selected from the group consisting of alanine, arginine, isoleucine, leucine, lysine, phenylalanine, proline, threonine, tyrosine, and valine. In some embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., insulin therapy, surgery, or administration of another therapeutic preparation.

The disclosure also provides a method of decreasing fat body fat mass in a subject in need thereof, wherein the method comprises administering to the subject an effective amount of a synthetic peptide of the disclosure or pharmaceutical composition comprising the same. In certain embodiments, body fat mass in the subject is decreased by at least about 5%, at least about 10%, at least about 15%, at least about 20%, at least about 30%, at least about 40%, at least about 50%, at least about 60%, at least about 70%, at least about 80%, or at least about 90%. The disclosure also provides a method of reducing or lessening an increase in body fat mass in a subject, e.g., as compared to an appropriate standard or control, wherein the method comprises administering to the subject an effective amount of a synthetic peptide of the disclosure or pharmaceutical composition comprising the same. In certain embodiments, the appropriate standard or control is determined according to the rate of body fat mass gain in the same subject prior to administration of the synthetic peptide or the pharmaceutical composition.

In certain embodiments, the appropriate standard or control is a subject or population who is not administered the peptide or composition of the disclosure. In certain embodiments, administration of the synthetic peptide or composition does not result in a substantial change in lean body mass in the subject. In certain embodiments, administration of the synthetic peptide or composition does not result in a decrease in lean body mass in the subject.

The disclosure also provides a method of decreasing body weight in a subject in need thereof, wherein the method comprises administering to the subject an effective amount of a synthetic peptide of the disclosure or pharmaceutical composition comprising the same. In certain embodiments, body weight of the subject is decreased by at least about 1%, at least about 2%, at least about 3%, at least about 4%, at least about 5%, at least about 6%, at least about 8%, at least about 10%, at least about 12%, at least about 15%, at least about 20%, at least about 25%, at least about 30%, at least about 40%, or at least about 50%. The disclosure also provides a method of reducing or lessening an increase in body weight of a subject, e.g., as compared to an appropriate standard or control, wherein the method comprises administering to the subject an effective amount of a synthetic peptide of the disclosure or pharmaceutical composition comprising the same. In certain embodiments, the appropriate standard or control is determined according to the rate of body weight gain in the same subject prior to administration of the synthetic peptide or the pharmaceutical composition. In certain embodiments, the appropriate standard or control is a subject or population who is not administered the peptide or composition of the disclosure. In certain embodiments, administration of the synthetic peptide or composition does not result in a substantial change in lean body mass in the subject. In certain embodiments, administration of the synthetic peptide or composition does not result in a decrease in lean body mass in the subject.

In one embodiment, the disclosure provides a method of decreasing body fat mass in a subject in need thereof, the method comprising administering to the subject an effective amount of a synthetic peptide described herein or a pharmaceutical composition comprising a synthetic peptide disclosed herein and a pharmaceutically acceptable carrier, thereby to decrease body fat mass of the subject, wherein the synthetic peptide consists essentially of an amino acid sequence of (i) six to thirty or (ii) or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is a non-aromatic amino acid. In some embodiments, if the proline at position 1 of SEQ ID NO: 2 is the N-terminal amino acid of the synthetic peptide, then the N-terminal amino acid is modified with a chemical moiety (e.g., by covalent linkage to a carbonyl-containing moiety).

In another embodiment, the disclosure provides a method of decreasing body fat mass in a subject in need thereof, the method comprising administering to the subject an effective amount of a synthetic peptide described herein or a pharmaceutical composition comprising a synthetic peptide disclosed herein and a pharmaceutically acceptable carrier, thereby to decrease body fat mass of the subject, wherein the synthetic peptide consists essentially of an amino acid sequence of (i) six to thirty or (ii) six to fifty, consecutive amino acids, including an N-terminal amino acid and a C-terminal amino acid, wherein the amino acid sequence comprises the amino acid sequence of PX₁X₂PX₃R (SEQ ID NO: 2), wherein: (1) X₁ and X₃ are each independently any amino acid; and (2) X₂ is selected from the group consisting of alanine, arginine, isoleucine, leucine, lysine, phenylalanine, proline, threonine, tyrosine, and valine. In some embodiments, if the proline at position 1 of SEQ ID NO: 2 is the N-terminal amino acid of the synthetic peptide, then the N-terminal amino acid is modified with a chemical moiety (e.g., covalent linkage to a carbonyl-containing moiety).

### V. Methods of Making Synthetic Peptides

Methods of making a synthetic NCK1-binding peptide as described herein are known in the art. For example, DNA molecules encoding a synthetic peptide can be synthesized chemically or by recombinant DNA methodologies. The sequences of interest can be cloned by conventional hybridization techniques or polymerase chain reaction (PCR) techniques, using the appropriate synthetic nucleic acid primers. The resulting DNA molecules encoding the synthetic peptide can be ligated to other appropriate nucleotide sequences, including, for example, expression control sequences, to produce conventional gene expression constructs (i.e., expression vectors) encoding the desired peptides. Production of defined gene constructs is within routine skill in the art.

Nucleic acids encoding a synthetic peptide can be incorporated (ligated) into expression vectors, which can be introduced in to host cells through conventional transfection or transformation techniques. Exemplary host cells are E. coli cells, S. cerevisiae cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the synthetic peptide.

Specific expression and purification conditions will vary depending upon the expression system employed. For example, if a gene is to be expressed in E. coli, it is first cloned in to an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., Saccharomyces cerevisiae cells or CHO cells, it is first inserted in to an expression vector containing a suitable eukaryotic promoter, a secretion signal, a poly A sequence, and a stop codon. Optionally, the vector or gene construct may contain enhancers and introns. The gene construct can be introduced in to eukaryotic host cells using conventional techniques.

A peptide as described herein can be produced by growing (culturing) a host cell transfected with an expression vector encoding such a variable region, under conditions that permit expression of the polypeptide. Following expression, the peptide can be harvested and purified or isolated using techniques known in the art, e.g., affinity tags such as glutathione-S-transferase (GST) or histidine tags. In certain embodiments, an N-terminal signal sequence is included in the protein construct. Exemplary N-terminal signal sequences include signal sequences from interleukin-2, CD-5, IgG kappa light chain, trypsinogen, serum albumin, and prolactin.

The peptides can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

In certain embodiments, peptides of the disclosure may be chemically synthesized. Methods of chemically synthesizing polypeptides are well known in the art and include, but are not limited to, solid-phase peptide synthesis, liquid-phase peptide synthesis, and organic synthesis methods. In some peptide synthesis methods, an amino group of one amino acid (or amino acid derivative) is linked to a carboxyl group of another amino acid (or amino acid derivative) that has been activated by reacting it with a reagent such as dicyclohexylcarbodiimide (DCC). When the free amino group attacks the activated carboxyl group, a peptide bond is formed and dicyclohexylurea is released. In such methods, other potentially reactive groups (such as the a-amino group of the N-terminal amino acid or amino acid derivative and the carboxyl group of the C-terminal amino acid or amino acid derivative) may be blocked (“protected”) from participating in the chemical reaction. Thus, only particular active groups react such that the desired product is formed. Blocking groups useful for this purpose include, without limitation, tertbutoxycarbonyl groups (t-Boc) and benzoyloxycarbonyl groups to protect amine groups; and simple esters (such as methyl and ethyl groups) and benzyl esters to protect carboxyl groups. Blocking groups can typically be subsequently removed with a treatment that leaves peptide bonds intact (for example, treatment with dilute acid). This process of protecting reacting groups that should not react, coupling to form a peptide bond, and deprotecting reactive groups may be repeated. A peptide may be synthesized by sequentially adding amino acids to a growing peptide chain. Both liquid-phase and solid phase peptide synthesis methods are suitable for use in accordance with the invention. In solid-phase peptide synthesis methods, the growing peptide chain is typically linked to an insoluble matrix (such as, for example, polystyrene beads) by linking the carboxyterminal amino acid to the matrix. At the end of synthesis, the peptide can be released from the matrix using a cleaving reagent that does not disrupt peptide bonds, such as hydrofluoric acid (HF). Protecting groups are also typically removed at this time. Automated, high throughput, and/or parallel peptide synthesis methods may also be used in accordance with the invention. For more information about peptide synthesis methods, see, e.g., Merrifield (1969) ADV. ENZYMOL. RELAT. AREAS MOL. BIOL., 32: 221-96; Fridkin et al. (1974) ANNU. REV. BIOCHEM. 43(0): 419-43; Merrifield (1997) METH. ENZYMOL. 289: 3-13; Sabatino et al. (2009) CURR. OPIN. DRUG DISCOV. DEVEL., 11(6): 762-70, the entire contents of each of which are herein incorporated by reference.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps. Similarly, throughout the description, where compositions are described as consisting essentially of specific components, or where processes and methods are described as consisting essentially of specific steps, it is contemplated that, additionally, there are compositions of the present invention that consist of the recited components, and that there are processes and methods according to the present invention that consist of the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

As used herein, the terms “SEQ ID NO: 1,” “SEQ ID NO: 2,” “SEQ ID NO: 3,” and “SEQ ID NO: 4” should be understood to have the same meaning as “Formula I,” “Formula II,” “Formula III,” and “Formula IV,” respectively, unless otherwise specifically stated or understood from the context.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1. Binding Interaction Between PTP1B and NCK1

This example demonstrates the interaction between PTP1B and NCK1 and where within NCK1 this interaction takes place using in vitro immunoprecipitation experiments performed with tagged PTP1B and NCK1 proteins.

### Materials and Methods

Plasmid Construction: Human PTP1B cDNA (NM_002827) and human NCK1 cDNA (NM_006153) were obtained (Origene, MD, USA). FLAG-tagged PTP1B (FLAG-PTP1B) and HA-tagged NCK1 (HA-NCK1) were amplified from the PTP1B and NCK1 cDNA, respectively.

Amplicons were double digested (EcoRI and XhoI, New England Biolabs), ligated into pcDNA3.1 vector (#M2200S, New England Biolabs; vector from Invitrogen) and transformed in to DH5α E. coli cells (#C2987H, NEB). Constructs were purified and eluted using a plasmid preparation kit according to manufacturer's instructions (#T1010, New England Biolabs).

Cell culture and co-transfection: Human embryonic kidney 293 (HEK293) cells were grown in Dulbecco's modified Eagle's medium (DMEM, Gibco) containing 10% fetal bovine serum (FBS), at 37 °C in a 5% CO₂ environment. Plasmids encoding FLAG-PTP1B and HA-NCK1 were transiently expressed in HEK293 cells using Lipofectamine 2000 (Invitrogen), and DMEM supplemented with 200 μg/mL G418 was used to grow transfected cells.

Cell lysates, immunoblotting and co-immunoprecipitation: Cells were washed with PBS and soluble proteins were extracted using immunoprecipitation (IP) buffer (0.1% Triton×100, 150 mM NaCl, 25 mM KCl, 10 Mm Tri HCl, 1 mM CaCl₂) in dH₂O (pH 7.4)) with protease inhibitors. Cells were centrifuged at 12,500 g for 10 min at 4 °C and supernatants were collected. For FLAG-PTP1B and HA-NCK1 coimmunoprecipitation, cell lysates were incubated overnight at 4 °C with anti-FLAG HRP antibody (#A8592, Sigma) or anti-HA (#MMS-101P, BioLegend) antibodies and glutathione-agarose beads (#G4510, Sigma). Immunoprecipitated proteins were washed three times with IP buffer and eluted in SDS-Laemmli buffer by heating at 95 °C for 10 min. Samples were spun at 12,000 rpm for 2 min, and the liquid fraction was transferred in to 1 mL Eppendorf tubes. Chemiluminescent Western blotting was used to separate and visualize proteins (PEQLAB, Germany).

Construction of GST-fusion constructs: Full-length NCK1 fused to GST and NCK1 SH3-2 domain fused to GST were amplified from NCK1 cDNA (Addgene). Amplicons were double digested (EcoRI and XhoI, New England Biolabs), ligated in to pGEX-6P-2 vector (#M2200S, New England Biolabs; #GE28-9546-50, Merck) and transformed in to DH5α E. coli cells (#C2987H, New England Biolabs). Constructs were purified using a plasmid preparation kit according to manufacturer's instructions (#T1010, New England Biolabs).

Purification of GST-fusion proteins: GST-constructs were transformed in to BL21-Gold E. coli cells (#230134, Agilent), and protein synthesis was induced using isopropyl-beta-D-thiogalactopyranoside (IPTG) stimulation (#I6758, Merck). GST-fusion proteins were extracted by sonication in STE buffer (10 mM Tris-HCl, 150 mM NaCl, 1 mM EDTA in dH₂O, pH 8) supplemented with protease inhibitor tablets for bacteria, DTT and lysozyme. Samples were spun at 8,000 rpm for 25 min, and lysates were incubated with glutathione-agarose beads overnight (#G4510, Sigma). The following day, beads were washed three times with STE buffer and stored at 4 °C.

### Construction, Synthesis and Purification of His-Tagged PTP1B and His-Tagged NCK1 SH3-2 Domain:

Human PTP1B cDNA (NM_002827) and human NCK1 cDNA (NM 006153) were obtained (Origene, MD, USA). PTP1B Primers were designed to amplify the ORFs with added homology to the cut vector (EcoRI-digested pSF-OXB20-NH2-10HIS) in the forward primer sequence. To amplify full-length PTP1B, the primers ptp1b_FL_fwd (acctgtacttccagggcatggagatggaaaaggagttc) (SEQ ID NO: 159) and ptp1b_FL_rev (gtgcaggaggagttcgaattctatgtgttgctgttgaac) (SEQ ID NO: 160) were used. To amplify truncations of PTP1B, the following primer pairs were used: ptp1b_FL-Fwd and one of Ptplb_323stop_rev (gtgcaggaggagttcgaattTCATTTCCCATTGTGTGG) (SEQ ID NO: 161), ptp1b_344stop_rev (gtgcaggaggagttcgaatttcagtctttatcctcctg) (SEQ ID NO: 162) or ptp1b_278stop_rev(gtgcaggaggagttcgaattttaggcaccttcgatcac) (SEQ ID NO: 163). To amplify the NCK1 SH3-2 domain, the primers ncklsh32_fwd (acctgtacttccagggcatgatgcccgcttatgtgaaatttaac) (SEQ ID NO: 164) and ncklsh32_rev (gtgcaggaggagttcgaattctagtcaccttcttcagttacatag) (SEQ ID NO: 165) were used. The amplified fragments were purified using a DNA clean-up kit according to manufacturer's instructions (QIAquick PCR purification kit, Qiagen).

The plasmid pSF-OXB20-NH2-10HIS (Sigma Aldrich) was linearized using EcoRI (New England Biolabs). Each amplified fragment was ligated in to the expression vector using NEBuilder® HiFi DNA (New England Biolabs) assembly kit to create the expression constructs, according to the manufacturer's instructions. The reaction was transformed in to competent E. coli (NEB-5a, New England Biolabs) and the presence of the gene fragments was confirmed via colony PCR amplification using GoTaq Hotstart polymerase (Promega) using the primers OGPF2 (TGTCGATCCTACCATCCA) (SEQ ID NO: 166) (45 bp from start codon) and OGPR2 (AGTCAGTCAGTGCAGGAG) (SEQ ID NO: 167) (65 bp from 3′). Constructs containing the target protein coding sequences were transformed in to E. coli strain C43(DE3) (Sigma Aldrich). The transformed E. coli cells were spread on L-agar plates containing 50 g/ml kanamycin. The plates were incubated overnight at 37 °C and colony formation was visually assessed.

For expression solubility screening, an overnight culture was prepared by growing a single transformed E. coli colony in 10 mL of autoclaved lysogeny broth (LB) medium containing 50 g/mL of kanamycin overnight at 37 °C. Small scale (50 mL) expression trials were carried out in a 250 mL baffled flask. Cell cultures were grown at 37 °C in an incubator with a shaking speed of 180 rpm 37 °C for 4 hours, and expression was permitted to continue overnight at 20 °C or 25 °C. Following overnight incubation, cells were collected by centrifugation, resuspended in 5 mL of lysis buffer (10 mM Tris, pH 7.4 or 10 mM Sodium Phosphate, pH 8.0, 300 mM NaCl, 1 mM DTT or TCEP, 0-10% glycerol) and sonicated three times for 10 s over ice with 1 min interval using a small probe at 15 amplitude microns (Soniprep 150, MSE). The soluble fraction was collected by centrifugation for 10-60 min at 20,000 rpm in a microfuge. Constructs expressing soluble protein were identified by SDS-PAGE.

To produce and purify fusion proteins, a culture was inoculated with 1% of overnight-cultured inoculum. Protein expression was carried out under the conditions described above, scaled up as necessary. Cells expressing the recombinant proteins were harvested in 50 mL tubes by centrifugation and stored at −80 °C. Cells containing the expressed gene product were thawed over ice and resuspended in Extraction buffer (10 mM Tris, pH 7.4 or 10 mM Sodium Phosphate, pH 8.0; 300 mM NaCl, 1 mM DTT or TCEP; 0-10% glycerol and 1 μg lysozyme) and sonicated over ice (4 °C) for 3 min total using pulses of 5 s with 15 s intervals, using a wide probe at 15 amplitude microns (Soniprep 150, MSE). Insoluble material was removed from the lysate by centrifugation at 20K rpm for 20-60 min at 4 °C. Supernatant was filtered through a 0.22 μm PVDF filter membrane and applied to 1-2 5 mL HisTrap FF Crude (Cytiva) prepacked Ni columns and connected to an AKTA explorer. After thorough washing with binding buffer (10 mM Tris, pH 7.4 or 10 mM Sodium Phosphate, pH 8.0; 300 mM NaCl; 1 mM DTT or TCEP and 25 mM Imidazole), protein was eluted using elution buffer (10 mM Tris, pH 7.4 or 10 mM Sodium Phosphate, pH 8.0; 150-300 mM NaCl; 1 mM DTT or TCEP and 250 mM Imidazole). Fractions containing protein of interest were confirmed by SDS PAGE. Protein was diluted with GF buffer (10 mM Tris, pH 7.4 or 10 mM Sodium Phosphate, pH 8.0; 150-300 mM NaCl) and subjected to TEV protease treatment overnight at 4-10 °C. Following proteolytic digestion, HIS-tag and TEV protease were removed by filtration through a clean 5 mL HisTrap FF Crude (Cytiva) prepacked Ni column. Protein was concentrated on a PES ultrafiltration device by centrifugation at 4000 rpm at 4 °C, and protein was polished via a HiPrep S-100 26/60 (Cytiva) using GF buffer (as described above). Purified proteins were concentrated using ultrafiltration, and protein concentration was determined using ultraviolet-visible spectroscopy. The integrity and identity of all proteins were verified by mass spectrographic analysis.

GST pull-down experiments: GST-bound agarose beads of SH3-2 domain and full length NCK1 were incubated with different concentrations of His-tagged PTP1B protein overnight. Beads were three times washed in STE buffer in the morning and proteins eluted in SDS-Laemmli buffer by heating at 95 °C for 10 min. Samples were spun at 12,000 rpm for 2 min, and the liquid fraction was transferred in to 1 mL Eppendorf tubes. Chemiluminescent Western blotting was used to separate and visualize proteins (PEQLAB, Germany).

### Results and Discussion

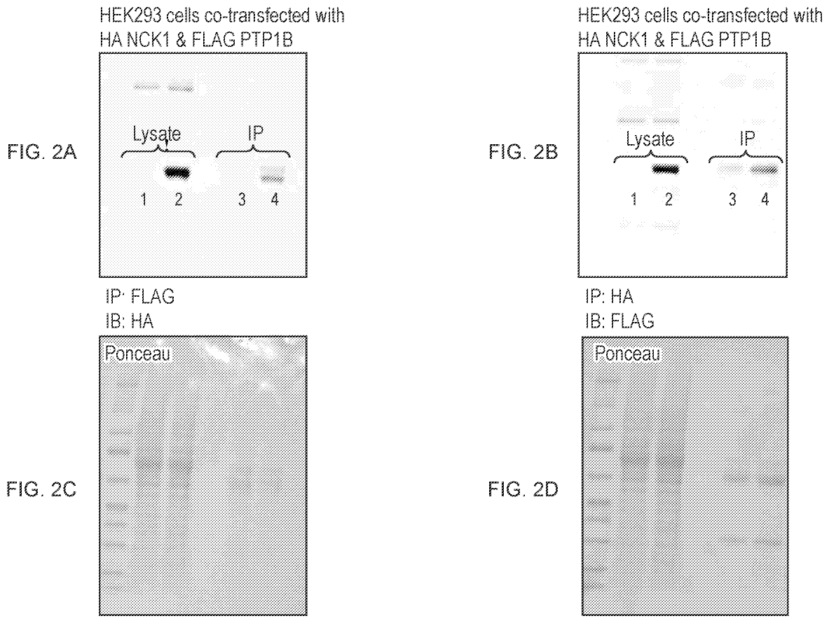

FLAG-PTP1B and HA-NCK1 fusion constructs were generated, and HEK293 cells were co-transfected with both constructs. Non-transfected HEK293 cells were used as negative controls (FIGS. 2A-2B, lanes 1 and 3). Either FLAG-PTP1B (FIG. 2A) or HA-NCK1 (FIG. 2B) was immunoprecipitated from co-transfected cells with anti-FLAG or anti-HA antibody, respectively, and the amount of the second protein was measured in the total lysate (lane 2) and IP eluate (lane 4) by Western blotting. As shown in FIG. 2A, HA-NCK1 co-immunoprecipitated with FLAG-PTPlB. Likewise, as shown in FIG. 2B, FLAG-PTP1B co-immunoprecipitated with HA-NCK1. These results demonstrate that full-length PTP1B and full-length NCK1 proteins interact in vitro.

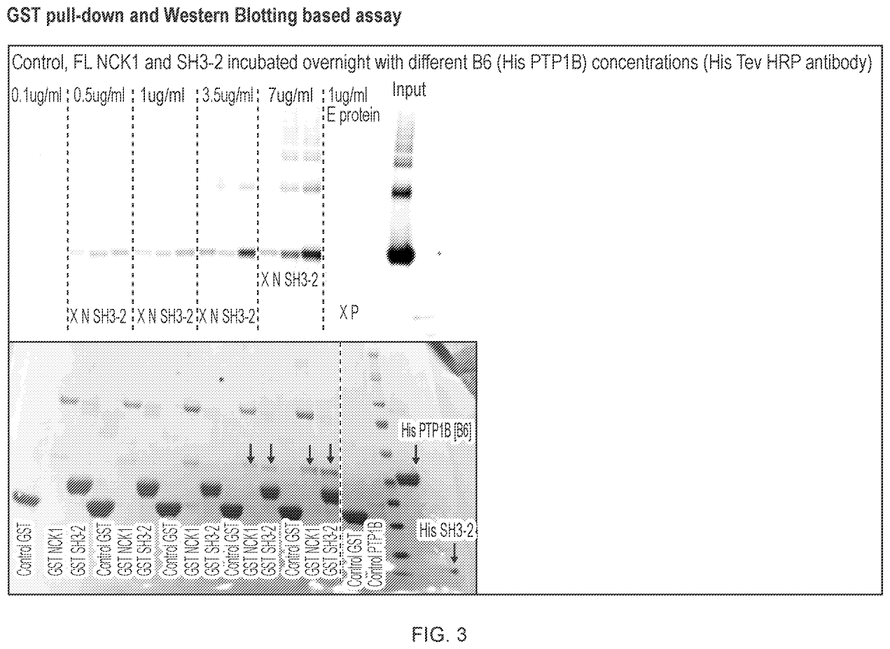

GST-tagged full-length NCK1, GST-tagged NCK1 SH3-2 domain, and His-tagged PTP1B were generated as described hereinabove. GST-tagged NCK1 and NCK1 SH3-2 domain were to bound to GSH-agarose beads and subsequently incubated with varying concentrations of HIS-tagged PTP1B. Non-loaded agarose beads were used as a control (“X” in FIG. 3). Following overnight incubation, the beads were washed three times, the proteins were eluted, and the amount of PTP1B in the eluate was measured by Western blotting. As shown in FIG. 3, PTP1B co-immunoprecipitated with both full length NCK1 (“N”) and with the SH3-2 domain of NCK1 (“SH3-2”). The PTP1B signal increased with increasing PTP1B concentration.

These results confirm that PTP1B interacts with full-length NCK1 and demonstrate that PTP1B binds the SH3-2 domain of NCK1. The interaction between PTP1B and the SH3-2 domain of NCK1 was further confirmed in different in vitro assays, including Isothermal Titration Calorimetry (ITC), Surface Plasmon Resonance (SPR) and cell-based assay.

This Example confirms that PTP1B and NCK1 form a complex and furthermore identifies the SH3-2 domain of NCK1 as the site of interaction between the two proteins.

### Example 2. Identification of Peptides that Disrupt the Interaction Between PTP1B and NCK1

This Examples describes the identification of peptides that disrupt the interaction between PTP1B and the SH3-2 domain of NCK1. Peptides were screened using an immunoprecipitation assay, which identified peptides that disrupt the interaction. Surface plasmon resonance (SPR) was also used to confirm that these peptides bind to full-length NCK1.

### Materials and Methods

Construction and Purification of GST constructs: Human NCK1 (NM_006153) human cDNA was obtained (Origene, MD, USA). GST fusion constructs (GST-only control, GST full length NCK1, GST all SH3 domains of NCK1, GST SH2 domain of NCK1, GST SH3-1, GST SH3-2 and GST SH3-3) were amplified, double digested (EcoRI and XhoI, New England Biolabs), ligated in to pGEX-6P-2 vector (#M2200S, New England Biolabs; #GE28-9546-50, Merck), transformed in to DH5α E. coli cells (#C2987H, NEB) and purified using a plasmid preparation kit according to manufacturer's instructions (#T1010, New England Biolabs). GST-constructs were transformed in to BL21-Gold E. coli cells (#230134, Agilent), and GST-fusion proteins were produced, extracted, and bound to GST beads as described in Example 1. Beads were washed three times with STE buffer and stored at 4 °C.

GST pull-down experiments for compounds screening against FLAG PTP1B interaction with SH3-2 domain of NCK1. GST-bound agarose beads of SH3-2 domain were incubated overnight at 4 °C with lysates from HEK293 cells overexpressing FLAG PTP1B protein, to gether with various peptide compounds (1 mM) to screen for inhibition of PTP1B-NCK1 binding. Following overnight incubation, agarose beads were three times washed in STE buffer, and proteins were eluted in SDS-Laemmli buffer by heating at 95 °C for 10 min. Samples were spun at 12,000 rpm for 2 min, and the liquid fraction was transferred in to 1 mL Eppendorf tubes. Proteins were separated and detected by chemiluminescent Western Blotting (PEQLAB, Germany) using anti-FLAG HRP antibody (#A8592, Sigma).

### Results and Discussion

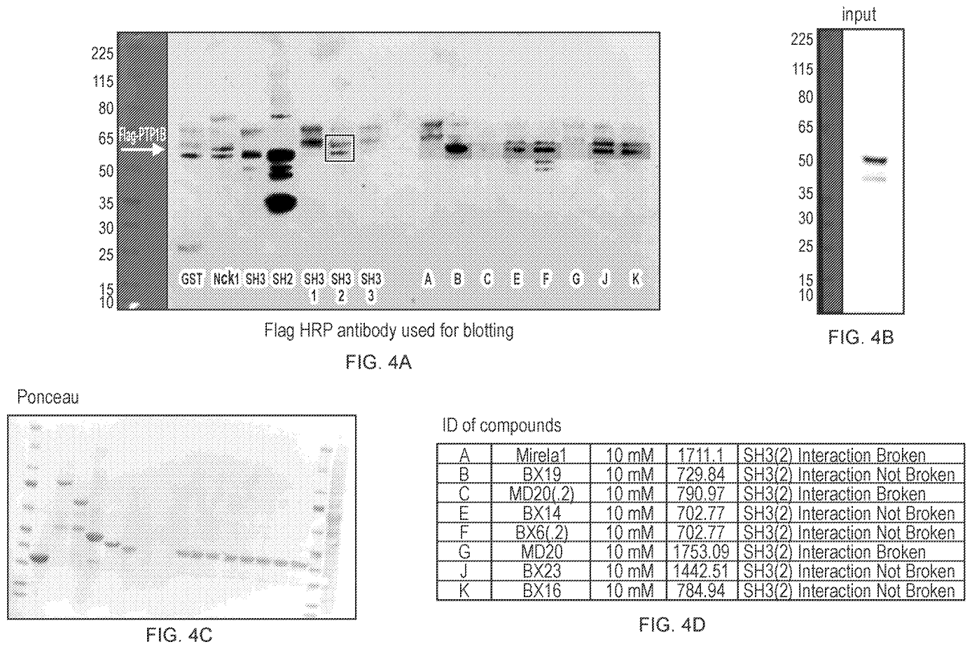

Peptides (1 mM) were screened using a co-immunoprecipitation assay for the ability to interrupt the interaction between the (GST-tagged) SH3-2 domain of NCK1 and (FLAG-tagged) PTP1B. As shown in FIGS. 4A-4D, the experiments described in this Example identified two peptides, MD17 (A) and MD20 (C and G), that disrupt the interaction between PTP1B and the SH3-2 domain of NCK1. These results confirmed that MD17 and MD20 bind to full-length NCK1, specifically, at SH3-2 domain.

### Example 3. Alanine-Scanning and Truncation Analysis of MD17, a Peptide that Disrupts the Interaction Between PTP1B and NCK1

This Example describes a study to identify critical structural features of the peptides that impact the function of the peptides. As described in Example 2, MD17 (a 15-mer) was identified as a peptide that binds to the SH3-2 domain of NCK1 and disrupts the interaction of PTP1B and NCK1. To explore which residues within the MD17 peptide that are critical for the activity of the MD17 peptide, alanine scanning and truncation analysis were performed on the MD17 peptide. Alanine scanning and truncation analysis were also performed on a 6-mer peptide (“MD20”) comprising the 6 C-terminal amino acid residues of MID17.

### Materials and Methods

All peptides were synthesized using Rink amide MBHA resin (0.68 mmol/g or 0.42 mmol/g loading). Peptides were generated using one of two methods.

Certain peptides were synthesized using a CEM LibertyBlue microwave-assisted peptide synthesizer. Couplings were conducted in N,N′-dimethylformamide (DMF) using N,N′-diisopropylcarbodiimide (DIC, 1 M solution) and OxymaPure (1 M solution) as coupling reagents, with Fmoc-AA-OH concentrations of 0.2 M. N-terminal Fluoren-9-ylmethyloxycarbonyl (Fmoc) deprotection was achieved using 20% (v/v) piperidine in DMF. N-terminal acetyl capping was achieved using a solution of DMF/acetic anhydride/N,N-diisopropylethylamine (70/20/5). Arg & His couplings were conducted at 70 °C and 55 °C, respectively, and Arg was double-coupled. All other amino acid couplings were conducted at 90 °C.

Alternatively, peptides were generated using a CEM MultiPep2 parallel synthesizer. Couplings were conducted in DMF using DIC (1 M) and OxymaPure (2 M) as coupling reagents, with Fmoc-AA-OH concentrations of 0.5 M. N-terminal Fmoc deprotection was achieved using 20% (v/v) piperidine in DMF. All couplings were conducted at 60 °C and double coupled as standard. Capping after each coupling was accomplished using 5% (v/v) acetic anhydride in DMF. Upon completion of synthesis and final deprotection, N-terminal acetyl capping was achieved using 10% (v/v) acetic anhydride in DMF.

Final peptides that did not contain Cys or Met were cleaved from the resin using a solution of trifluoroacetic acid/triisopropylsilane/H₂O (95/2.5/2.5) under mechanical shaking for 3 hours. Cys- and Met-containing peptides were cleaved using trifluoroacetic acid/triisopropylsilane/2,2′-(Ethylenedioxy)diethanethiol/H₂O (92.5/2.5/2.5/2.5). Upon evaporation of solvent, the solid crude peptide was precipitated by addition of ether.

Peptide purification was achieved using an Agilent preparative HPLC system with a Phenomenex Luna C18(2) 5 μm 21×250 mm 100 Å column. LCMS analysis of peptides was achieved using an Agilent 1200 system with a Phenomenex Luna C18(2) 5 μm 4.6×250 mm 100 Å column.

Isothermal titration calorimetry (ITC) was used to determine binding affinities for peptides against the SH3-2 domain of NCK1. Experiments were conducted at 25 °C using either a MicroCal iTC200 or MicroCal PEAQ-ITC calorimeter. The buffer used for all ITC experiments was 10 mM sodium phosphate, 100 mM sodium chloride (pH=8.0). A series of peptides (concentration 3-7.5 mM) were titrated in to NCK1 SH3-2 domain (concentration 0.125-0.3 mM) using the following conditions: one injection of 0.2 μL followed by 19 injections of 2 μL, 120-150 seconds spacing between injections, at 25 °C. The data were fit using analysis software provided by the manufacturer and affinity values were calculated. The best fitting occurred when the number of sites was between 0.8 and 1.2. The experiments were repeated twice for a total of three times, and the average binding affinity for each peptide was generated.

The sequences and structures of each of the tested peptides are detailed in Tables 3-12 below.

### Results and Discussion

The sequence and binding affinities of each of the tested peptides are detailed in Tables 3, 4, 5, and 6, below. In general, a peptide having a K_{D} of less than or equal to 300 μM was considered to have a good ability to bind NCK1 (Group A), a peptide having a K_{D} greater than 300 μM and less than or equal to 600 μM was considered to have a moderate ability to bind NCK1 (Group B), and a peptide having a K_{D} greater than 600 μM and less than or equal to 1,000 μM was considered to be a poor binder of NCK1 (Group C). “Group D” indicates that the peptide either had a K_{D} of greater than 1,000 μM, or that no binding was detected.

TABLE 3
Alanine scanning of the MD17 15-mer peptide

| | SEQ ID | | |
|---|---|---|---|
| Code | NO: | Peptide | K_{D} (μM) |
| MD17 | 10 | Ac-PPPEHIPPPPRPPKR-NH₂ | Group A |
| MSJ012 | 22 | Ac-APPEHIPPPPRPPKR-NH₂ | Group A |
| MSJ013 | 23 | Ac-PAPEHIPPPPRPPKR-NH₂ | Group A |
| MSJ014 | 24 | Ac-PPAEHIPPPPRPPKR-NH₂ | Group A |
| MSJ015 | 25 | Ac-PPPAHIPPPPRPPKR-NH₂ | Group A |
| MSJ016 | 26 | Ac-PPPEAIPPPPRPPKR-NH₂ | Group A |
| MSJ017 | 27 | Ac-PPPEHAPPPPRPPKR-NH₂ | Group A |
| MSJ018 | 28 | Ac-PPPEHIAPPPRPPKR-NH₂ | Group A |
| MSJ019 | 29 | Ac-PPPEHIPAPPRPPKR-NH₂ | Group A |
| MH001 | 30 | Ac-PPPEHIPPAPRPPKR-NH₂ | Group A |
| MH002 | 31 | Ac-PPPEHIPPPARPPKR-NH₂ | Group D |
| MH003 | 32 | Ac-PPPEHIPPPPAPPKR-NH₂ | Group A |
| MH004 | 33 | Ac-PPPEHIPPPPRAPKR-NH₂ | Group A |
| MH005 | 34 | Ac-PPPEHIPPPPRPAKR-NH₂ | Group D |
| MH006 | 35 | Ac-PPPEHIPPPPRPPAR-NH₂ | Group A |
| MH007 | 36 | Ac-PPPEHIPPPPRPPKA-NH₂ | Group D |

TABLE 4
Alanine scanning of the MD20 6-mer peptide

| | SEQ ID | | |
|---|---|---|---|
| Code | NO: | Peptide | K_{D} (μM) |
| MD20 | 8 | Ac-PRPPKR-NH₂ | Group A |
| MH008 | 37 | Ac-ARPPKR-NH₂ | Group D |
| MH009 | 38 | Ac-PAPPKR-NH₂ | Group A |
| MH010 | 39 | Ac-PRAPKR-NH₂ | Group D |
| MH011 | 40 | Ac-PRPAKR-NH₂ | Group D |
| MH012 | 41 | Ac-PRPPAR-NH₂ | Group B |
| MH013 | 42 | Ac-PRPPKA-NH₂ | Group D |

TABLE 5
Peptide truncation analysis

| | SEQ ID | | |
|---|---|---|---|
| Code | NO: | Peptide | K_{D} (μM) |
| MD20 | 8 | Ac-PRPPKR-NH₂ | Group A |
| MH014 | 43 | Ac-PKR-NH₂ | Group D |
| MH015 | 44 | Ac-PPK-NH₂ | Group D |
| MH016 | 45 | Ac-RPPK-NH₂ | Group D |
| MH017 | 46 | Ac-PPKR-NH₂ | Group D |
| MH018 | 47 | Ac-RPPKR-NH₂ | Group D |
| MH019 | 48 | Ac-PRP-NH₂ | Group D |
| MH020 | 49 | Ac-RPP-NH₂ | Group D |
| MH021 | 50 | Ac-PRPP-NH₂ | Group D |
| MH022 | 51 | Ac-PRPPK-NH₂ | Group D |
| MD18 | 9 | Ac-PPRPPKR-NH₂ | Group A |
| MH047 | 59 | Ac-APRPPKR-NH₂ | Group A |
| MH024 | 11 | Ac-PPPRPPKR-NH₂ | Group A |
| MH025 | 12 | Ac-PPPPRPPKR-NH₂ | Group A |
| MH026 | 13 | Ac-IPPPPRPPKR-NH₂ | Group A |
| MH027 | 14 | Ac-HIPPPPRPPKR-NH₂ | Group A |
| MH028 | 15 | Ac-EHIPPPPRPPKR-NH₂ | Group A |
| MH029 | 16 | Ac-PEHIPPPPRPPKR-NH₂ | Group A |
| MH030 | 17 | Ac-PPEHIPPPPRPPKR-NH₂ | Group A |

TABLE 6
Sliding Window Peptide Analysis

| | SEQ ID | | |
|---|---|---|---|
| Code | NO: | Peptide | K_{D} (μM) |
| MH035 | 52 | Ac-PPRPPK-NH₂ | Group D |
| MH036 | 53 | Ac-PPPRPP-NH₂ | Group D |
| MH037 | 54 | Ac-PPPPRP-NH₂ | Group D |
| MH038 | 55 | Ac-IPPPPR-NH₂ | Group D |
| MH039 | 56 | Ac-EHIPPP-NH₂ | Group D |
| MH040 | 57 | Ac-PPEHIP-NH₂ | Group D |
| MH041 | 58 | Ac-PPPRPPK-NH₂ | Group D |

The ITC experiments conducted show that a sequence of five or fewer amino acids in length is not tolerated (see Table 5). Alanine screening of MD17 revealed a core 6-mer sequence containing certain residues that appear important for NCK1-binding. In the context of the IVID17 15-mer peptide, the Pro, Pro, and Arg in positions 10, 13, and 15, respectively are required for the peptide to bind to the SH3-2 domain of NCK1 (see Table 3). The same three residues in the MD20 6-mer (corresponding to the Pro, Pro, and Arg in positions 1, 4, and 6, respectively) were likewise required for NCK1-binding in all of the peptides tested (see Table 4). Interestingly, the Pro in position 12 of MD17 was not required for binding in the context of the 15-mer (Table 3, 1v11004), although NCK1-binding was abolished when the Pro in the corresponding position of the 6-mer MID20 (position 3) was substituted for Ala. This may indicate that substitution of that proline (position 3 in MID20, position 12 in MD17) is tolerated better in longer peptides than in shorter peptides. In all of the relevant peptides tested, removing the Arg at position 15 in MD17 abolished binding of the peptide to bind to the SH3-2 domain of NCK1 (see MH013 in Table 4, MH022 in Table 5, and MH035 and MH041 in Table 6). The addition of an amino acid residue (e.g., Pro or Ala) to the N-terminus of MD20 has a positive effect on binding when compared to MD20. The addition of further residues to the N-terminal end did not have a detrimental effect on binding (see Table 5).

Thus, the present Example demonstrates that a core internal fragment of MD17 of six or seven residues (designated MD20 and MID18, respectively) retained the ability to bind to the SH3-2 domain of NCK1, each with a K_{D}<193 μM. Moreover, these results indicate that the Pro, Pro, and Arg residues at positions 1, 4, and 6, respectively, of MD20 (positions 2, 5, and 7 of MD18) are important for this binding activity. The requirement for Pro at position three of MD20 (position four of MID8) may vary depending on the length of the peptide (e.g., a Pro in that position may be required in a 6-mer, but not a 15-mer). These experiments also suggest that the residues at positions two and five of MD20 (or positions three and six, of MID18) can tolerate a substitution. The addition of an N-terminal amino acid to the core 6-mer sequence (e.g., Pro or Ala) improves binding to the SH3-2 domain of NCK1.

### Example 4. Screening of Additional NCK1-Binding Peptides

This example describes a study to identify additional peptides that bind to NCK-1 and potentially disrupt NCK1's interaction with PTP1B. In this study, additional single-substitution variants of MD18 (the 7-mer peptide identified as a core binding peptide in Example 3) were synthesized and analyzed for their ability to bind to NCK1's SH3-2 domain. Additional peptides, including peptides having additional amino acid residues at the N-terminal or C-terminal ends of the of the core 6/7-mer sequence or the 15-mer sequence, were also synthesized and analyzed for their ability to bind the SH3-2 domain of NCK1.

### Material and Methods

ITC binding assays were performed as described in Example 3, except that the 30-mer peptides listed in Table 12 and certain peptides in Table 13 were tested between one and three times.

### Results and Discussion

The sequence and binding affinities of each of the additional tested peptides are detailed in Tables 7-14, below. As in Example 3, in general, a peptide having a K_{D} of less than or equal to 300 μM was considered to have a good ability to bind NCK1 (Group A), a peptide having a K_{D} greater than 300 μM and less than or equal to 600 μM was considered to have a moderate ability to bind NCK1 (Group B), and a peptide having a K_{D} greater than 600 M was considered to be a poor binder of NCK1 (Group C). “Group D” indicates that the peptide either had a K_{D} of greater than 1,000 μM, or that no binding was detected. Sequences of the peptides MD 17, NM18, and MID20 are included in certain of the Tables below, for reference.

TABLE 7
Modified MD18/MD20 Peptides:

| | SEQ | | |
|---|---|---|---|
| Name | ID NO: | Peptide | K_{D} |
| MD20 | 8 | Ac-PRPPKR-NH₂ | [See Example 3] |
| MD18 | 9 | Ac-PPRPPKR-NH₂ | [See Example 3] |
| MH043 | 168 | H-Pyr-PRPPKR-NH₂ | Group A |
| MH044 | 169 | H-Pyr-RPPKR-NH₂ | Group D |
| MH045 | 8 | Ac-PRPPKR-NHEt | Group A |
| MH046 | 9 | Ac-PPRPPKR-NHEt | Group A |
| MH047 | 59 | Ac-APRPPKR-NH₂ | Group A |
| MH075 | 9 | H-PPRPPKR-NH₂ | Group A |
| MH076 | 60 | Ac-PPRPPKK-NH₂ | Group D |
| MH080 | 8 | H-PRPPKR-NH₂ | Group D |

TABLE 8
Tolerability of substitutions at position 1 of MD18

| | SEQ | | |
|---|---|---|---|
| Name | ID NO: | Peptide | K_{D} |
| MD18 | 9 | Ac-PPRPPKR-NH₂ | [See Example 3] |
| MH047 | 59 | Ac-APRPPKR-NH₂ | Group A |
| MH068 | 61 | Ac-RPRPPKR-NH₂ | Group A |
| MH069 | 62 | Ac-NPRPPKR-NH₂ | Group A |
| MH070 | 63 | Ac-CPRPPKR-NH₂ | Group A |
| MH071 | 64 | Ac-EPRPPKR-NH₂ | Group A |
| MH072 | 65 | Ac-HPRPPKR-NH₂ | Group A |
| MH073 | 66 | Ac-FPRPPKR-NH₂ | Group A |
| MH074 | 67 | Ac-SPRPPKR-NH₂ | Group A |
| MH083 | 124 | Ac-DPRPPKR-NH₂ | Group A |
| MH086 | 125 | Ac-QPRPPKR-NH₂ | Group A |
| MH091 | 126 | Ac-GPRPPKR-NH₂ | Group A |
| MH095 | 127 | Ac-IPRPPKR-NH₂ | Group A |
| MH099 | 128 | Ac-LPRPPKR-NH₂ | Group B |
| MH103 | 129 | Ac-KPRPPKR-NH₂ | Group A |
| MH109 | 130 | Ac-MPRPPKR-NH₂ | Group A |
| MH113 | 131 | Ac-TPRPPKR-NH₂ | Group A |
| MH117 | 132 | Ac-WPRPPKR-NH₂ | Group A |
| MH119 | 133 | Ac-YPRPPKR-NH₂ | Group A |
| MH123 | 134 | Ac-VPRPPKR-NH₂ | Group A |

TABLE 9
Tolerability of substitutions at position 3 of MD18 (position 2 of MD20)

| Name | SEQ ID NO: | Peptide | K_{D} |
|---|---|---|---|
| MD18 | 9 | Ac-PPRPPKR-NH₂ | [See Example 3] |
| MH055 | 68 | Ac-PPKPPKR-NH₂ | Group A |
| MH066¹ | 69 | Ac-PPFPPKR-NH₂ | Group A |
| MH057 | 70 | Ac-PPHPPKR-NH₂ | Group A |
| MH058 | 71 | Ac-PPEPPKR-NH₂ | Group A |
| MH059 | 72 | Ac-PPSPPKR-NH₂ | Group A |
| MH060 | 73 | Ac-PPNPPKR-NH₂ | Group A |
| MH061 | 74 | Ac-PPCPPKR-NH₂ | Group A |
| MH084 | 113 | Ac-PPDPPKR-NH₂ | Group A |
| MH087 | 114 | Ac-PPQPPKR-NH₂ | Group A |
| MH092 | 115 | Ac-PPGPPKR-NH₂ | Group C |
| MH100¹ | 116 | Ac-PPLPPKR-NH₂ | Group A |
| MH096¹ | 117 | Ac-PPIPPKR-NH₂ | Group A |
| MH106 | 118 | Ac-PPPPPKR-NH₂ | Group A |
| MH110¹ | 119 | Ac-PPMPPKR-NH₂ | Group A |
| MH114 | 120 | Ac-PPTPPKR-NH₂ | Group A |
| MH056¹ | 121 | Ac-PPWPPKR-NH₂ | Group B |
| MH120¹ | 122 | Ac-PPYPPKR-NH₂ | Group A |
| MH124¹ | 123 | Ac-PPVPPKR-NH₂ | Group A |

¹Tested peptides had <90% purity; predicted to be mixture of conformers.

TABLE 10
Tolerability of substitutions at position 4 of MD18 in a 7-mer

| Name | SEQ ID NO: | Peptide | K_{D} |
|---|---|---|---|
| MD18 | 9 | Ac-PPRPPKR-NH₂ | [See Example 3] |
| MH062 | 75 | Ac-PPRRPKR-NH₂ | Group B |
| MH067 | 76 | Ac-PPRFPKR-NH₂ | Group C |
| MH093 | 86 | Ac-PPRGPKR-NH₂ | Group D |
| MH097 | 87 | Ac-PPRIPKR-NH₂ | Group A |
| MH101 | 88 | Ac-PPRLPKR-NH₂ | Group B |
| MH104 | 89 | Ac-PPRKPKR-NH₂ | Group B |
| MH108 | 90 | Ac-PPRSPKR-NH₂ | Group D |
| MH127 | 91 | Ac-PPRNPKR-NH₂ | Group D |
| MH128 | 92 | Ac-PPRDPKR-NH₂ | Group D |
| MH129 | 93 | Ac-PPRCPKR-NH₂ | Group D |
| MH130 | 94 | Ac-PPREPKR-NH₂ | Group D |
| MH131 | 95 | Ac-PPRQPKR-NH₂ | Group D |
| MH132 | 96 | Ac-PPRHPKR-NH₂ | Group D |
| MH111 | 97 | Ac-PPRMPKR-NH₂ | Group D |
| MH115 | 98 | Ac-PPRTPKR-NH₂ | Group B |
| MH118 | 99 | Ac-PPRWPKR-NH₂ | Group D |
| MH121 | 100 | Ac-PPRYPKR-NH₂ | Group B |
| MH125 | 101 | Ac-PPRVPKR-NH₂ | Group A |

TABLE 11
Tolerability of substitutions at position 6 of MD18 (position 5 of MD20)

| Name | SEQ ID NO: | Peptide | K_{D} |
|---|---|---|---|
| MD18 | 9 | Ac-PPRPPKR-NH₂ | [See Example 3] |
| MH048 | 77 | Ac-PPRPPRR-NH₂ | Group A |
| MH049² | 78 | Ac-PPRPPHR-NH₂ | Group A |
| MH050 | 79 | Ac-PPRPPWR-NH₂ | Group A |
| MH051 | 80 | Ac-PPRPPER-NH₂ | Group A |
| MH052 | 81 | Ac-PPRPPSR-NH₂ | Group A |
| MH053 | 82 | Ac-PPRPPNR-NH₂ | Group A |
| MH054 | 83 | Ac-PPRPPCR-NH₂ | Group A |
| MH094 | 102 | Ac-PPRPPGR-NH₂ | Group C |
| MH098 | 103 | Ac-PPRPPIR-NH₂ | Group A |
| MH102 | 104 | Ac-PPRPPLR-NH₂ | Group A |
| MH105 | 105 | Ac-PPRPPFR-NH₂ | Group B |
| MH085 | 106 | Ac-PPRPPDR-NH₂ | Group B |
| MH112 | 107 | Ac-PPRPPMR-NH₂ | Group A |
| MH088 | 108 | Ac-PPRPPQR-NH₂ | Group A |
| MH107 | 109 | Ac-PPRPPPR-NH₂ | Group A |
| MH116 | 110 | Ac-PPRPPTR-NH₂ | Group A |
| MH122 | 111 | Ac-PPRPPYR-NH₂ | Group A |
| MH126 | 112 | Ac-PPRPPVR-NH₂ | Group A |

²Peptide was tested at 87% purity

TABLE 12
Testing peptides with N- or C-terminal extensions

| | SEQ | | |
|---|---|---|---|
| Name | ID NO: | Peptide | K_{D} |
| MD18 | 9 | Ac-PPRPPKR-NH₂ | [See |
| | | | Example 3] |
| MH063 | 18 | Ac-PPRPPKRI-NH₂ | Group A |
| MH064 | 19 | Ac-PPRPPKRIL-NH₂ | Group A |
| MH065 | 20 | Ac-PPRPPKRILE-NH₂ | Group A |
| MH077 | 21 | Ac-PPPEHIPPPPRPPKRILE-NH₂ | Group A |
| MH089 | 135 | Ac-PPPEHIPPPPRPPKRILEPH-NH₂ | Group A |
| MH134 | 137 | Ac-PPPEHIPPPPRPPKRILEPHNGKCR-NH₂ | Group A |
| MH137 | 141 | Ac- | Group A |
| | | PPPEHIPPPPRPPKRILEPHNGKCREFFPN- | |
| | | NH₂ | |
| MH090 | 136 | Ac-HEDLEPPPEHIPPPPRPPKR-NH₂ | Group A |
| MH135 | 142 | Ac-WKELSHEDLEPPPEHIPPPPRPPKR-NH₂ | Group A |
| MH136 | 143 | Ac- | Group A |
| | | SVQDQWKELSHEDLEPPPEHIPPPPRPPKR- | |
| | | NH₂ | |

TABLE 13
Tolerability of substitutions at position 8 of the 8-mer MH063

| Name | SEQ ID NO: | Peptide | K_{D} |
|---|---|---|---|
| MH063 | 18 | Ac-PPRPPKRI-NH₂ | Group A |
| MH138 | 144 | Ac-PPRPPKRA-NH₂ | Group A |
| MH139 | 145 | Ac-PPRPPKRR-NH₂ | Group A |
| MH140 | 146 | Ac-PPRPPKRN-NH₂ | Group A |
| MH141 | 147 | Ac-PPRPPKRD-NH₂ | Group A |
| MH142 | 148 | Ac-PPRPPKRC-NH₂ | Group A |
| MH145 | 151 | Ac-PPRPPKRG-NH₂ | Group A |
| MH146 | 152 | Ac-PPRPPKRH-NH₂ | Group A |
| MH147 | 153 | Ac-PPRPPKRL-NH₂ | Group A |
| MH148 | 154 | Ac-PPRPPKRK-NH₂ | Group A |
| MH149 | 155 | Ac-PPRPPKRM-NH₂ | Group A |
| MH150 | 149 | Ac-PPRPPKRF-NH₂ | Group A |
| MH151 | 156 | Ac-PPRPPKRP-NH₂ | Group B |
| MH152 | 150 | Ac-PPRPPKRS-NH₂ | Group A |
| MH154 | 157 | Ac-PPRPPKRW-NH₂ | Group A |
| MH155 | 158 | Ac-PPRPPKRY-NH₂ | Group A |

TABLE 14
Testing of exemplary control peptides

| Name | SEQ ID NO: | Peptide | K_{D} |
|---|---|---|---|
| MH081³ | 138 | H-PAVPPRP-NH₂ | Group D |
| MH082³ | 138 | Ac-PAVPPRP-NH₂ | Group D |
| MH133 | 139 | Ac-SPAKGEPSLPEK-NH₂ | Group D |

³Tested peptides had <90% purity; predicted to be mixture of conformers.

The results of Example 3 demonstrated that the addition of either proline or alanine to the N-terminus of the core MD20 6-mer sequence improved binding to SH3-2. The results of the further ITC experiments in this Example demonstrate that substitution of the Pro residue at position 1 of MD18 for various different amino acids is well tolerated, with most substitutions having similar binding affinity to MD18 (see Table 8). In further testing, the N-terminal proline in position 1 of MD18 was replaced with a pyroglutamic acid moiety (see Table 7, MH043), which comprises a five-membered ring (analogously to proline). This peptide was able to bind NCK1 with comparable efficiency as MID18.

The results of Example 3 demonstrated that the proline at position 2 of MD18 (position 1 of the MD20 6-mer) is important for binding to the SH3-2 domain of NCK1. The results of the further ITC experiments in this Example demonstrate that replacing this proline in MD20 with a pyroglutamic acid moiety (see Table 7, MH044) results in undetectable binding to NCK1. Additionally, a further variant of MD20 with a free N-terminus was also synthesized and tested, and had a K_{D} falling in to Group D (see Table 7, MH080). These results suggest that, in peptides in which the amino acid at position 1 of MD20 is the N-terminal amino acid of the peptide, the additional carbonyl moiety attached to the amino acid residue at position 1 of MD20 is important for NCK1-binding.

The results of Example 3 demonstrated that substituting the Arg at position 3 of MD18 (position 2 of the MD20) with alanine is well-tolerated. The results of the further ITC experiments in this Example demonstrate that substitutions with various other amino acids are also well-tolerated (see Table 9). Substituting the Arg at this position with any amino acid, including uncharged, bulky, or charge-flipped amino acids, did not abolish binding to NCK1. However, substitution with tryptophan or glycine somewhat decreased binding to NCK1, indicating that increased bulk (Trp) or increased flexibility (Gly) is not favorable in this position.

The results of prior Example 3 indicated that the proline at position 4 of MD18 (position 3 of the MD20 6-mer) is important for binding to the SH3-2 domain of NCK1 in the context of a 6-mer, but not a 15-mer. The results of the further ITC experiments demonstrate that, in the context of a 7-mer peptide, replacing the proline in position 4 of MID18 is not well tolerated, with some exceptions. Replacing the proline in this position with valine or isoleucine was well tolerated in a 7-mer. The results also demonstrate that replacing the proline in this position with an arginine, leucine, lysine, threonine, or tyrosine somewhat reduced, but did not abolish, peptide binding to the SH3-2 domain of NCK1 (see Table 10). Replacing the proline in this position with phenylalanine further reduced (but did not abolish) binding to the SH3-2 domain of NCK1 (see Table 10). Replacing the proline in this position with a glycine, serine, asparagine, aspartic acid, cysteine, glutamic acid, glutamine, histidine, methionine, or tryptophan abolished detectable binding to the SH3-2 domain of NCK1. No substitutions at this position resulted in a greater binding affinity than MID18 (see Table 10). These results suggest that certain, but not all, substitutions of the Pro in this position (position 3 in the 6-mer) are tolerated in a 7-mer.

The results of Example 3 demonstrated that substituting the Lys at position 6 of MD17 (position 5 of the 6-mer MD20) with alanine is well-tolerated. The data from the further ITC experiments in this Example demonstrate that substitutions with various other amino acids are also well-tolerated (see Table 11). Substituting the Lys at this position with uncharged, bulky, or charge-flipped amino acids did not abolish binding to NCK1. However, substitution of the lysine in this position with glycine reduced (but did not abolish) binding, as compared to MID18.

As shown in Table 12, the addition of one, two, or three amino acids to the C-terminal end of MD18 did not reduce binding to NCK1. Additionally, the addition of three, five, ten, or fifteen C-terminal amino acids to the end of the 15-mer peptide MD17 (thereby forming an 18-mer, a 20-mer, a 25-mer, and a 30-mer, respectively) did not reduce binding to NCK1. The addition of five, ten, or fifteen N-terminal amino acids to the end of the 15-mer peptide MD17 (thereby forming a 20-mer, 25-mer, and a 30-mer, respectively) also did not reduce binding to NCK1.

As shown in Table 13, an 8-mer (comprising the amino acid sequence of SEQ ID NO: 9, and further comprising a C-terminal isoleucine residue) was shown to effectively bind NCK1. Additionally, the replacement of the C-terminal isoleucine residue with various charged, uncharged, or hydrophobic amino acid residues did not reduce binding to NCK1 (see Table 13). Replacement of the C-terminal isoleucine residue with proline reduced, but did not abolish, binding to NCK1.

Three exemplary control peptides shown in Table 14 were tested for their ability to bind NCK1. As summarized in Table 14, none of the three tested control peptides detectably bound NCK1.

### Example 5. Pharmacokinetic Analysis of MD20

This Examples describes in vitro and in vivo experiments performed to assess the stability, cell permeability, and bioavailability of MD20.

### Materials and Methods

Microsomal Metabolic Stability (human liver microsomes): Human liver microsomes (final protein concentration 0.5 mg/mL), 0.1 M phosphate buffer pH 7.4 and test compound MD20 (final substrate concentration 1 μM; final DMSO concentration 0.25%) were pre-incubated at 37 °C prior to the addition of NADPH (final concentration 1 mM) to initiate the reaction. A minus cofactor control incubation was included for each compound tested where 0.1 M phosphate buffer pH 7.4 was substituted for NADPH (“minus NADPH”).

Each compound was incubated for 0, 5, 15, 30 and 45 min. The control (minus NADPH) was incubated for 45 min only. The reactions were stopped by transferring incubate in to acetonitrile at the appropriate time points, in a 1:3 ratio. The termination plates were centrifuged at 3,000 rpm for 20 min at 4 °C to precipitate the protein. Following protein precipitation, the sample supernatants were combined in cassettes of up to 4 compounds, internal standard was added and samples were analyzed using Cyprotex generic LC-MS/MS conditions. The time point at which 30-70% of parent had degraded was then investigated at 3 different levels of metabolite profiling and/or identification. The intrinsic clearance (CLint) of the test compound was calculated.

Permeability (Caco-2 assay): Caco 2 cells were used as an in vitro model of the human intestinal epithelium and to permit assessment of the intestinal permeability of potential drugs. Test compound was added to either the apical or basolateral side of a confluent monolayer of Caco 2 cells and permeability was measured by monitoring the appearance of the test compound on the opposite side of the monolayer using LC-MS/MS. When performing a bi-directional assay, the efflux ratio (ER) was calculated from the ratio of B-A and A-B permeabilities.

Briefly, Caco 2 cells obtained (ATCC) were used between passage numbers 40 and 60. Cells were seeded on to Transwell plates at 1×10⁵ cells/cm². The cells were cultured in DMEM and media was changed every two or three days. On day 20, the permeability study was performed. Cell culture and assay incubations were carried out at 37 °C in an atmosphere of 5% CO₂ with a relative humidity of 95%. On the day of the assay, the monolayers were prepared by rinsing both apical and basolateral surfaces twice with Hanks Balanced Salt Solution (HBSS) at the desired pH warmed to 37 °C. Cells were then incubated with HBSS at the desired pH in both apical and basolateral compartments for 40 min to stabilise physiological parameters.

The dosing solutions were prepared by diluting test compound with assay buffer to give the desired test compound incubation concentration (typically 10 μM). DMSO was used at a final concentration of ≤1% v/v. The fluorescent integrity marker lucifer yellow was also included in the dosing solution. Analytical standards were prepared from test compound DMSO dilutions and transferred to buffer, maintaining a ≤1% v/v DMSO concentration. Typical assay buffer was supplemented HBSS pH 7.4, but a range of other buffers and pH values may be used.

To assess of A-B permeability, HBSS was removed from the apical compartment and replaced with test compound dosing solution. The apical compartment insert was then placed in to a companion plate containing fresh buffer (containing <1% v/v DMSO). For assessment of B-A permeability, HBSS was removed from the companion plate and replaced with test compound dosing solution. Fresh buffer (containing <1% v/v DMSO) was added to the apical compartment insert, which was then placed in to the companion plate.

At 120 min, the apical compartment inserts and the companion plates were separated and apical and basolateral samples were diluted for analysis.

Test and control compounds were quantified by LC-MS/MS cassette analysis using a 7-point calibration with appropriate dilution of the samples. Cyprotex generic analytical conditions were used. The starting concentration (C₀) was determined from the dosing solution and the experimental recovery was calculated from C₀ and both apical and basolateral compartment concentrations.

Data Analysis: The permeability coefficient (Pₐₚₚ) for each compound was calculated from the following equation:

Pₐₚₚ=((dQ/dt)/(C₀×A))
where dQ/dt represents the rate of permeation of the drug across the cells, C₀ represents the donor compartment concentration at time zero and A represents the area of the cell monolayer. C₀ was obtained from analysis of the dosing solution.

For bi directional experiments, an efflux ratio (ER) was calculated from mean A-B and B-A data using the following equation:

ER=P_{app(B-A)}/P_{app(A-B)}.

Four control compounds were screened alongside the test compounds, typically: atenolol (human absorption 50%), antipyrine (human absorption 97%), talinolol (a P-glycoprotein substrate) and estrone 3-sulfate (a BCRP substrate).

Biodistribution: In vivo pharmacokinetic assessments of biodistribution of MD20 were performed in C57/BL6 male mice. All animals were maintained under a 12 h light/dark cycle with free access to food and water, where temperature and humidity was controlled according to UK Home Office regulations. MD20 compound was dissolved in PBS and administered to animals via intraperitoneal (IP) injection at a dose of 30 mg/kg. Mice were sacrificed at eight different time points (0.25, 0.5, 1, 3, 4, 6, 8 and 24 h house post-dosing; n=3 animals per time point, 24 animals in total). Terminal blood (in EDTA-containing tubes for plasma), perfused liver, muscle, adipose, kidney and brain samples were collected and kept on dry ice. Blood samples were centrifuged (4 °C, 21,100 g for 5 min) and the resulting plasma was transferred in to corresponding tubes. All samples were analyzed using LC-MS and appropriate methods.

### Results and Discussion

The stability of the peptide MD20 was measured using a microsomal metabolic stability assay as described hereinabove. MD20 was found to have low intrinsic clearance (CL int value of 4.90) in human liver microsomes, indicating that MD20 is predicted to be very stable.

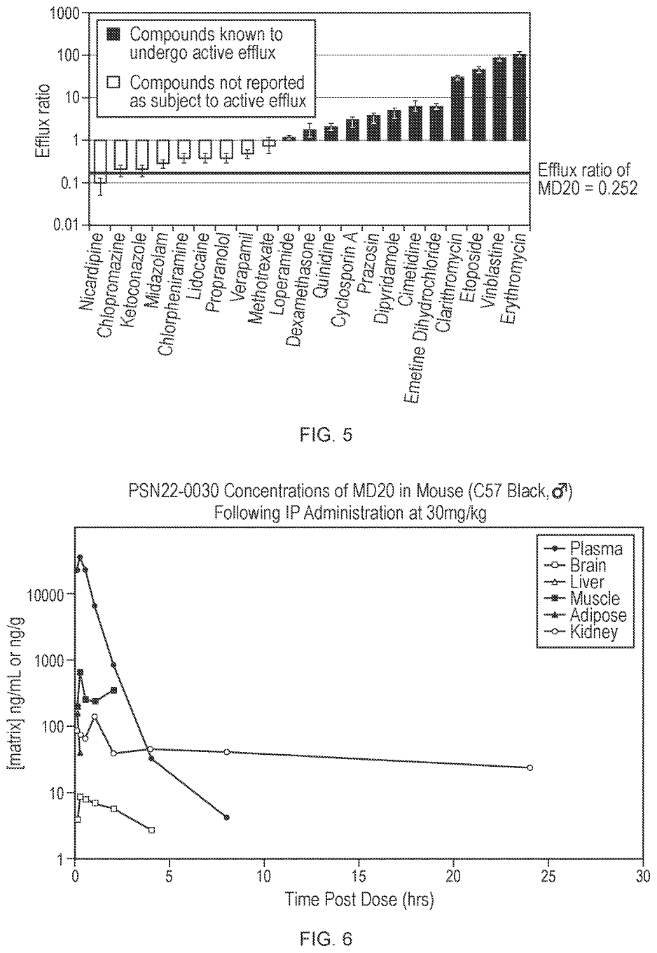

Permeability of MD20 was measured using a Caco-2 cell assay as described hereinabove. MD20 was found to be in the lower range (efflux ratio 0.252; FIG. 5), yet was still able to permeate the cells at the same rate as some antihypertensive drugs (e.g., nicardipine).

In vivo biodistribution of MD20 following IP administration to mice was analyzed as described hereinabove. MD20 was detected in most tissues and plasma up to 5 h post-administration, and even longer in other tissues, e.g., kidney (FIG. 6). No MD20 was detected in the liver, which is suggestive of either a very fast hepatic metabolism of MD20 or an inability of the test compound to enter liver cells. The nature of compound as a peptide may indicate that the former is more likely. Taken to gether, these pharmacokinetic analyses indicate that MD20 presents good stability and bioavailability in vivo, and that the compound can permeate cells.

Thus, this Example demonstrates that MD20 is considered to be a stable compound based on in vitro assay results and has good bioavailability in all tissues tested in vivo except liver. Further, MD20 is able to permeate cells comparably to some anti-hypertensive drugs that are commercially available.

### Example 6. Pharmacodynamic and Liver Toxicity Study of MD17 and MD20

This Example demonstrates the pharmacodynamics and safety profile of the MD17 and MD20 peptides. To assess the safety profile of MD17 and MD20 in vivo, these peptides were administered to mice for 28 days via daily IP administration which were subsequently assessed for physiological, metabolic, and liver signs of toxicity.

### Materials and Methods

Animals: C57BL/6 male mice (−3 months) were purchased from Charles River, UK. All animals were housed and tested following European (Directive on the Protection of Animals used for Scientific Purposes, 2010/63/EU) and UK Home Office regulations. Experiments were approved by the University Ethics Board and carried out in accordance with the Animal (Scientific Procedures) Act 1986 and following ARRIVE guidelines.

Treatments: Mice were randomized in to the five experimental groups: Control (n=9); MD17, 15 mg/kg (n=6); MD17, 30 mg/kg (n=6); MD20, 15 mg/kg (n=6); and MD20, 30 mg/kg (n=6). MD17 and MD20 compounds were administered to mice once daily via intraperitoneal (IP) injection for a period of 28 consecutive days at a dose of either 15 or 30 mg/kg. Peptides were kept in −20 °C and dissolved in sterile phosphate buffered saline (PBS) freshly every day before the injection. Control mice received a daily PBS injection. Three mice died during the study, and the final number of animals in each group was: Control (n=9); MD17, 15 mg/kg (n=5); MD17, 30 mg/kg, (n=6); MD20, 15 mg/kg (n=5); and MD20, 30 mg/kg (n=5). All mice had ad libitum access to food and water and were kept in a 12 h light/dark cycle.

Bodyweights and body mass composition: body weights were recorded during the study and fat and lean mass composition was analysed using an EchoMRI scanner (EchoMedical Systems, Houston, TX, U.S.A.).

Fasted glucose: All animals were 5 h-fasted and tail glucose measured using an AlphaTRAK 2 glucose strips and glucometer (Abbot, UK).

Serum assays: Trunk-derived blood was collected from 5 h-fasted mice in to serum separator microtubes and kept at −80 °C. Serum was used for insulin (#EZRMI-13K, Merck), leptin (#90030, Crystal Chem), free fatty acids (FFA; #mak044, Sigma-Aldrich), alanine and aspartate aminotransferases (ALT and AST; #ab105134 and #ab105135, Abcam) analyses.

Statistical analyses: Data were expressed as means±SEM and were analysed using one- or two-way ANOVA with Bonferroni post hoc tests, as appropriate. GraphPad Prism (V8, GraphPad Software, Inc., San Diego, California, USA) was used for analyses. P-values <0.05 were considered significant.

### Results and Discussion

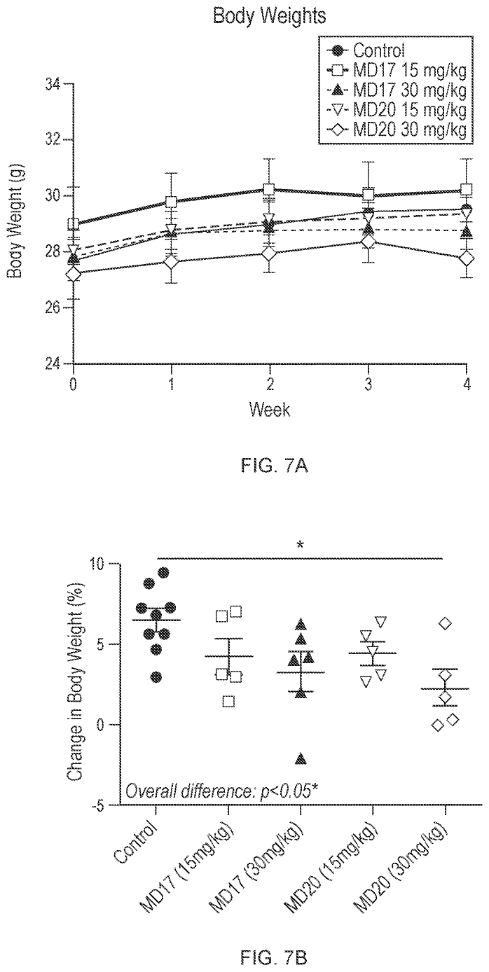

Mice were administered 15 mg/kg or 30 mg/kg daily doses of MD17 or MD20 for 28 days as described hereinabove and subsequently assessed for signs of physiological, metabolic, and liver toxicity. No statistically significant differences in body weights over the 4 weeks of treatments were detected between the treatment groups (FIG. 7A). However, individual analyses of percentage change in body weight pre- versus post-treatment revealed a significant decrease in % change following daily administration of 30 mg/kg MD20, as compared to the control group (FIG. 7B).

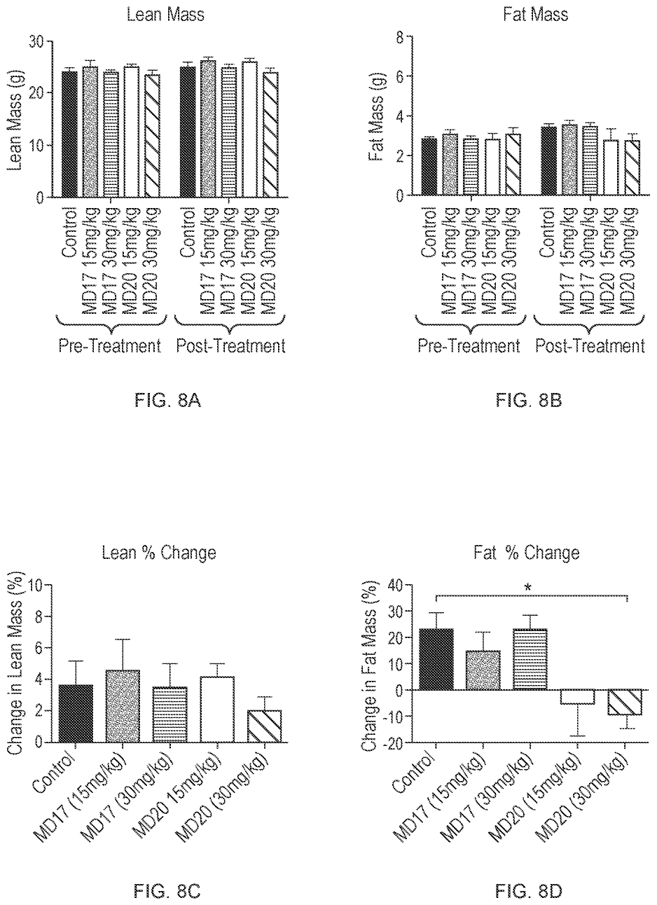

No statistically significant differences were detected in lean or fat mass composition as determined using Echo MRI scan across the animal groups (FIGS. 8A-8B). However, similarly to % change in body weights, % change in fat mass was also decreased in mice treated with MD20 (30 mg/kg), as compared to the control group (FIG. 8D).

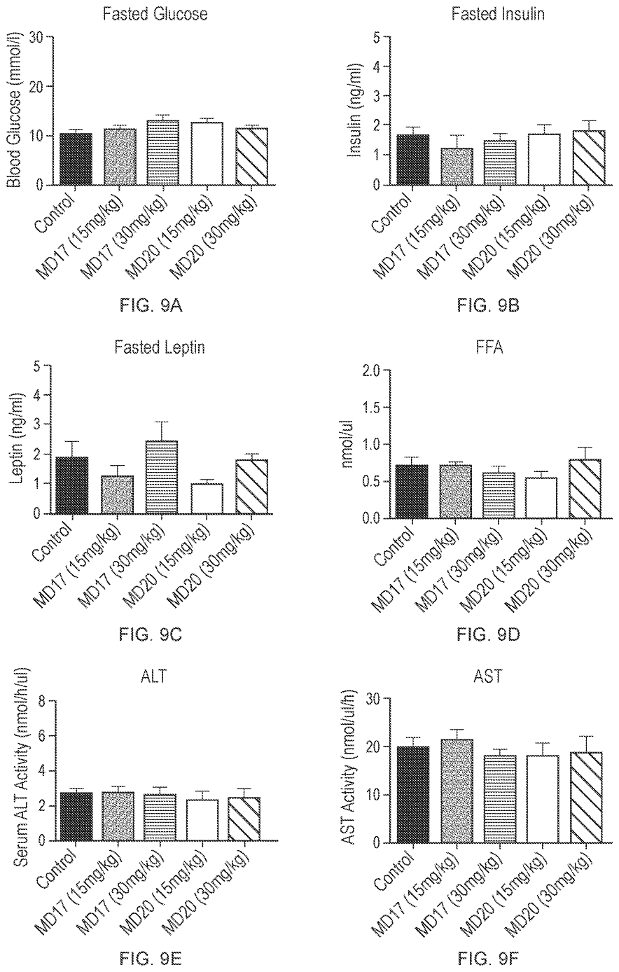

Mice were fasted for five hours at the conclusion of the experiment, and blood and serum samples were used to assess metabolic and liver toxicity. No significant differences were observed between treatment groups in a 5-hour fasted analysis of basal glucose levels (FIG. 9A), or in 5-hour fasted analyses of serum levels of insulin (FIG. 9B), leptin (FIG. 9C), free fatty acids (FIG. 9D), or liver toxicity markers ALT and AST (FIGS. 9E-9F).

Thus, this Example demonstrates that MD17 and MD20 daily IP administration for 28 days did not induce any obvious signs of physiological or hepatic toxicity (e.g., in ALT and AST analyses) in mice at the doses tested. Additionally, body weight and fat mass were lowered in mice following daily IP administration of 30 mg/kg MD20, which suggested further testing of MD20 in mouse models of obesity and type 2 diabetes.

### Example 7. Effects of MD20 in a High-Fat Diet-Induced Obesity and Type 2 Diabetes Model

This Example demonstrates the efficacy of the MD20 peptide in a relevant obesity and type 2 diabetes model. To test the effects of the MD20 peptide in vivo, the peptide was administered to a high-fat diet-induced obesity and type 2 diabetes model.

Obesity is the major risk factor for developing type 2 diabetes and accounts for ~90% of all type 2 diabetes cases. High fat diet (HFD)-induced obesity and type 2 diabetes mouse model is the most relevant model mimicking obesity, insulin resistance and type 2 diabetes associated with a Westernized lifestyle, which includes unhealthy eating, excessive caloric intake, and a sedentary way of life in humans. Hence, the model is considered to be the most relevant mouse model for mimicking early to mid-stage obesity and type 2 diabetes progression patterns in vivo (Wang and Liao (2012) METHODS MOL. BIOL. 421-433; Reuter (2007) DRUG DISCOV. TODAY DIS. MODELS 4(1): 3-8). The gold standard for diet-induced obesity model is considered to be male C57BL/6J mice placed on ad libitum access to high-fat diet, which leads to gradual development of obesity and type 2 diabetes phenotypes, associated with worsening of insulin sensitivity, glucose tolerance, hyperglycemia, and impaired lipid metabolism (Reuter, 2007).

### Materials and Methods

Animals: All animals were housed and tested following European (Directive on the Protection of Animals used for Scientific Purposes, 2010/63/EU) and UK Home Office regulations. Experiments were approved by the University Ethics Board and carried out in accordance with the Animal (Scientific Procedures) Act 1986 and following ARRIVE guidelines. C57BL/6 male mice (~3-4 months) were purchased from Charles River, UK. All mice received high fat and cholesterol diet (42% fat plus 0.2% cholesterol, #TD.88137, Envigo, US) to induce experimental type 2 diabetes and obesity phenotype. All mice had ad libitum access to food and water and were kept in a 12 h light/dark cycle.

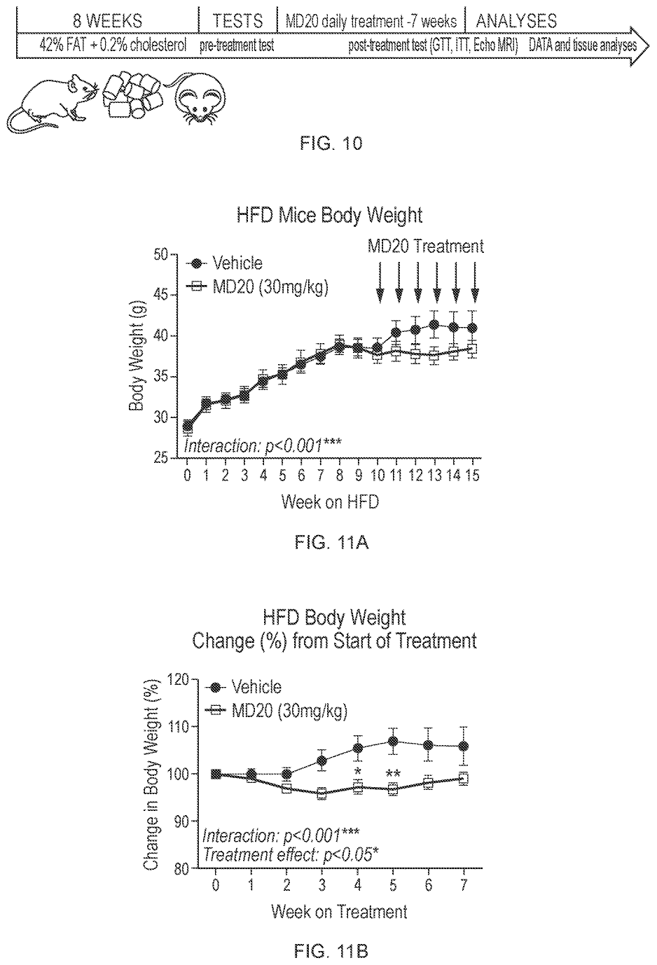

Treatments: The experimental protocol is summarized in FIG. 10. Briefly, after 8 weeks of high fat/high cholesterol diet feeding, mice were counterbalanced according to their body weight in to control (n=10) or MD20 30 mg/kg (n=10) groups. MD20 compound (30 mg/kg) was administered once daily via intraperitoneal (IP) injection for 7 weeks, whereas control mice received daily IP injection of vehicle (PBS). MD20 was kept at −20 °C and dissolved in sterile phosphate buffered saline (PBS) freshly every day before the injection. Three mice died during the study, and the final number of animals was: control (n=9) and MD20 30 mg/kg (n=8).

Bodyweights and body mass composition: Bodyweights were recorded during the study. Fat mass and lean mass composition were analysed using an EchoMRI scanner (EchoMedical Systems, Houston, TX, U.S.A.).

Metabolic measurements: All animals were 5 h-fasted before metabolic testing. Tail blood glucose was measured using AlphaTRAK 2 glucose strips and glucometer (Abbot, UK). Glucose tolerance tests (GTT) and insulin tolerance tests (ITT) were performed by IP injection of 2 mg/g of glucose (GTT) or 0.6 mU/g insulin (ITT), and glucose levels were measured at 15, 30, 60 and 90 min post-injection.

Serum and liver assays: Trunk-derived blood was collected from 5 h-fasted mice into serum separator microtubes and kept at −80 °C. Serum was used for insulin (#EZRMI-13K, Merck), leptin (#90030, Crystal Chem), free fatty acids (FFA; #mak044, Sigma-Aldrich), cholesterol (#MAK043, Sigma Aldrich), alanine, and aspartate aminotransferase (ALT and AST; ab105134 and ab105135, Abcam) analyses. Serum and liver triglycerides were determined using triglyceride reagents (#T2449, #F6428, #G7793, Sigma Aldrich).

Statistical analyses: Data were reported as means±SEM and analysed using one- or two-way ANOVA with Bonferroni post hoc tests, as appropriate. GraphPad Prism (V8, GraphPad Software, Inc., San Diego, California, USA) was used for statistical analyses. P-values <0.05 were considered significant.

### Results and Discussion

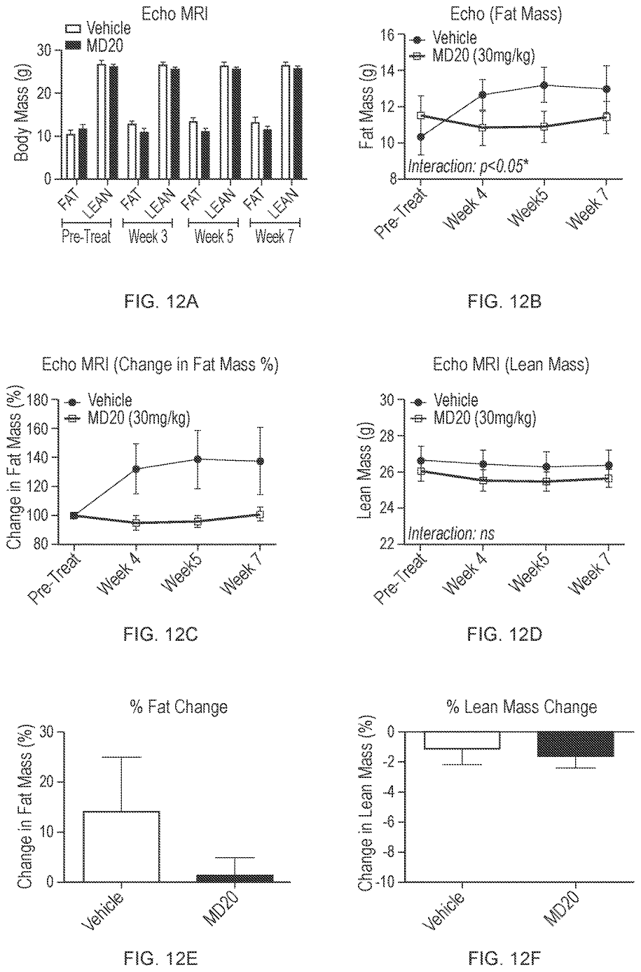

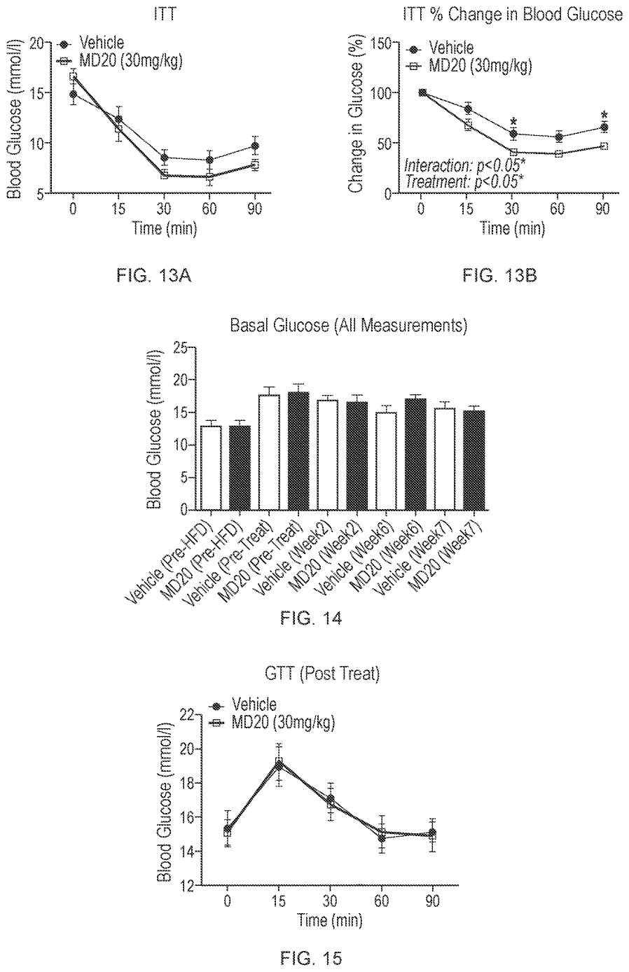

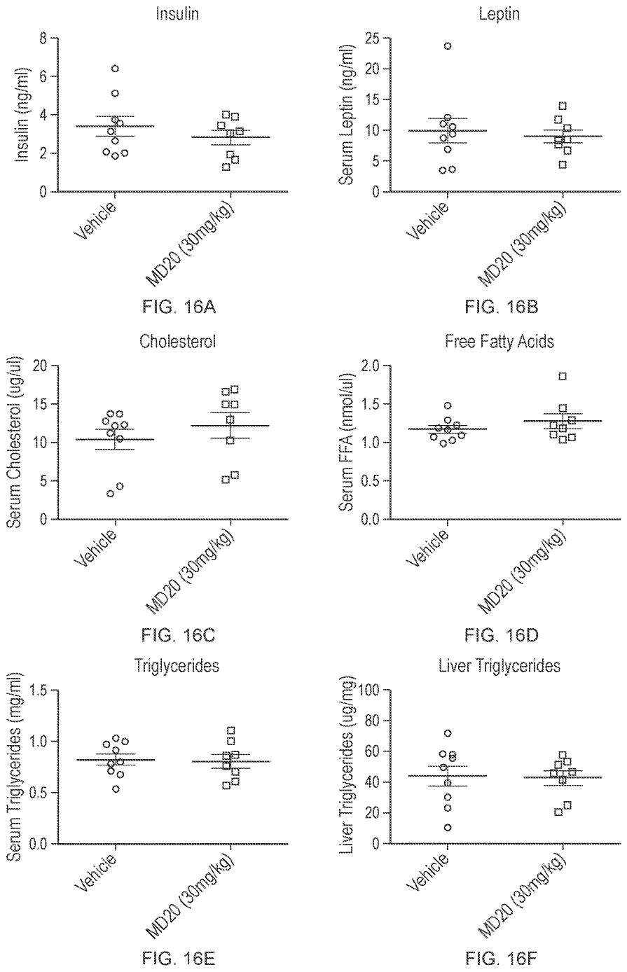

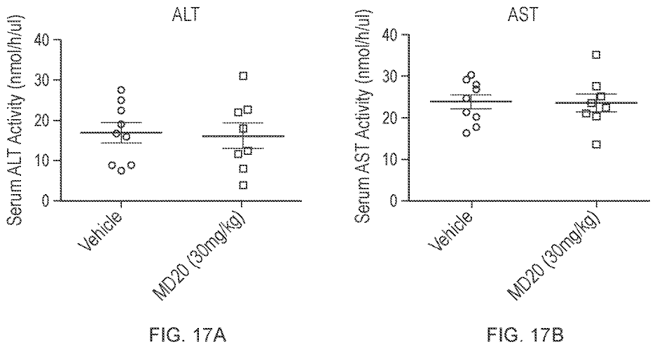

Daily IP administration of MD20 (30 mg/kg) inhibited body weight gain in HFD-fed mice as compared to the control treatment (FIGS. 11A-11B). This was associated with inhibited fat mass gain (FIGS. 12A-12C and 12E). MD20 administration improved insulin sensitivity during ITT in HFD-fed mice as compared to vehicle-treated mice (FIGS. 13A-13B). No significant differences between MD20-treated and control mice were detected in fasted basal glucose levels (FIG. 14) or GTT (FIG. 15). No significant differences were detected in fasted serum insulin, leptin, cholesterol, free fatty acids and serum or liver triglyceride levels (FIGS. 16A-16F). No significant differences in serum ALT and AST levels were detected between MD20-treated and control mice (FIGS. 17A-17B), suggesting that seven week administration of MD20 most likely did not induce hepatic toxicity in HFD-fed mice.

Thus, this Example demonstrates that MD20 (30 mg/kg) administration inhibited fat mass gain in HFD mice, as compared to a control treatment. MD20 administration also inhibited body weight gain and improved insulin sensitivity (as assessed by ITT) in HFD mice. This was similar to the effect observed in the toxicity study described in Example 6, in which administration of MD20 (30 mg/kg) to mice on standard mouse diet was found to lower fat mass.

### Example 8. Effects of MD18 and MD20 in a db/db Genetic Mouse Model of Diabetes and Obesity

This example compares the efficacy of the MD18 and MD20 peptides in the db/db genetic mouse model of diabetes and obesity.

The db/db mouse model is a genetic mouse model of severe obesity and type 2 diabetes. The mouse model carries a mutation in the leptin receptor (Leprdb) resulting in non-functioning leptin receptor, causing obesity phenotypes as early as 3-4 weeks of age (Alpers and Hudkins (2011) CURR. OPIN. NEPHROL. HYPERTENS. 20(3): 278; Susztak et al., (2003) DIABETES 53(3): 784-794). This mouse model displays severe hyperglycemia, hyperinsulinemia, and hyperleptinemia.

Although the db/db mouse model is a widely used model for obesity and type 2 diabetes research, because db/db mouse phenotypes are very severe, it may have limited applicability when studying phenotypes of early stages of obesity and type 2 diabetes or prevention of these conditions. Furthermore, although this mouse model displays advanced obesity and type 2 diabetes phenotypes, these are considered to be secondary to the genetic mutations and may not represent disease etiology or progression in humans given that leptin or leptin receptor deficiency is typically not a common contributor to Type 2 diabetes in humans (Wang et al. (2014) CURRENT DIABETES REVIEWS; 10(2):131-45). Nevertheless, the db/db mouse model was sufficient to demonstrate the efficacy of the peptide MD18 and MD20.

### Materials and Methods

Animals: All animals were housed and tested following European (Directive on the Protection of Animals used for Scientific Purposes, 2010/63/EU) and UK Home Office regulations. Experiments were approved by the University Ethics Board and carried out in accordance with the Animal (Scientific Procedures) Act 1986 and following ARRIVE guidelines. Db/db male mice (~4 weeks old) were purchased from Jackson, USA. All mice received standard mouse chow diet and had ad libitum access to food and water and were kept in a 12 h light/dark cycle.

Treatments: Mice were assigned and counterbalanced according to their body weights in to the following groups: control PBS (n=10); MD18 at 15 mg/kg (n=7), 30 mg/kg (n=8), or 45 mg/kg (n=7); or MD20 at 15 mg/kg (n=9), 30 mg/kg (n=10) or 45 mg/kg (n=7). MD18 and MD20 were administered once daily via subcutaneous (SC) injection for 41 days, whereas control mice received a daily SC injection of vehicle (PBS). MD18 and MD20 were kept in −20 °C and dissolved in sterile phosphate buffered saline (PBS).

Body weights and body mass composition: Body weights were recorded twice weekly during the study, and fat mass and lean mass composition was analysed using an EchoMRI scanner (EchoMedical Systems, Houston, TX, U.S.A.).

Metabolic measurements: All animals were five-hour fasted before obtaining basal glucose measurements and GTT, ITT, and corresponding treatments were administered 1 hour before glucose measurements. Tail blood glucose was measured using AlphaTRAK 2 glucose strips and glucometer (Abbot, UK), or blood was collected and glucose assay (AB65333, Abcam) was performed where readings were too high to be recorded via glucometer. Human Actrapid Insulin (NovaNordisk) at 1 IU/kg dose was administered during ITT.

Serum and liver assays: Trunk-derived blood was collected from 5 h-fasted mice into serum separator microtubes and kept at −80 °C. Serum was used for insulin (#EZRMI-13K, Merck) and leptin (#90030, Crystal Chem) analyses.

Statistical analyses: Data were reported as means±SEM and analysed using one- or two-way ANOVA with Bonferroni post hoc tests, as appropriate. GraphPad Prism (V8, GraphPad Software, Inc., San Diego, California, USA) was used for statistical analyses. P-values <0.05 were considered significant.

### Results and Discussion

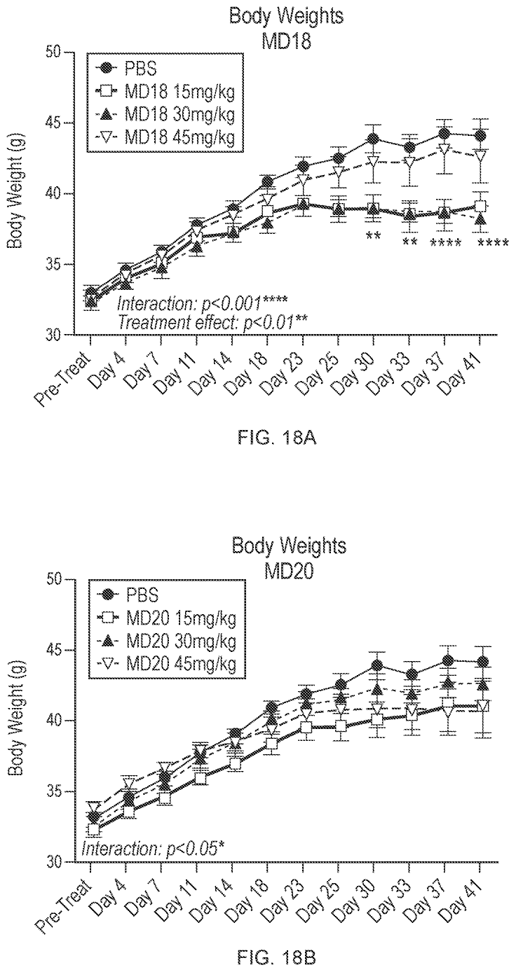

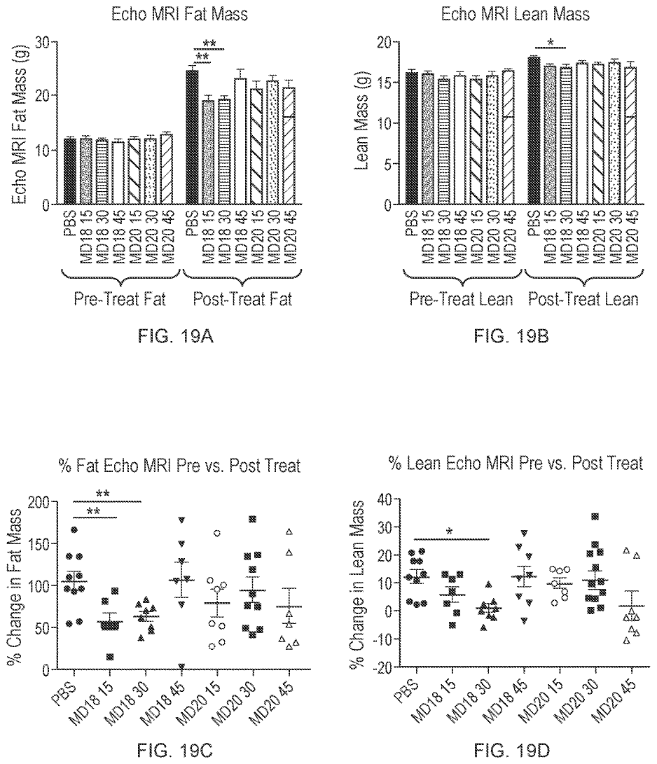

Daily SC administration of MD18 at 15 mg/kg and 30 mg/kg doses significantly inhibited body weight gain in db/db mice as compared to the control treatment (FIG. 18A).

Daily administration of MD20 also inhibited body weight gain in db/db mice, as indicated by a significant overall interaction, although the effects were less prominent as compared to treatment with MD18 (FIG. 18B). Daily SC administration of MD18 at 15 mg/kg and 30 mg/kg doses also inhibited fat mass gain (FIGS. 19A-19D).

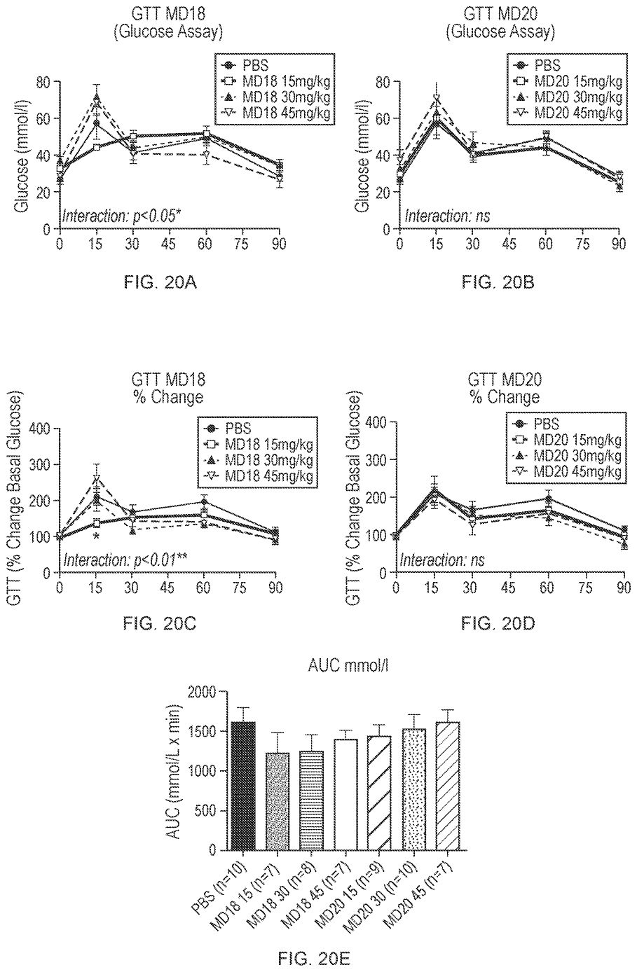

A glucose tolerance test (GTT) performed at week 4 of treatment demonstrated that mice treated with daily SC MD18 at 15 mg/kg displayed better glucose clearance at 15-minutes post-glucose injection, as compared to control mice at the same timepoint (FIGS. 20A, 20C, 20E). No significant differences in glucose clearance during a GTT test were detected in MD20-treated mice as compared to control mice (FIGS. 20B, 20D, 20E).

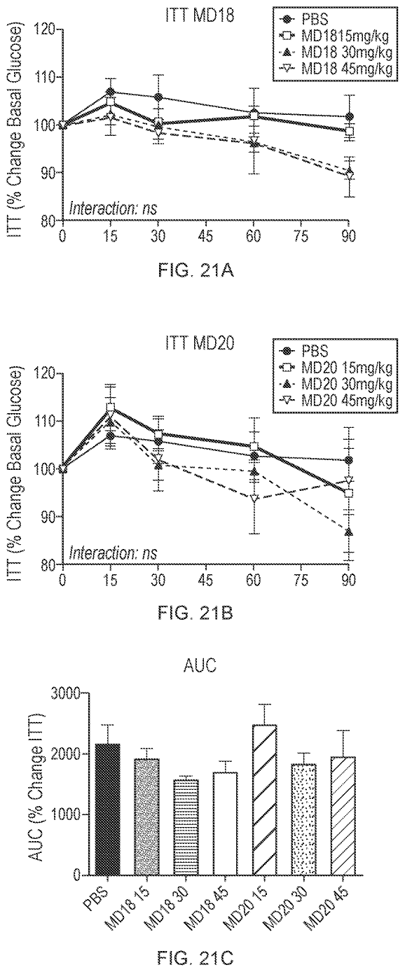

An insulin tolerance test (ITT) performed at week 6 of treatment did not detect any significant differences between the MD18 and MD20 treatment groups and the control mice, although visual trends of improved insulin sensitivity could be seen in this extreme insulin-resistant mouse model (FIGS. 21A-21C).

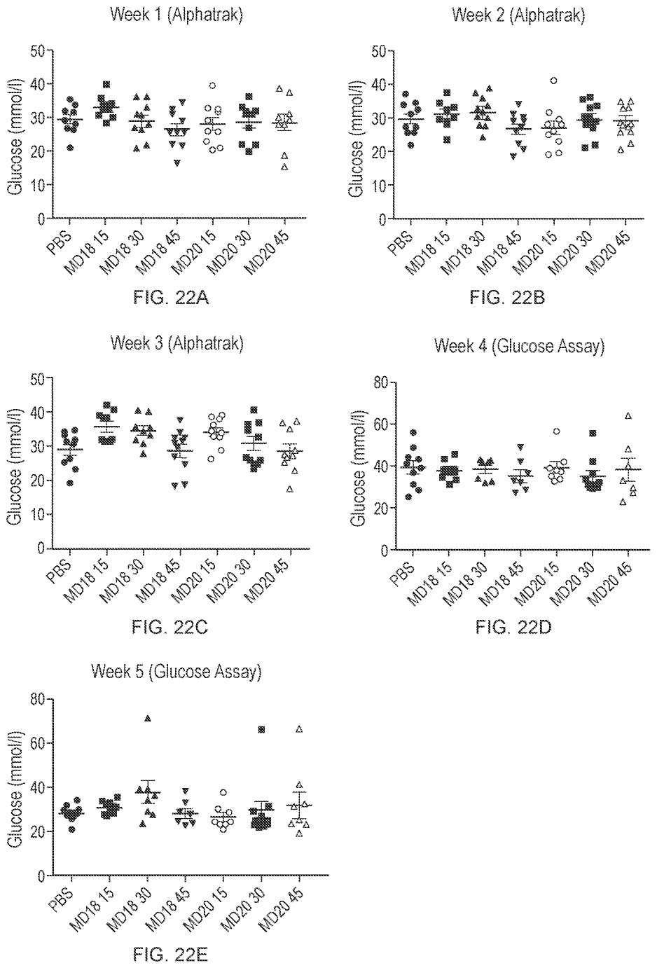

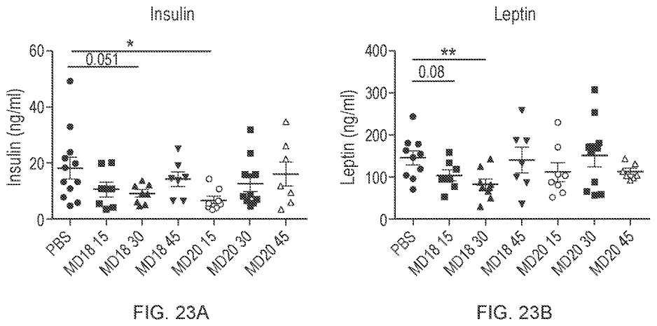

No significant differences in 5-hour-fasted basal glucose levels were detected between MID18-treated or MD20-treated db/db mice, as compared to vehicle-treated db/db mice (FIGS. 22A-22E). A trend for lower fasted insulin levels was detected in the MD18 30 mg/kg treatment group as compared to control mice, and a significant decrease in insulin levels was detected in the MD20 15 mg/kg treatment group as compared to control mice (FIG. 23A). A trend for decreased leptin levels was detected in the MD18 15 mg/kg treatment group as compared to control mice, and a significant decrease in leptin levels was detected in the MD20 30 mg/kg treatment group as compared to control mice (FIG. 23B).

In summary, the results of this study in a genetic mouse model of severe obesity and diabetes (db/db) demonstrated that MD18 treatment had better efficacy as compared to MD20 treatment with respect to inhibiting body weight, inhibiting fat mass gain, and improving glucose clearance during GTT.

### Example 9. Effects of MD20 in a Mouse Model of Atherosclerosis (LDLR^{-/-})

This example demonstrates the efficacy of the MD20 peptide in a mouse mode of atherosclerosis.

Type 2 diabetes and atherosclerosis are connected through various pathological pathways (Poznyak et al. (2020) INT. J. MOL. SCI. 21(5): 1835). Evidence has shown that type 2 diabetes accelerates the risk of developing atherosclerosis, and several mechanisms and risk factors (e.g. obesity, dyslipidemia, insulin resistance) are shared between the two disorders (Watson et al. (2003) J. CARDIOVASC. PHARMACOL. 8(4): 253-260). Around 75% of mortality in type 2 diabetes patients is also linked to causes associated with atherosclerosis (Hurst and Lee (2003) ANN. INTERN. MED. 139(10): 824-834). Hence, MD20 treatment was tested for efficacy in a widely-studied LDLR−/− mouse model of atherosclerosis. The LDLR−/− mouse model lacks functional low density lipoprotein (LDL) receptor (LDLR), which impairs the clearance of low-density lipoproteins from circulation and results in increased levels of cholesterol, leading to formation of atherosclerotic plaques upon HFD-feeding.

### Materials and Methods

Animals: All animals were housed and tested following European (Directive on the Protection of Animals used for Scientific Purposes, 2010/63/EU) and UK Home Office regulations. Experiments were approved by the University Ethics Board and carried out in accordance with the Animal (Scientific Procedures) Act 1986 and following ARRIVE guidelines. LDLR−/− male mice (~6-7 weeks old) were purchased from Jackson, US. All mice received a high fat/high cholesterol diet (42% fat plus 0.2% cholesterol, #TD.88137, Envigo, US), to induce atherosclerosis phenotype associated with obesity and type 2 diabetes. All mice had ad libitum access to food and water and were kept in a 12 h light/dark cycle.

Treatments: After 8 weeks of high fat/high cholesterol diet feeding, mice were randomized in to control (n=10) or MD20 30 mg/kg (n=9) groups. MD20 compound (30 mg/kg) was administered once daily via intraperitoneal (IP) injection for 7 weeks, whereas control mice received a daily IP injection of vehicle (PBS). MD20 was kept at −20 °C and dissolved in sterile phosphate buffered saline (PBS) freshly every day before the injection. Two mice died during the study and final numbers per group were: control (n=8) and MD20 30 mg/kg (n=9).

Bodyweights and body mass composition: Body weights were recorded during the study, and fat and lean mass composition was analyzed using an EchoMRI scanner (EchoMedical Systems, Houston, TX, U.S.A.).

Metabolic measurements: All animals were 5 h-fasted before metabolic tests. Tail blood glucose was measured using AlphaTRAK 2 glucose strips and glucometer (Abbot, UK).

Glucose and insulin tolerance tests (GTT, ITT) were performed by IP injection of 2 mg/g of glucose (GTT) or 0.6 mU/g insulin (ITT), and glucose levels were measured at 15, 30, 60 and 90 min post-injection.

Serum and liver assays: Trunk-derived blood was collected from 5 h-fasted mice into serum separator microtubes and kept at −80 °C. Serum was used for insulin (#EZRMI-13K, Merck), leptin (#90030, Crystal Chem), free fatty acids (FFA; #mak044, Sigma-Aldrich), cholesterol (#MAK043, Sigma Aldrich), and alanine and aspartate aminotransferase (ALT and AST; ab105134 and ab105135, Abcam) analyses. Serum and liver triglycerides were determined using triglyceride reagents (#T2449, #F6428, #G7793, Sigma Aldrich).

Aortic plaques analyses: Hearts with aortas attached were immersed in formalin and stored at 4 °C for 24 h following dissection, and transferred in to PBS thereafter. Aortas were sub-dissected under microscope and prepared for staining with Oil Red O. Images were captured using a light microscope. Plaque formation was quantified using ImageJ software, and results were expressed as % plaque area.

Gene expression analyses: Livers and adipose tissue were collected from 5 h-fasted animals and snap-frozen in liquid nitrogen. RNA was isolated from frozen liver tissue using TRIzole (Sigma, UK) and cDNA was synthesized from 1 μg RNA using bioscript cDNA synthesis kit (Bioline, UK), oligo (dT) 18 (SEQ ID NO: 170), and random hexamer primers.

Genes of interest were amplified by quantitative PCR (qPCR) using gene-specific primers and GoTaq qPCR master mix (Promega, Madison, WI, USA).

Statistical analyses: Data were reported as means±SEM and analyzed using one- or two-way ANOVA with Bonferroni post hoc tests, as appropriate. GraphPad Prism (V8, GraphPad Software, Inc., San Diego, California, USA) was used for statistical analyses. P-values <0.05 were considered significant.

### Results and Discussion

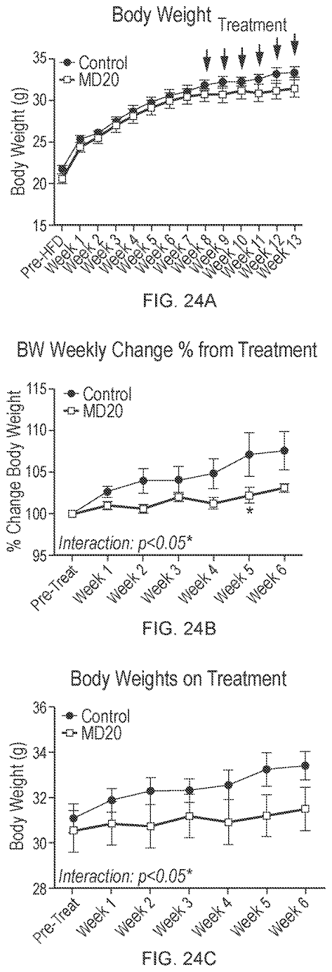

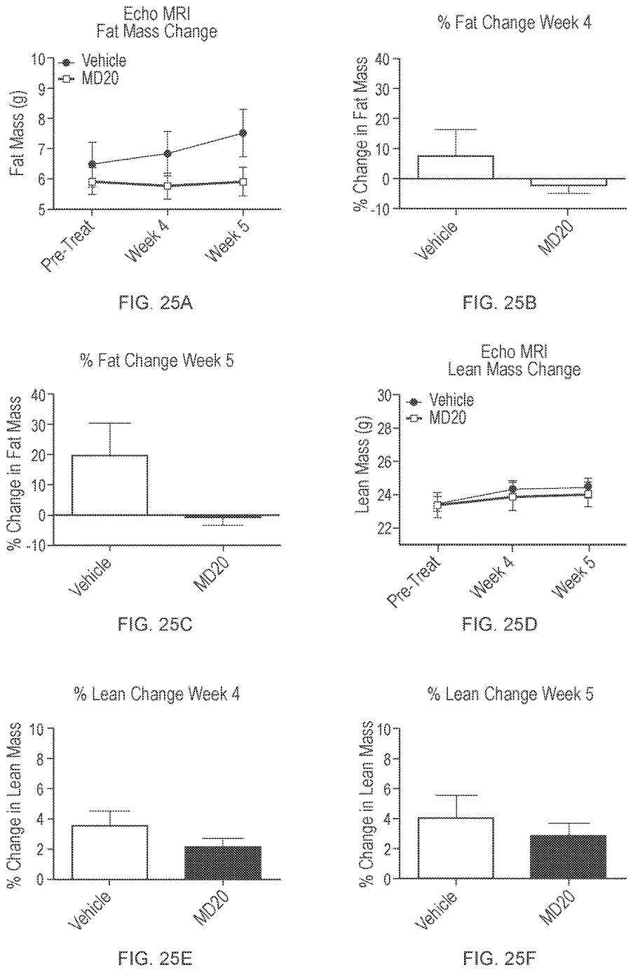

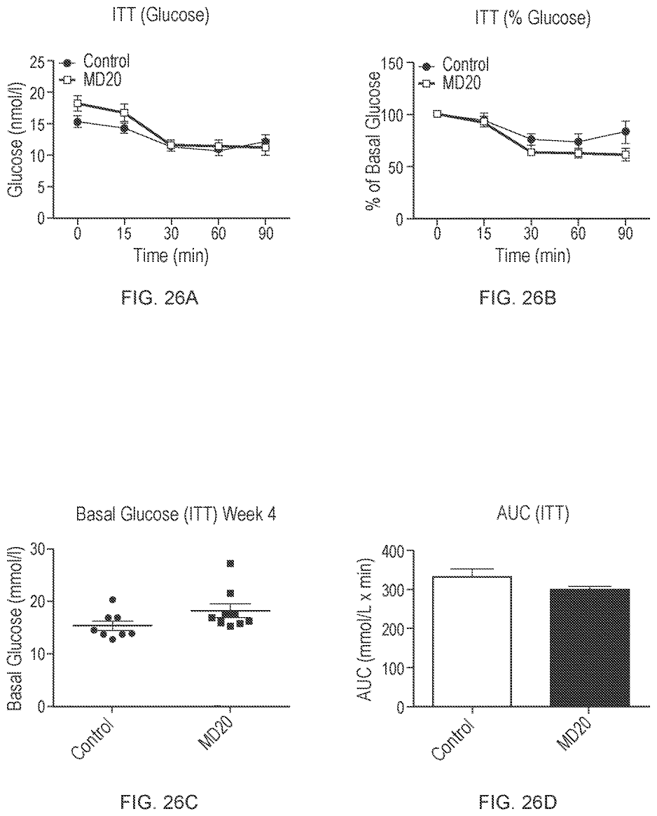

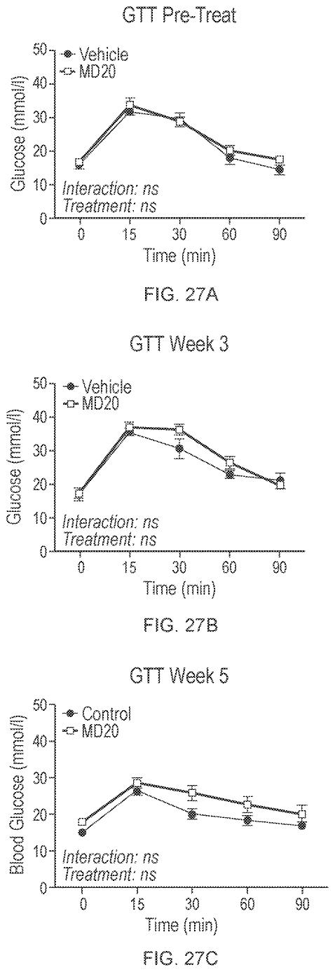

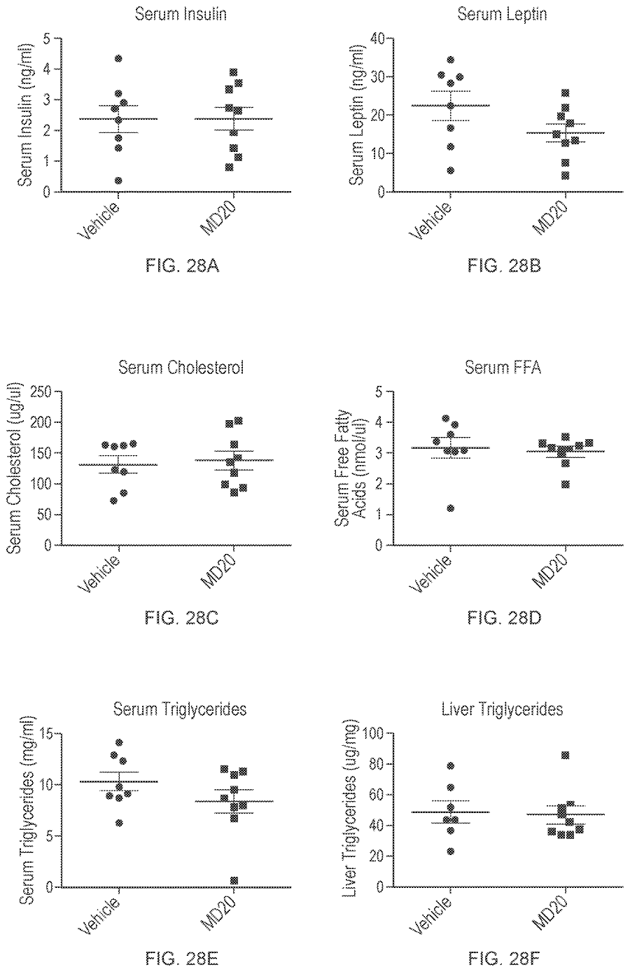

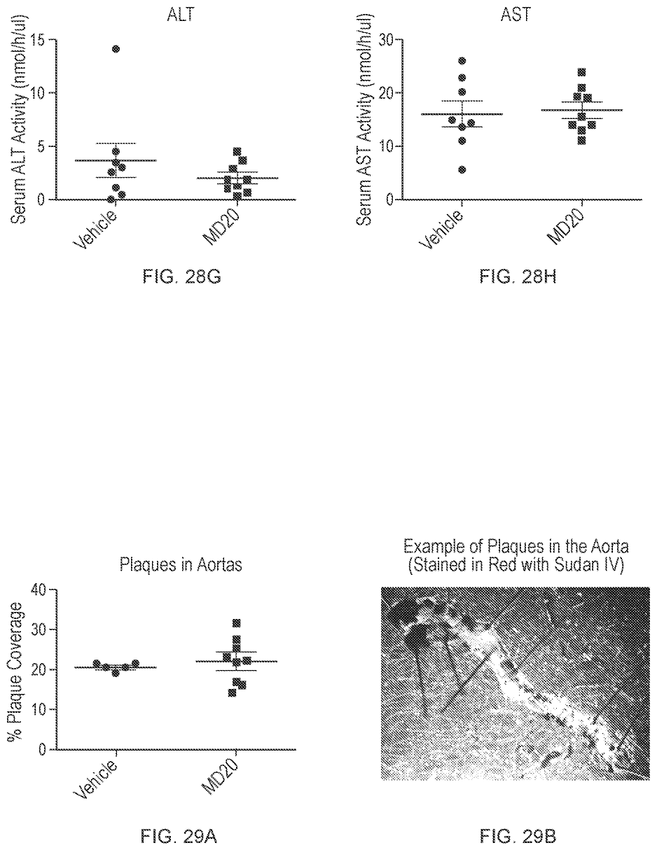

No statistically significant differences in raw body weights between MD20-treated and vehicle-treated LDLR−/− mice were detected (FIG. 24A). However, a significant interaction was found in % change in body weights between vehicle and MD20-treated mice (FIG. 24B) and in body weights during the weeks of treatment (FIG. 24C). No statistically significant differences were detected in fat mass content between MD20-treated and vehicle-treated LDLR−/− mice, although a trend to ward a decreased % change in fat mass could be seen in MD20-treated mice as compared to control mice (FIGS. 25A-25C). No significant changes in lean mass were found between the groups (FIGS. 25D-25F). Although differences in ITT between MD20-treated and vehicle mice were not statistically significant, slight improvements could be seen in MD20-treated mice (FIGS. 26A-26D). No statistically significant differences were found during GTT between MD20-treated and control mice (FIGS. 27A-27C). No statistically significant differences were found between the groups with respect to 5 h-fasted levels of serum insulin, serum leptin, serum cholesterol, serum free fatty acids, serum triglycerides, or hepatic triglycerides (FIGS. 28A-28F). No statistically significant differences were found between groups with respect to serum ALT and AST levels in 5 h-fasted mice (FIGS. 28G-28H). No statistically significant differences were found in aortic plaque area between MD20-treated and control LDLR−/− mice (FIG. 29A).

Thus, this Example demonstrates that MD20 treatment in LDLR−/− mice resulted in similar effects on body weight and fat mass as were observed in the HFD-induced mouse model (Example 7), where MD20 treatment similarly prevented gains in body weight and fat mass. Hence, this Example confirms that MD20 treatment prevents body weight and fat mass gain upon high fat/high cholesterol feeding in mice.

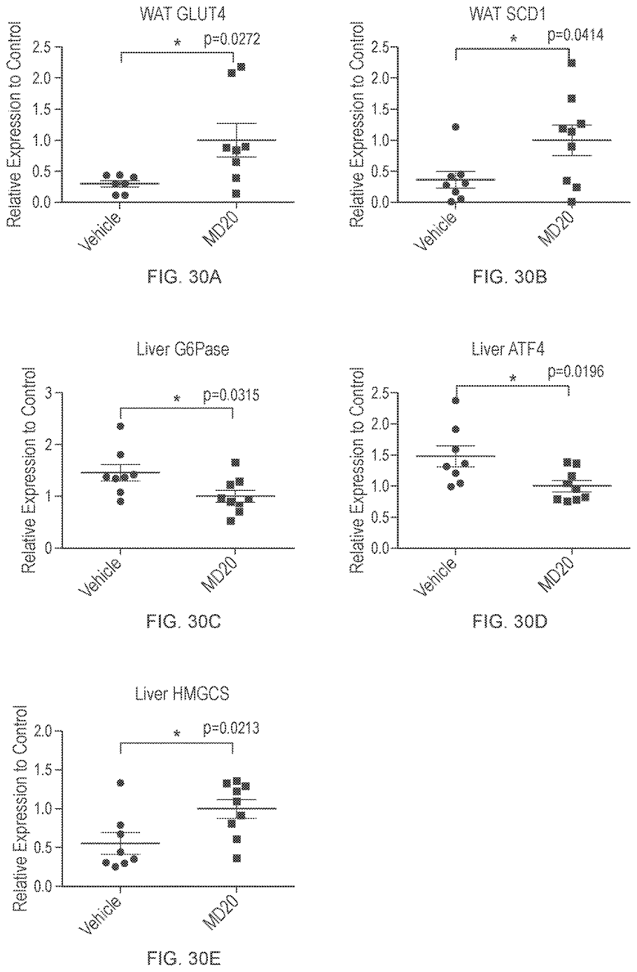

Interestingly, GLUT4 gene expression was found to be increased in the adipose tissue of MD20-treated LDLR −/− mice, as compared to control mice (FIG. 30A). GLUT4 is the key glucose transporter in to adipose tissue, as it mediates insulin-stimulated glucose uptake in adipocytes and muscle by rapidly moving from intracellular storage sites to the plasma membrane (Abel et al. (2001) NATURE 409(6821): 729-733). In insulin-resistant states such as obesity and type 2 diabetes, GLUT4 expression is decreased in adipose tissue. Therefore, increased GLUT4 expression could be a positive effect of M1D20 administration. SCD1 is involved in conversion of saturated fatty acids in to monosaturated fatty acids and adipogenesis, and its expression was also increased in adipose tissue of MD20-treated mice (FIG. 30B), which could be a compensatory mechanism in response to inhibited fat mass gain. Furthermore, expression of G6Pase gene (a key molecule in gluconeogenesis pathway) and ATF4 was decreased in liver tissue of MD20-treated mice (FIGS. 30C-30D), which could imply that MD20 treatment affected the hepatic gluconeogenesis pathway. Expression of HMGCS was also upregulated in MD20-treated mice (FIG. 30E).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

| SEQ | |
|---|---|
| ID NO | Sequence |
| 1 | PX₁PPX₂R |
| | wherein X₁ and X₂ are each independently |
| | any amino acid. |
| 2 | PX1X2PX3R |
| | wherein X₁, X₂, and X₃ are each |
| | independently any amino acid. |
| 3 | PX₁X₂PX₃RX₄ |
| | wherein X₁, X₂, X₃, and X₄ are each |
| | independently any amino acid. |
| 4 | PRPPKRX₁ |
| | wherein X₁ is any amino acid. |
| 5 | PRPPKRI |
| 6 | PRPPKRIL |
| 7 | PRPPKRILE |
| 8 | PRPPKR |
| 9 | PPRPPKR |
| 10 | PPPEHIPPPPRPPKR |
| 11 | PPPRPPKR |
| 12 | PPPPRPPKR |
| 13 | IPPPPRPPKR |
| 14 | HIPPPPRPPKR |
| 15 | EHIPPPPRPPKR |
| 16 | PEHIPPPPRPPKR |
| 17 | PPEHIPPPPRPPKR |
| 18 | PPRPPKRI |
| 19 | PPRPPKRIL |
| 20 | PPRPPKRILE |
| 21 | PPPEHIPPPPRPPKRILE |
| 22 | APPEHIPPPPRPPKR |
| 23 | PAPEHIPPPPRPPKR |
| 24 | PPAEHIPPPPRPPKR |
| 25 | PPPAHIPPPPRPPKR |
| 26 | PPPEAIPPPPRPPKR |
| 27 | PPPEHAPPPPRPPKR |
| 28 | PPPEHIAPPPRPPKR |
| 29 | PPPEHIPAPPRPPKR |
| 30 | PPPEHIPPAPRPPKR |
| 31 | PPPEHIPPPARPPKR |
| 32 | PPPEHIPPPPAPPKR |
| 33 | PPPEHIPPPPRAPKR |
| 34 | PPPEHIPPPPRPAKR |
| 35 | PPPEHIPPPPRPPAR |
| 36 | PPPEHIPPPPRPPKA |
| 37 | ARPPKR |
| 38 | PAPPKR |
| 39 | PRAPKR |
| 40 | PRPAKR |
| 41 | PRPPAR |
| 42 | PRPPKA |
| 43 | PKR |
| 44 | PPK |
| 45 | RPPK |
| 46 | PPKR |
| 47 | RPPKR |
| 48 | PRP |
| 49 | RPP |
| 50 | PRPP |
| 51 | PRPPK |
| 52 | PPRPPK |
| 53 | PPPRPP |
| 54 | PPPPRP |
| 55 | IPPPPR |
| 56 | EHIPPP |
| 57 | PPEHIP |
| 58 | PPPRPPK |
| 59 | APRPPKR |
| 60 | PPRPPKK |
| 61 | RPRPPKR |
| 62 | NPRPPKR |
| 63 | CPRPPKR |
| 64 | EPRPPKR |
| 65 | HPRPPKR |
| 66 | FPRPPKR |
| 67 | SPRPPKR |
| 68 | PPKPPKR |
| 69 | PPFPPKR |
| 70 | PPHPPKR |
| 71 | PPEPPKR |
| 72 | PPSPPKR |
| 73 | PPNPPKR |
| 74 | PPCPPKR |
| 75 | PPRRPKR |
| 76 | PPRFPKR |
| 77 | PPRPPRR |
| 78 | PPRPPHR |
| 79 | PPRPPWR |
| 80 | PPRPPER |
| 81 | PPRPPSR |
| 82 | PPRPPNR |
| 83 | PPRPPCR |
| 84 | PPEHIPPP |
| 85 | SLSR |
| 86 | PPRGPKR |
| 87 | PPRIPKR |
| 88 | PPRLPKR |
| 89 | PPRKPKR |
| 90 | PPRSPKR |
| 91 | PPRNPKR |
| 92 | PPRDPKR |
| 93 | PPRCPKR |
| 94 | PPREPKR |
| 95 | PPRQPKR |
| 96 | PPRHPKR |
| 97 | PPRMPKR |
| 98 | PPRTPKR |
| 99 | PPRWPKR |
| 100 | PPRYPKR |
| 101 | PPRVPKR |
| 102 | PPRPPGR |
| 103 | PPRPPIR |
| 104 | PPRPPLR |
| 105 | PPRPPFR |
| 106 | PPRPPDR |
| 107 | PPRPPMR |
| 108 | PPRPPQR |
| 109 | PPRPPPR |
| 110 | PPRPPTR |
| 111 | PPRPPYR |
| 112 | PPRPPVR |
| 113 | PPDPPKR |
| 114 | PPQPPKR |
| 115 | PPGPPKR |
| 116 | PPLPPKR |
| 117 | PPIPPKR |
| 118 | PPPPPKR |
| 119 | PPMPPKR |
| 120 | PPTPPKR |
| 121 | PPWPPKR |
| 122 | PPYPPKR |
| 123 | PPVPPKR |
| 124 | DPRPPKR |
| 125 | QPRPPKR |
| 126 | GPRPPKR |
| 127 | IPRPPKR |
| 128 | LPRPPKR |
| 129 | KPRPPKR |
| 130 | MPRPPKR |
| 131 | TPRPPKR |
| 132 | WPRPPKR |
| 133 | YPRPPKR |
| 134 | VPRPPKR |
| 135 | PPPEHIPPPPRPPKRILEPH |
| 136 | HEDLEPPPEHIPPPPRPPKR |
| 137 | PPPEHIPPPPRPPKRILEPHNGKCR |
| 138 | PAVPPRP |
| 139 | SPAKGEPSLPEK |
| 140 | MAEEVVVVAKFDYVAQQEQELDIKKNERLWLLDDSKSWWRVRN |
| | SMNKTGFVPSNYVERKNSARKASIVKNLKDTLGIGKVKRKPSV |
| | PDSASPADDSFVDPGERLYDLNMPAYVKFNYMAEREDELSLIK |
| | GTKVIVMEKCSDGWWRGSYNGQVGWFPSNYVTEEGDSPLGDHV |
| | GSLSEKLAAVVNNLNTGQVLHVVQALYPFSSSNDEELNFEKGD |
| | VMDVIEKPENDPEWWKCRKINGMVGLVPKNYVTVMQNNPLTSG |
| | LEPSPPQCDYIRPSLTGKFAGNPWYYGKVTRHQAEMALNERGH |
| | EGDFLIRDSESSPNDFSVSLKAQGKNKHFKVQLKETVYCIGQR |
| | KFSTMEELVEHYKKAPIFTSEQGEKLYLVKHLS |
| 141 | PPPEHIPPPPRPPKRILEPHNGKCREFFPN |
| 142 | WKELSHEDLEPPPEHIPPPPRPPKR |
| 143 | SVQDQWKELSHEDLEPPPEHIPPPPRPPKR |
| 144 | PPRPPKRA |
| 145 | PPRPPKRR |
| 146 | PPRPPKRN |
| 147 | PPRPPKRD |
| 148 | PPRPPKRC |
| 149 | PPRPPKRF |
| 150 | PPRPPKRS |
| 151 | PPRPPKRG |
| 152 | PPRPPKRH |
| 153 | PPRPPKRL |
| 154 | PPRPPKRK |
| 155 | PPRPPKRM |
| 156 | PPRPPKRP |
| 157 | PPRPPKRW |
| 158 | PPRPPKRY |
| 159 | acctgtacttccagggcatggagatggaaaaggagttc |
| 160 | gtgcaggaggagttcgaattctatgtgttgctgttgaac |
| 161 | gtgcaggaggagttcgaattTCATTTCCCATTGTGTGG |
| 162 | gtgcaggaggagttcgaatttcagtctttatcctcctg |
| 163 | gtgcaggaggagttcgaattttaggcaccttcgatcac |
| 164 | acctgtacttccagggcatgatgcccgcttatgtgaaatttaa |
| | c |
| 165 | gtgcaggaggagttcgaattctagtcaccttcttcagttacat |
| | ag |
| 166 | TGTCGATCCTACCATCCA |
| 167 | AGTCAGTCAGTGCAGGAG |
| 168 | H-Pyr-PRPPKR |
| 169 | H-Pyr-RPPKR |
| 170 | TTTTTTTTTTTTTTTTTT |

What is claimed is:

1. A synthetic peptide consisting essentially of an amino acid sequence of six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or six to fifty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 1
(SEQ ID NO: 1)
PX₁PPX₂R
wherein X₁ and X₂ are each independently any amino acid, and
(a) the proline at position 1 of SEQ ID NO: 1 is the N-terminal amino acid and is optionally modified by covalent linkage to a carbonyl-containing moiety;
(b) the N-terminal amino acid is chemically modified (e.g., acetylated) and/or the C-terminal amino acid is chemically modified (e.g., amidated), or
(c) the C-terminal amino acid is arginine provided that, (i) if X₂ is arginine, then X₁ is not arginine or glutamine, (ii) if X₂ is serine, X₁ is not arginine, and (iii) if X₂ is methionine, then X₁ is not a histidine.

2. The synthetic peptide of claim 1, consisting essentially of an amino acid sequence of six to thirty consecutive amino acids.

3. The synthetic peptide of claim 1, consisting essentially of an amino acid sequence of six to fifty consecutive amino acids.

4. A synthetic peptide consisting essentially of an amino acid sequence of six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or six to fifty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 1
(SEQ ID NO: 1)
PX₁PPX₂R
wherein X₁ and X₂ are each independently any amino acid, and
(a) the proline at position 1 of SEQ ID NO: 1 is the N-terminal amino acid and is optionally modified by covalent linkage to a carbonyl-containing moiety; or
(b) the N-terminal amino acid is acetylated and the C-terminal amino acid is amidated.

5. The synthetic peptide of claim 4, consisting essentially of an amino acid sequence of six to thirty consecutive amino acids.

6. The synthetic peptide of claim 4, consisting essentially of an amino acid sequence of six to fifty consecutive amino acids.

7. The synthetic peptide of any one of claims 1-6, wherein X₁ is a charged amino acid.

8. The synthetic peptide of claim 7, wherein X₁ is a positively charged amino acid.

9. The synthetic peptide of claim 8, wherein X₁ is selected from the group consisting of arginine, histidine, and lysine.

10. The synthetic peptide of claim 7, wherein X₁ is a negatively charged amino acid.

11. The synthetic peptide of claim 10, wherein X₁ is glutamic acid.

12. The synthetic peptide of claim any one of claims 1-6, wherein X₁ is an uncharged amino acid.

13. The synthetic peptide of claim 12, wherein X₁ is a hydrophobic amino acid.

14. The synthetic peptide of claim 12, wherein X₁ is selected from the group consisting of alanine, asparagine, and serine.

15. The synthetic peptide of any one of claims 1-14, wherein X₂ is a charged amino acid.

16. The synthetic peptide of claim 15, wherein X₂ is a positively charged amino acid.

17. The synthetic peptide of claim 16, wherein X₂ is arginine, histidine, or lysine.

18. The synthetic peptide of claim 17, wherein X₂ is a negatively charged amino acid.

19. The synthetic peptide of claim 18, wherein X₂ is glutamic acid.

20. The synthetic peptide of any one of claims 1-14, wherein X₂ is an uncharged amino acid.

21. The synthetic peptide of claim 20, wherein X₂ is selected from the group consisting of alanine, asparagine, cysteine, serine, and tryptophan.

22. The synthetic peptide of claim 20, wherein X₂ is a hydrophobic amino acid.

23. The synthetic peptide of claim 22, wherein X₂ is selected from the group consisting of alanine, cysteine, and tryptophan.

24. The synthetic peptide of claim any one of claims 1-6, wherein X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine.

25. The synthetic peptide of claim 24, wherein X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine.

26. The synthetic peptide of claim 25, wherein X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

27. The synthetic peptide of any one of claims 1-6, or 24-26, wherein X₂ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

28. The synthetic peptide of claim 27, wherein X₂ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine.

29. The synthetic peptide of claim 28, wherein X₂ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

30. A synthetic peptide consisting essentially of an amino acid sequence of seven to ten consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid;
X₂ is a non-aromatic amino acid;
if X₁ is alanine and X₂ is valine, then X₃ is not proline;
if X₂ is proline and X₃ is arginine, then X₁ is not arginine or glutamine; and
if X₂ is proline and X₃ is methionine, then X₁ is not histidine.

31. A synthetic peptide consisting essentially of an amino acid sequence of eleven consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid;
X₄ is either absent or any amino acid; and
if X₂ is proline and X₄ is not absent, then
(i) X₁ is not threonine;
(ii) X₃ is not lysine; and/or
(iii) X₄ is not serine.

32. A synthetic peptide consisting essentially of an amino acid sequence of twelve consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid;
X₄ is either absent or any amino acid;
if X₂ is proline and X₄ is not absent, then
(i) X₁ is not arginine;
(ii) X₃ is not threonine; and/or
(iii) X₄ is not threonine.

33. A synthetic peptide consisting essentially of an amino acid sequence of thirteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid; and
X₄ is either absent or any amino acid.

34. A synthetic peptide consisting essentially of an amino acid sequence of fourteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid;
X₄ is either absent or any amino acid;
if X₁ is alanine and X₂ is valine, then X₃ is not proline; and
if X₂ is proline and X₄ is absent, then:
(i) X₁ is not arginine; and/or
(ii) X₃ is not serine.

35. A synthetic peptide consisting essentially of an amino acid sequence of fifteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid;
X₄ is either absent or any amino acid; and
if X₂ is proline and X₄ is not absent, then:
(i) if X₁ is arginine, X₃ is lysine, and X₄ is isoleucine, then X₄ is not the C-terminal amino acid;
(ii) if X₄ is alanine, then X₁ is not proline and/or X₃ is not arginine; and
(iii) if X₄ is proline, then:
(a) if X₁ is methionine, then X₃ is not asparagine; and
(b) if X₁ is arginine, then X₃ is not glycine.

36. A synthetic peptide consisting essentially of an amino acid sequence of fifteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid;
X₄ is either absent or any amino acid; and
if X₂ is proline and X₄ is not absent, then:
(i) if X₁ is arginine, X₃ is lysine, and X₄ is isoleucine, then X₄ is not the C-terminal amino acid and/or the N-terminal amino acid is not a proline;
(ii) if X₄ is alanine, then X₁ is not proline and/or X₃ is not arginine; and
(iii) if X₄ is proline, then:
(a) if X₁ is methionine, then X₃ is not asparagine; and
(b) if X₁ is arginine, then X₃ is not glycine.

37. A synthetic peptide consisting essentially of an amino acid sequence of (i) sixteen, seventeen, eighteen, nineteen, twenty, twenty-one, twenty-two, twenty-three, twenty-four, twenty-five, twenty-six, twenty-seven, twenty-eight, twenty-nine, or thirty consecutive amino acids, or (ii) sixteen, seventeen, eighteen, nineteen, twenty, twenty-one, twenty-two, twenty-three, twenty-four, twenty-five, twenty-six, twenty-seven, twenty-eight, twenty-nine, thirty, thirty-one, thirty-two, thirty-three, thirty-four, thirty-five, thirty-six, thirty-seven, thirty-eight, thirty-nine, forty, forty-one, forty-two, forty-three, forty-four, forty-five, forty-six, forty-seven, forty-eight, forty-nine, or fifty consecutive amino acids, each including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is a non-aromatic amino acid;
X₄ is either absent or any amino acid;
wherein, if the synthetic peptide consists essentially of twenty consecutive amino acids, then the four most C-terminal amino acid residues of the synthetic peptide is not SLSR (SEQ ID NO: 85).

38. The synthetic peptide of claim 37, consisting essentially of an amino acid sequence of sixteen to thirty consecutive amino acids.

39. The synthetic peptide of claim 37, consisting essentially of an amino acid sequence of sixteen to fifty consecutive amino acids.

40. The synthetic peptide of any one of claims 32-39, wherein X₁ is a charged amino acid.

41. The synthetic peptide of claim 40, wherein X₁ is a positively charged amino acid.

42. The synthetic peptide of claim 41, wherein X₁ is arginine, histidine, or lysine.

43. The synthetic peptide of claim 40, wherein X₁ is a negatively charged amino acid.

44. The synthetic peptide of claim 43, wherein X₁ is glutamic acid.

45. The synthetic peptide of any one of claims 32-39, wherein X₁ is an uncharged amino acid.

46. The synthetic peptide of claim 45, wherein X₁ is a hydrophobic amino acid.

47. The synthetic peptide of claim 45, wherein X₁ is selected from the group consisting of alanine, asparagine, and serine.

48. The synthetic peptide of any one of claims 32-47, wherein X₂ is an uncharged, non-aromatic amino acid.

49. The synthetic peptide of claim 48, wherein X₂ is alanine or proline.

50. The synthetic peptide of claim 48, wherein X₂ is a hydrophobic, non-aromatic amino acid.

51. The synthetic peptide of claim 50, wherein X₂ is alanine.

52. The synthetic peptide of any one of claims 32-47, wherein X₂ is a charged amino acid.

53. The synthetic peptide of claim 52, wherein X₂ is a positively charged amino acid.

54. The synthetic peptide of claim 53, wherein X₂ is arginine.

55. The synthetic peptide of any one of claims 32-54, wherein X₃ is a charged amino acid.

56. The synthetic peptide of claim 55, wherein X₃ is a positively charged amino acid.

57. The synthetic peptide of claim 56, wherein X₃ is arginine, histidine, or lysine.

58. The synthetic peptide of claim 55, wherein X₃ is a negatively charged amino acid.

59. The synthetic peptide of claim 58, wherein X₃ is glutamic acid.

60. The synthetic peptide of any one of claims 32-59, wherein X₃ is an uncharged amino acid.

61. The synthetic peptide of claim 60, wherein X₃ is selected from the group consisting of alanine, asparagine, cysteine, serine, and tryptophan.

62. The synthetic peptide of claim 60, wherein X₃ is a hydrophobic amino acid.

63. The synthetic peptide of claim 62, wherein X₃ is selected from the group consisting of alanine, cysteine, and tryptophan.

64. The synthetic peptide of claim 33-63, wherein X₄ is a charged amino acid.

65. The synthetic peptide of claim 64, wherein X₄ is a positively charged amino acid.

66. The synthetic peptide of claim 65, wherein X₄ is selected from the group consisting of arginine, histidine, and lysine.

67. The synthetic peptide of claim 64, wherein X₄ is a negatively charged amino acid.

68. The synthetic peptide of claim 67, wherein X₄ is aspartic acid or glutamic acid.

69. The synthetic peptide of claim 33-63, wherein X₄ is an uncharged amino acid.

70. The synthetic peptide of claim 69, wherein X₄ is alanine, asparagine, cysteine, glycine, isoleucine, leucine, methionine, phenylalanine, proline, serine, threonine, tryptophan, tyrosine, or valine.

71. The synthetic peptide of claim 33-63, wherein X₄ is an aliphatic amino acid.

72. The synthetic peptide of claim 71, wherein X₄ is selected from the group consisting of alanine, glycine, isoleucine, leucine, proline, and valine.

73. The synthetic peptide of claim 72, wherein X₄ is isoleucine.

74. The synthetic peptide of claim 33-63, wherein X₄ is an aromatic amino acid.

75. The synthetic peptide of claim 74, wherein X₄ is phenylalanine, tryptophan, or tyrosine.

76. The synthetic peptide of claim 33-63, wherein X₄ is a sulfur-containing amino acid.

77. The synthetic peptide of claim 76, wherein X₄ is cysteine or methionine.

78. The synthetic peptide of claim 33-63, wherein X₄ is an amino acid with a hydroxyl-containing side chain.

79. The synthetic peptide of claim 78, wherein X₄ is serine or threonine.

80. The synthetic peptide of claim 33-63, wherein X₄ is an acidic amino acid.

81. The synthetic peptide of claim 80, wherein X₄ is aspartic acid or glutamic acid.

82. The synthetic peptide of claim 33-63, wherein X₄ is a basic amino acid.

83. The synthetic peptide of claim 82, wherein X₄ is arginine, histidine, or lysine.

84. The synthetic peptide of claim 33-63, wherein X₄ is an amino acid with an amide-containing sidechain.

85. The synthetic peptide of claim 84, wherein X₄ is asparagine or glutamine.

86. The synthetic peptide of claim any one of claims 32-39, wherein X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine.

87. The synthetic peptide of claim 86, wherein X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine.

88. The synthetic peptide of claim 87, wherein X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

89. The synthetic peptide of any one of claims 32-39 or 86-88, wherein X₂ is selected from the group consisting of alanine, arginine, isoleucine, leucine, lysine, proline, threonine, and valine.

90. The synthetic peptide of claim 89, wherein X₂ is selected from the group consisting of alanine, isoleucine, proline, and valine.

91. The synthetic peptide of claim 90, wherein X₂ is selected from the group consisting of isoleucine, proline, and valine.

92. The synthetic peptide of any one of claims 32-39 or 86-91, wherein X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

93. The synthetic peptide of claim 92, wherein X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine.

94. The synthetic peptide of claim 93, wherein X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

95. A synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 4:
(SEQ ID NO: 4)
PRPPKRX₁,
wherein X₁ is absent or any amino acid, and
if X₁ is isoleucine and the synthetic peptide is a 15-mer, then X₁ is not the C-terminal amino acid.

96. The synthetic peptide of claim 95, consisting essentially of an amino acid sequence of six to thirty consecutive amino acids.

97. The synthetic peptide of claim 95, consisting essentially of an amino acid sequence of six to fifty consecutive amino acids.

98. A synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty), or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 4:
(SEQ ID NO: 4)
PRPPKRX₁,
wherein X₁ is absent or any amino acid, and
if X₁ is isoleucine and the synthetic peptide is a 15-mer, then X₁ is not the C-terminal amino acid and/or proline is not the N-terminal amino acid.

99. The synthetic peptide of claim 98, consisting essentially of an amino acid sequence of six to thirty consecutive amino acids.

100. The synthetic peptide of claim 98, consisting essentially of an amino acid sequence of six to fifty consecutive amino acids.

101. The synthetic peptide of any one of claims 95-100, wherein X₁ is a charged amino acid.

102. The synthetic peptide of claim 101, wherein X₁ is a positively charged amino acid.

103. The synthetic peptide of claim 102, wherein X₁ is selected from the group consisting of arginine, histidine, and lysine.

104. The synthetic peptide of claim 101, wherein X₁ is a negatively charged amino acid.

105. The synthetic peptide of claim 104, wherein X₁ is aspartic acid or glutamic acid.

106. The synthetic peptide of any one of claims 95-100, wherein X₁ is an uncharged amino acid.

107. The synthetic peptide of claim 106, wherein X₁ is alanine, asparagine, cysteine, glycine, isoleucine, leucine, methionine, phenylalanine, proline, serine, threonine, tryptophan, tyrosine, or valine.

108. The synthetic peptide of any one of claims 95-100, wherein X₁ is an aliphatic amino acid.

109. The synthetic peptide of claim 108, wherein X₁ is selected from the group consisting of alanine, glycine, isoleucine, leucine, proline, and valine.

110. The synthetic peptide of claim any one of claims 95-100, wherein X₁ is isoleucine.

111. The synthetic peptide of claim 110, wherein the synthetic peptide comprises the amino acid sequence of SEQ ID NO: 5, 6, or 7:
(SEQ ID NO: 5)
PRPPKRI,
(SEQ ID NO: 6)
PRPPKRIL,
or
(SEQ ID NO: 7)
PRPPKRILE.

112. The synthetic peptide of any one of claims 95-100, wherein X₁ is an aromatic amino acid.

113. The synthetic peptide of claim 112, wherein X₁ is phenylalanine, tryptophan, or tyrosine.

114. The synthetic peptide of any one of claims 95-100, wherein X₁ is a sulfur-containing amino acid.

115. The synthetic peptide of claim 114, wherein X₁ is cysteine or methionine.

116. The synthetic peptide of any one of claims 95-100, wherein X₁ is an amino acid with a hydroxyl-containing side chain.

117. The synthetic peptide of claim 116, wherein X₁ is serine or threonine.

118. The synthetic peptide of any one of claims 95-100, wherein X₁ is an acidic amino acid.

119. The synthetic peptide of claim 118, wherein X₁ is aspartic acid or glutamic acid.

120. The synthetic peptide of any one of claims 95-100, wherein X₁ is a basic amino acid.

121. The synthetic peptide of claim 120, wherein X₁ is arginine, histidine, or lysine.

122. The synthetic peptide of any one of claims 95-100, wherein X₁ is an amino acid with an amide-containing sidechain.

123. The synthetic peptide of claim 122, wherein X₁ is asparagine or glutamine.

124. The synthetic peptide of any one of claims 95-100, wherein the synthetic peptide has the amino acid sequence of SEQ ID NO: 8, 9, or 10:
(SEQ ID NO: 8)
PRPPKR
(SEQ ID NO: 9)
PPRPPKR
(SEQ ID NO: 10)
PPPEHIPPPPRPPKR.

125. A synthetic peptide consisting essentially of an amino acid sequence of seven to ten consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid;
X₂ is selected from the group consisting of alanine, arginine, isoleucine, leucine, lysine, phenylalanine, proline, threonine, tyrosine, and valine;
if X₁ is alanine and X₂ is valine, then X₃ is not proline;
if X₂ is proline and X₃ is arginine, then X₁ is not arginine or glutamine; and
if X₂ is proline and X₃ is methionine, then X₁ is not histidine.

126. A synthetic peptide consisting essentially of an amino acid sequence of eleven consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is selected from the group consisting of alanine, arginine, isoleucine, leucine, lysine, phenylalanine, proline, threonine, tyrosine, and valine;
X₄ is either absent or any amino acid; and
if X₂ is proline and X₄ is not absent, then
(i) X₁ is not threonine;
(ii) X₃ is not lysine; and/or
(iii) X₄ is not serine.

127. A synthetic peptide consisting essentially of an amino acid sequence of twelve consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is selected from the group consisting of alanine, arginine, isoleucine, leucine, lysine, phenylalanine, proline, threonine, tyrosine, and valine;
X₄ is either absent or any amino acid;
if X₂ is proline and X₄ is not absent, then
(i) X₁ is not arginine;
(ii) X₃ is not threonine; and/or
(iii) X₄ is not threonine.

128. A synthetic peptide consisting essentially of an amino acid sequence of thirteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is selected from the group consisting of alanine, arginine, isoleucine, leucine, lysine, phenylalanine, proline, threonine, tyrosine, and valine; and
X₄ is either absent or any amino acid.

129. A synthetic peptide consisting essentially of an amino acid sequence of fourteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is selected from the group consisting of alanine, arginine, isoleucine, leucine, lysine, phenylalanine, proline, threonine, tyrosine, and valine;
X₄ is either absent or any amino acid;
if X₁ is alanine and X₂ is valine, then X₃ is not proline; and
if X₂ is proline and X₄ is absent, then:
(i) X₁ is not arginine; and/or
(ii) X₃ is not serine.

130. A synthetic peptide consisting essentially of an amino acid sequence of fifteen consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is selected from the group consisting of alanine, arginine, isoleucine, leucine, lysine, phenylalanine, proline, threonine, tyrosine, and valine;
X₄ is either absent or any amino acid; and
if X₂ is proline and X₄ is not absent, then:
(i) if X₁ is arginine, X₃ is lysine, and X₄ is isoleucine, then X₄ is not the C-terminal amino acid and/or the N-terminal amino acid is not a proline;
(ii) if X₄ is alanine, then X₁ is not proline and/or X₃ is not arginine; and
(iii) if X₄ is proline, then:
(a) if X₁ is methionine, then X₃ is not asparagine; and
(b) if X₁ is arginine, then X₃ is not glycine.

131. A synthetic peptide consisting essentially of an amino acid sequence of (i) sixteen to thirty (sixteen, seventeen, eighteen, nineteen, twenty, twenty-one, twenty-two, twenty-three, twenty-four, twenty-five, twenty-six, twenty-seven, twenty-eight, twenty-nine, or thirty) or (ii) sixteen to 50 (sixteen, seventeen, eighteen, nineteen, twenty, twenty-one, twenty-two, twenty-three, twenty-four, twenty-five, twenty-six, twenty-seven, twenty-eight, twenty-nine, thirty, 31, 32, 33, 34, 35, 36, 37, 38, 39, 40, 41, 42, 43, 44, 45, 46, 47, 48, 49, or 50) consecutive amino acids including an N-terminal amino acid and a C-terminal amino acid, wherein the amino acid sequence comprises the amino acid sequence of SEQ ID NO: 3:
(SEQ ID NO: 3)
PX₁X₂PX₃RX₄
wherein
X₁ and X₃ are each independently any amino acid;
X₂ is selected from the group consisting of alanine, arginine, isoleucine, leucine, lysine, phenylalanine, proline, threonine, tyrosine, and valine;
X₄ is either absent or any amino acid;
wherein, if the synthetic peptide consists essentially of twenty consecutive amino acids, then the four most C-terminal amino acid residues of the synthetic peptide is not SLSR (SEQ ID NO: 85).

132. The synthetic peptide of claim 131, consisting essentially of an amino acid sequence of sixteen to thirty consecutive amino acids.

133. The synthetic peptide of claim 131, consisting essentially of an amino acid sequence of sixteen to fifty consecutive amino acids.

134. The synthetic peptide of any one of claims 127-133, wherein X₂ is selected from the group consisting of alanine, arginine, isoleucine, leucine, lysine, proline, threonine, tyrosine, and valine.

135. The synthetic peptide of claim 134, wherein X₂ is selected from the group consisting of alanine, isoleucine, proline, and valine.

136. The synthetic peptide of claim 135, wherein X₂ is selected from the group consisting of isoleucine, proline, and valine.

137. The synthetic peptide of any one of claims 1-136, wherein the synthetic peptide exhibits binding to the SH3-2 domain of NCK1 with a K_{D} of ≤1,000 μM.

138. The synthetic peptide of claim 137, wherein the synthetic peptide exhibits binding to the SH3-2 domain of NCK1 with a K_{D} of <600 μM.

139. The synthetic peptide of claim 138, wherein the synthetic peptide exhibits binding to the SH3-2 domain of NCK1 with a K_{D} of <300 μM.

140. A synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to fifty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid;
X₂ is selected from the group consisting of selected from the group consisting of alanine, arginine, isoleucine, leucine, lysine, phenylalanine, proline, threonine, tyrosine, and valine;
and
the synthetic peptide exhibits binding to the SH3-2 domain of NCK1 with a K_{D} of <1,000 μM.

141. The synthetic peptide of claim 140, consisting essentially of an amino acid sequence of six to thirty consecutive amino acids.

142. The synthetic peptide of claim 140, consisting essentially of an amino acid sequence of six to fifty consecutive amino acids.

143. The synthetic peptide of any one of claims 140-142, wherein X₂ is selected from the group consisting of alanine, arginine, isoleucine, leucine, lysine, proline, threonine, tyrosine, and valine.

144. The synthetic peptide of claim 143, wherein X₂ is selected from the group consisting of alanine, isoleucine, proline, and valine.

145. The synthetic peptide of claim 144, wherein X₂ is selected from the group consisting of isoleucine, proline, and valine.

146. The synthetic peptide of any one of claims 142-145, consisting essentially of an amino acid sequence of at least seven, at least eight, at least nine, at least ten, at least eleven, at least twelve, at least thirteen, at least fourteen, or at least fifteen consecutive amino acids.

147. The synthetic peptide of any one of claims 142-145, wherein the synthetic peptide exhibits binding to the SH3-2 domain of NCK1 with a K_{D} of 600 μM.

148. The synthetic peptide of claim 147, wherein the synthetic peptide exhibits binding to the SH3-2 domain of NCK1 with a K_{D} of 300 μM.

149. The synthetic peptide of any one of claims 1-148, wherein at least one amino acid or two amino acid residues are covalently linked to a chemical moiety not present in a natural amino acid.

150. The synthetic peptide of claim 149, wherein the N-terminal amino acid comprises a modification.

151. The synthetic peptide of claim 150, wherein the N-terminal amino acid is acetylated.

152. The synthetic peptide of any one of claims 1-150, wherein the N-terminal amino acid is H-pyroglutamic acid (H-Pyr).

153. The synthetic peptide of any one of claims 1-152, wherein the C-terminal amino acid comprises a modification.

154. The synthetic peptide of claim 153, wherein the C-terminal amino acid is amidated.

155. The synthetic peptide of claim 153, wherein the C-terminal amino acid comprises an ethylamide (NHEt) group.

156. The synthetic peptide of any one of claims 1-155, comprising an amino acid residue (X₀) that is N-terminal to position 1 in SEQ ID NO: 1, 2, 3, or 4.

157. The synthetic peptide of claim 156, wherein X₀ selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

158. The synthetic peptide of claim 157, wherein X₀ is selected from the group consisting of alanine, arginine, asparagine, aspartic acid, cysteine, glutamic acid, glutamine, glycine, histidine, isoleucine, lysine, methionine, phenylalanine, proline, serine, threonine, tryptophan, tyrosine, and valine.

159. A composition comprising the synthetic peptide of any one of claims 1-158 and a salt.

160. The composition of claim 159, wherein the salt is an acetate.

161. The composition of claim 160, wherein the acetate is bis-acetate.

162. The composition of claim 160, wherein the acetate is tri-acetate.

163. A composition comprising a synthetic peptide of any one of claims 1-158, or a composition of any one of claims 159-162, wherein the purity of the synthetic peptide in the composition is at least 95% (e.g., 95%, 96%, 97%, 98%, 99%).

164. A pharmaceutical composition comprising the synthetic peptide of any one of claims 1-158, or the composition of any one of claims 159-163, and a pharmaceutically acceptable carrier.

165. A pharmaceutical composition comprising a synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to fifty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty, six to 31, six to 32, six to 33, six to 34, six to 35, six to 36, six to 37, six 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tyrosine, tryptophan, and valine;
X₂ is selected from the group consisting of alanine, arginine, isoleucine, leucine, lysine, phenylalanine, proline, threonine, tyrosine, and valine; and
X₃ is selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

166. The pharmaceutical composition of claim 165, wherein the pharmaceutical composition consists essentially of an amino acid sequence of six to thirty consecutive amino acids.

167. The pharmaceutical composition of claim 165, wherein the pharmaceutical composition consists essentially of an amino acid sequence of six to fifty consecutive amino acids.

168. The pharmaceutical composition of any one of claims 165-167, wherein X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, tryptophan, and valine.

169. The pharmaceutical composition of claim 168, wherein X₁ is selected from the group consisting of alanine, arginine, asparagine, aspartic acid, cysteine, glutamic acid, glutamine, histidine, isoleucine, leucine, lysine, methionine, phenylalanine, proline, serine, threonine, tyrosine, and valine.

170. The pharmaceutical composition of any one of claims 165-169, wherein X₂ is selected from the group consisting of alanine, arginine, isoleucine, leucine, lysine, proline, threonine, tyrosine, and valine.

171. The pharmaceutical composition of claim 170, wherein X₂ is selected from the group consisting of alanine, isoleucine, proline, and valine.

172. The pharmaceutical composition of claim 171, wherein X₂ is selected from the group consisting of isoleucine, proline, and valine.

173. The pharmaceutical composition of any one of claims 165-172, wherein X₃ is selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine.

174. The pharmaceutical composition of claim 173, wherein X₃ is selected from the group consisting of alanine, arginine, asparagine, cysteine, glutamic acid, glutamine, histidine, isoleucine, leucine, lysine, methionine, proline, serine, threonine, tryptophan, tyrosine, and valine.

175. The pharmaceutical composition of any one of claims 165-174, wherein the composition is sterile.

176. The pharmaceutical composition of any one of claims 165-175, wherein the composition is dried (e.g., freeze dried or lyophilized).

177. The pharmaceutical composition of any one of claims 165-175, wherein the composition is an aqueous composition.

178. The pharmaceutical composition of claim 177, wherein the aqueous composition is injectable.

179. The pharmaceutical composition of claim 177 or 178, wherein the concentration of the synthetic peptide is between about 150 mg/mL and about 400 mg/mL.

180. The pharmaceutical composition of claim 179, wherein the concentration of the synthetic peptide is between about 200 mg/mL and about 300 mg/mL.

181. A delivery system comprising (i) delivery device and (ii) a composition of any one of claims 159-163 or a pharmaceutical composition of any one of claims 164-180.

182. The delivery system of claim 181, wherein the delivery device comprises a syringe.

183. A method of modulating the activity of NCK1, the method comprising contacting NCK1 with a synthetic peptide of any one of claims 1-158 or the composition of any one of claims 159-163, thereby to modulate the activity of NCK1.

184. The method of claim 183, wherein the step of contacting disrupts the interaction between PTP1B and NCK1.

185. A method of modulating the activity of NCK1, the method comprising contacting NCK1 with an agent that disrupts an interaction between PTP1B and NCK1, thereby to modulate the activity of NCK1.

186. The method of claim 185, wherein the agent comprises a peptide.

187. The method of any one of claims 183-186, wherein the step of contacting results in modulating the activity of PTP1B.

188. A method of decreasing body fat mass in a subject in need thereof, the method comprising administering to the subject an effective amount of a synthetic peptide or a pharmaceutical composition comprising the synthetic peptide and a pharmaceutically acceptable carrier, thereby to decrease body fat mass of the subject,
the synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid; and
X₂ is a non-aromatic amino acid.

189. The method of claim 188, wherein the synthetic peptide consists essentially of an amino acid sequence of six to thirty consecutive amino acids.

190. The method of claim 188, wherein the synthetic peptide consists essentially of an amino acid sequence of six to fifty consecutive amino acids.

191. A method of decreasing body fat mass in a subject in need thereof, the method comprising administering to the subject an effective amount of a synthetic peptide or a pharmaceutical composition comprising the synthetic peptide and a pharmaceutically acceptable carrier, thereby to decrease body fat mass of the subject,
the synthetic peptide consisting essentially of an amino acid sequence of (i) six to 30 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid; and
X₂ is selected from the group consisting of alanine, arginine, isoleucine, leucine, lysine, phenylalanine, proline, threonine, tyrosine, and valine.

192. The method of claim 191, wherein the synthetic peptide consists essentially of an amino acid sequence of six to thirty consecutive amino acids.

193. The method of claim 191, wherein the synthetic peptide consists essentially of an amino acid sequence of six to fifty consecutive amino acids.

194. A method of decreasing body fat mass in a subject in need thereof, the method comprising administering to the subject an effective amount of a synthetic peptide of any one of claims 1-158, the composition of any one of claims 159-163, or the pharmaceutical composition of any one of claims 164-180, thereby to decrease body fat mass of the subject.

195. A method of treating or preventing obesity in a subject in need thereof, the method comprising administering to the subject an effective amount of a synthetic peptide or a pharmaceutical composition comprising the synthetic peptide and a pharmaceutically acceptable carrier, thereby to treat obesity in the subject,
the synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid; and
X₂ is a non-aromatic amino acid.

196. The method of claim 195, wherein the synthetic peptide consists essentially of an amino acid sequence of six to thirty consecutive amino acids.

197. The method of claim 195, wherein the synthetic peptide consists essentially of an amino acid sequence of six to fifty consecutive amino acids.

198. A method of treating or preventing obesity in a subject in need thereof, the method comprising administering to the subject an effective amount of a synthetic peptide or a pharmaceutical composition comprising the synthetic peptide and a pharmaceutically acceptable carrier, thereby to treat or prevent obesity in the subject,
the synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid; and
X₂ is selected from the group consisting of alanine, arginine, isoleucine, leucine, lysine, phenylalanine, proline, threonine, tyrosine, and valine.

199. The method of claim 198, wherein the synthetic peptide consists essentially of an amino acid sequence of six to thirty consecutive amino acids.

200. The method of claim 198, wherein the synthetic peptide consists essentially of an amino acid sequence of six to fifty consecutive amino acids.

201. A method of treating or preventing obesity in a subject in need thereof, the method comprising administering to the subject an effective amount of a synthetic peptide of any one of claims 1-144, the composition of any one of claims 145-149, or the pharmaceutical composition of any one of claims 150-165, thereby to treat obesity in the subject.

202. A method of treating or preventing diabetes in a subject in need thereof, the method comprising administering to the subject an effective amount of a synthetic peptide or a pharmaceutical composition comprising the synthetic peptide and a pharmaceutically acceptable carrier, thereby to treat or prevent diabetes in the subject,
the synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid; and
X₂ is a non-aromatic amino acid.

203. The method of claim 202, wherein the synthetic peptide consists essentially of an amino acid sequence of six to thirty consecutive amino acids.

204. The method of claim 202, wherein the synthetic peptide of consists essentially of an amino acid sequence of six to fifty consecutive amino acids.

205. A method of treating or preventing diabetes in a subject in need thereof, the method comprising administering to the subject an effective amount of a synthetic peptide or a pharmaceutical composition comprising the synthetic peptide and a pharmaceutically acceptable carrier, thereby to treat or prevent diabetes in the subject,
the synthetic peptide consisting essentially of an amino acid sequence of (i) six to thirty (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, or six to thirty) or (ii) six to 50 (six to fifteen, six to sixteen, six to seventeen, six to eighteen, six to nineteen, six to twenty, six to twenty-one, six to twenty-two, six to twenty-three, six to twenty-four, six to twenty-five, six to twenty-six, six to twenty-seven, six to twenty-eight, six to twenty-nine, six to thirty, six to 31, six to 32, six to 33, six to 34, six to 35, six to 36, six to 37, six to 38, six to 39, six to 40, six to 41, six to 42, six to 43, six to 44, six to 45, six to 46, six to 47, six to 48, six to 49, or six to 50) consecutive amino acids including an N-terminal amino acid and a C-terminal amino acid, wherein the amino acid sequence comprises the amino acid sequence of SEQ ID NO: 2:
(SEQ ID NO: 2)
PX₁X₂PX₃R
wherein
X₁ and X₃ are each independently any amino acid; and
X₂ is selected from the group consisting of alanine, arginine, isoleucine, leucine, lysine, phenylalanine, proline, threonine, tyrosine, and valine.

206. The method of claim 205, wherein the synthetic peptide consists essentially of an amino acid sequence of six to thirty consecutive amino acids.

207. The method of claim 205, wherein the synthetic peptide consists essentially of an amino acid sequence of six to fifty consecutive amino acids.

208. A method of treating or preventing diabetes in a subject in need thereof, the method comprising administering to the subject an effective amount of a synthetic peptide of any one of claims 1-158, the composition of any one of claims 159-163, or a pharmaceutical composition of any one of claims 164-180, thereby to treat or prevent diabetes in the subject.

209. The method of any one of claims 205-208, wherein the diabetes is type 2 diabetes.

210. The method of any one of claims 191-193, 198-200, or 205-207, wherein X₂ is selected from the group consisting of alanine, arginine, isoleucine, leucine, lysine, proline, threonine, tyrosine, and valine.

211. The method of claim 210, wherein X₂ is selected from the group consisting of alanine, isoleucine, proline, and valine.

212. The method of claim 211, wherein X₂ is selected from the group consisting of isoleucine, proline, and valine.

213. The method of any one of claims 191-193, 198-200, 205-207, or 210-212, consisting essentially of an amino acid sequence of at least seven, at least eight, at least nine, at least ten, at least eleven, at least twelve, at least thirteen, at least fourteen, or at least fifteen consecutive amino acids.

214. The method of any one of claims 188-213, wherein the subject is a mammal.

215. The method of claim 214, wherein the mammal is a human.

216. The method of any one of claims 188-215, wherein the administration comprises systemic administration.

217. The method of claim 216, wherein the administration comprises subcutaneous administration.

218. The method of any one of claims 188-217, wherein X₁ is a charged amino acid.

219. The method of claim 218, wherein X₁ is a positively charged amino acid.

220. The method of claim 219, wherein X₁ is arginine, histidine, or lysine.

221. The method of claim 218, wherein X₁ is a negatively charged amino acid.

222. The method of claim 221, wherein X₁ is glutamic acid.

223. The method of any one of claims 188-217, wherein X₁ is an uncharged amino acid.

224. The method of claim 223, wherein X₁ is a hydrophobic amino acid.

225. The method of claim 223, wherein X₁ is selected from the group consisting of alanine, asparagine, and serine.

226. The method of any one of claims 188-190, 194, 195-197, 201, 202-204, 208, or 209, wherein X₂ is an uncharged, non-aromatic amino acid.

227. The method of claim 226, wherein X₂ is alanine or proline.

228. The method of claim 226, wherein X₂ is a hydrophobic, non-aromatic amino acid.

229. The method of claim 228, wherein X₂ is alanine.

230. The method of any one of claims 188-190, 194, 195-197, 201, 202-204, 208, or 209, wherein X₂ is a charged amino acid.

231. The method of claim 230, wherein X₂ is a positively charged amino acid.

232. The method of claim 231, wherein X₂ is arginine.

233. The method of any one of claims 188-232, wherein X₃ is a charged amino acid.

234. The method of claim 233, wherein X₃ is a positively charged amino acid.

235. The method of claim 234, wherein X₃ is arginine, histidine, or lysine.

236. The method of claim 235, wherein X₃ is a negatively charged amino acid.

237. The method of claim 236, wherein X₃ is glutamic acid.

238. The method of any one of claims 188-232, wherein X₃ is an uncharged amino acid.

239. The method of claim 238, wherein X₃ is selected from the group consisting of alanine, asparagine, cysteine, serine, and tryptophan.

240. The method of claim 238, wherein X₃ is a hydrophobic amino acid.

241. The method of claim 240, wherein X₃ is selected from the group consisting of alanine, cysteine, and tryptophan.


SEQUENCE LISTING (170 sequences)

SEQ ID NO: 1 - protein, 6 aa, synthetic construct
  SITE 1: note = Optional N-terminal acetylation
  VARIANT 2: note = Any amino acid
  VARIANT 5: note = Any amino acid
  SITE 6: note = Optional C-terminal amidation
PXPPXR

SEQ ID NO: 2-3 - skipped (2 entries)

SEQ ID NO: 4 - protein, 7 aa, synthetic construct
  VARIANT 7: note = Any amino acid or absent
PRPPKRX

SEQ ID NO: 5 - protein, 7 aa, synthetic construct
PRPPKRI

SEQ ID NO: 6 - protein, 8 aa, synthetic construct
PRPPKRIL

SEQ ID NO: 7 - protein, 9 aa, synthetic construct
PRPPKRILE

SEQ ID NO: 8 - protein, 6 aa, synthetic construct
  SITE 1: note = Optional N-terminal acetylation
  SITE 6: note = Optional C-terminal amidation or ethyl amidation
PRPPKR

SEQ ID NO: 9 - protein, 7 aa, synthetic construct
  SITE 1: note = Optional N-terminal acetylation
  SITE 7: note = Optional C-terminal amidation or ethyl amidation
PPRPPKR

SEQ ID NO: 10 - protein, 15 aa, synthetic construct
  SITE 1: note = Optional N-terminal acetylation
  SITE 15: note = Optional C-terminal amidation
PPPEHIPPPPRPPKR

SEQ ID NO: 11 - protein, 8 aa, synthetic construct
  SITE 1: note = Optional N-terminal acetylation
  SITE 8: note = Optional C-terminal amidation
PPPRPPKR

SEQ ID NO: 12 - protein, 9 aa, synthetic construct
  SITE 1: note = Optional N-terminal acetylation
  SITE 9: note = Optional C-terminal amidation
PPPPRPPKR

SEQ ID NO: 13 - protein, 10 aa, synthetic construct
  SITE 1: note = Optional N-terminal acetylation
  SITE 10: note = Optional C-terminal amidation
IPPPPRPPKR

SEQ ID NO: 14 - protein, 11 aa, synthetic construct
  SITE 1: note = Optional N-terminal acetylation
  SITE 11: note = Optional C-terminal amidation
HIPPPPRPPKR

SEQ ID NO: 15 - protein, 12 aa, synthetic construct
  SITE 1: note = Optional N-terminal acetylation
  SITE 12: note = Optional C-terminal amidation
EHIPPPPRPPKR

SEQ ID NO: 16 - protein, 13 aa, synthetic construct
  SITE 1: note = Optional N-terminal acetylation
  SITE 13: note = Optional C-terminal amidation
PEHIPPPPRPPKR

SEQ ID NO: 17 - protein, 14 aa, synthetic construct
  SITE 1: note = Optional N-terminal acetylation
  SITE 14: note = Optional C-terminal amidation
PPEHIPPPPRPPKR

SEQ ID NO: 18 - protein, 8 aa, synthetic construct
  SITE 1: note = Optional N-terminal acetylation
  SITE 8: note = Optional C-terminal amidation
PPRPPKRI

SEQ ID NO: 19 - protein, 9 aa, synthetic construct
  SITE 1: note = Optional N-terminal acetylation
  SITE 9: note = Optional C-terminal amidation
PPRPPKRIL

SEQ ID NO: 20 - protein, 10 aa, synthetic construct
  SITE 1: note = Optional N-terminal acetylation
  SITE 10: note = Optional C-terminal amidation
PPRPPKRILE

SEQ ID NO: 21 - protein, 18 aa, synthetic construct
  SITE 1: note = Optional N-terminal acetylation
  SITE 18: note = Optional C-terminal amidation
PPPEHIPPPPRPPKRILE

SEQ ID NO: 22 - protein, 15 aa, synthetic construct
  SITE 1: note = Optional N-terminal acetylation
  SITE 15: note = Optional C-terminal amidation
APPEHIPPPPRPPKR

SEQ ID NO: 23 - protein, 15 aa, synthetic construct
  SITE 1: note = Optional N-terminal acetylation
  SITE 15: note = Optional C-terminal amidation
PAPEHIPPPPRPPKR

SEQ ID NO: 24 - protein, 15 aa, synthetic construct
  SITE 1: note = Optional N-terminal acetylation
  SITE 15: note = Optional C-terminal amidation
PPAEHIPPPPRPPKR

SEQ ID NO: 25 - protein, 15 aa, synthetic construct
  SITE 1: note = Optional N-terminal acetylation
  SITE 15: note = Optional C-terminal amidation
PPPAHIPPPPRPPKR

SEQ ID NO: 26 - protein, 15 aa, synthetic construct
  SITE 1: note = Optional N-terminal acetylation
  SITE 15: note = Optional C-terminal amidation
PPPEAIPPPPRPPKR

SEQ ID NO: 27 - protein, 15 aa, synthetic construct
  SITE 1: note = Optional N-terminal acetylation
  SITE 15: note = Optional C-terminal amidation
PPPEHAPPPPRPPKR

SEQ ID NO: 28 - protein, 15 aa, synthetic construct
  SITE 1: note = Optional N-terminal acetylation
  SITE 15: note = Optional C-terminal amidation
PPPEHIAPPPRPPKR

SEQ ID NO: 29 - protein, 15 aa, synthetic construct
  SITE 1: note = Optional N-terminal acetylation
  SITE 15: note = Optional C-terminal amidation
PPPEHIPAPPRPPKR

SEQ ID NO: 30 - protein, 15 aa, synthetic construct
  SITE 1: note = Optional N-terminal acetylation
  SITE 15: note = Optional C-terminal amidation
PPPEHIPPAPRPPKR

SEQ ID NO: 31 - protein, 15 aa, synthetic construct
  SITE 1: note = Optional N-terminal acetylation
  SITE 15: note = Optional C-terminal amidation
PPPEHIPPPARPPKR

SEQ ID NO: 32 - protein, 15 aa, synthetic construct
  SITE 1: note = Optional N-terminal acetylation
  SITE 15: note = Optional C-terminal amidation
PPPEHIPPPPAPPKR

SEQ ID NO: 33 - protein, 15 aa, synthetic construct
  SITE 1: note = Optional N-terminal acetylation
  SITE 15: note = Optional C-terminal amidation
PPPEHIPPPPRAPKR

SEQ ID NO: 34 - protein, 15 aa, synthetic construct
  SITE 1: note = Optional N-terminal acetylation
  SITE 15: note = Optional C-terminal amidation
PPPEHIPPPPRPAKR

SEQ ID NO: 35 - protein, 15 aa, synthetic construct
  SITE 1: note = Optional N-terminal acetylation
  SITE 15: note = Optional C-terminal amidation
PPPEHIPPPPRPPAR

SEQ ID NO: 36 - protein, 15 aa, synthetic construct
  SITE 1: note = Optional N-terminal acetylation
  SITE 15: note = Optional C-terminal amidation
PPPEHIPPPPRPPKA

SEQ ID NO: 37 - protein, 6 aa, synthetic construct
  SITE 1: note = Optional N-terminal acetylation
  SITE 6: note = Optional C-terminal amidation
ARPPKR

SEQ ID NO: 38 - protein, 6 aa, synthetic construct
  SITE 1: note = Optional N-terminal acetylation
  SITE 6: note = Optional C-terminal amidation
PAPPKR

SEQ ID NO: 39 - protein, 6 aa, synthetic construct
  SITE 1: note = Optional N-terminal acetylation
  SITE 6: note = Optional C-terminal amidation
PRAPKR

SEQ ID NO: 40 - protein, 6 aa, synthetic construct
  SITE 1: note = Optional N-terminal acetylation
  SITE 6: note = Optional C-terminal amidation
PRPAKR

SEQ ID NO: 41 - protein, 6 aa, synthetic construct
  SITE 1: note = Optional N-terminal acetylation
  SITE 6: note = Optional C-terminal amidation
PRPPAR

SEQ ID NO: 42 - protein, 6 aa, synthetic construct
  SITE 1: note = Optional N-terminal acetylation
  SITE 6: note = Optional C-terminal amidation
PRPPKA

SEQ ID NO: 43-44 - skipped (2 entries)

SEQ ID NO: 45 - protein, 4 aa, synthetic construct
  SITE 1: note = Optional N-terminal acetylation
  SITE 4: note = Optional C-terminal amidation
RPPK

SEQ ID NO: 46 - protein, 4 aa, synthetic construct
  SITE 1: note = Optional N-terminal acetylation
  SITE 4: note = Optional C-terminal amidation
PPKR

SEQ ID NO: 47 - protein, 5 aa, synthetic construct
  SITE 1: note = Optional N-terminal acetylation
  SITE 5: note = Optional C-terminal amidation
RPPKR

SEQ ID NO: 48-49 - skipped (2 entries)

SEQ ID NO: 50 - protein, 4 aa, synthetic construct
  SITE 1: note = Optional N-terminal acetylation
  SITE 4: note = Optional C-terminal amidation
PRPP

SEQ ID NO: 51 - protein, 5 aa, synthetic construct
  SITE 1: note = Optional N-terminal acetylation
  SITE 5: note = Optional C-terminal amidation
PRPPK

SEQ ID NO: 52 - protein, 6 aa, synthetic construct
  SITE 1: note = Optional N-terminal acetylation
  SITE 6: note = Optional C-terminal amidation
PPRPPK

SEQ ID NO: 53 - protein, 6 aa, synthetic construct
  SITE 1: note = Optional N-terminal acetylation
  SITE 6: note = Optional C-terminal amidation
PPPRPP

SEQ ID NO: 54 - protein, 6 aa, synthetic construct
  SITE 1: note = Optional N-terminal acetylation
  SITE 6: note = Optional C-terminal amidation
PPPPRP

SEQ ID NO: 55 - protein, 6 aa, synthetic construct
  SITE 1: note = Optional N-terminal acetylation
  SITE 6: note = Optional C-terminal amidation
IPPPPR

SEQ ID NO: 56 - protein, 6 aa, synthetic construct
  SITE 1: note = Optional N-terminal acetylation
  SITE 6: note = Optional C-terminal amidation
EHIPPP

SEQ ID NO: 57 - protein, 6 aa, synthetic construct
  SITE 1: note = Optional N-terminal acetylation
  SITE 6: note = Optional C-terminal amidation
PPEHIP

SEQ ID NO: 58 - protein, 7 aa, synthetic construct
  SITE 1: note = Optional N-terminal acetylation
  SITE 7: note = Optional C-terminal amidation
PPPRPPK

SEQ ID NO: 59 - protein, 7 aa, synthetic construct
  SITE 1: note = Optional N-terminal acetylation
  SITE 7: note = Optional C-terminal amidation
APRPPKR

SEQ ID NO: 60 - protein, 7 aa, synthetic construct
  SITE 1: note = Optional N-terminal acetylation
  SITE 7: note = Optional C-terminal amidation
PPRPPKK

SEQ ID NO: 61 - protein, 7 aa, synthetic construct
  SITE 1: note = Optional N-terminal acetylation
  SITE 7: note = Optional C-terminal amidation
RPRPPKR

SEQ ID NO: 62 - protein, 7 aa, synthetic construct
  SITE 1: note = Optional N-terminal acetylation
  SITE 7: note = Optional C-terminal amidation
NPRPPKR

SEQ ID NO: 63 - protein, 7 aa, synthetic construct
  SITE 1: note = Optional N-terminal acetylation
  SITE 7: note = Optional C-terminal amidation
CPRPPKR

SEQ ID NO: 64 - protein, 7 aa, synthetic construct
  SITE 1: note = Optional N-terminal acetylation
  SITE 7: note = Optional C-terminal amidation
EPRPPKR

SEQ ID NO: 65 - protein, 7 aa, synthetic construct
  SITE 1: note = Optional N-terminal acetylation
  SITE 7: note = Optional C-terminal amidation
HPRPPKR

SEQ ID NO: 66 - protein, 7 aa, synthetic construct
  SITE 1: note = Optional N-terminal acetylation
  SITE 7: note = Optional C-terminal amidation
FPRPPKR

SEQ ID NO: 67 - protein, 7 aa, synthetic construct
  SITE 1: note = Optional N-terminal acetylation
  SITE 7: note = Optional C-terminal amidation
SPRPPKR

SEQ ID NO: 68 - protein, 7 aa, synthetic construct
  SITE 1: note = Optional N-terminal acetylation
  SITE 7: note = Optional C-terminal amidation
PPKPPKR

SEQ ID NO: 69 - protein, 7 aa, synthetic construct
  SITE 1: note = Optional N-terminal acetylation
  SITE 7: note = Optional C-terminal amidation
PPFPPKR

SEQ ID NO: 70 - protein, 7 aa, synthetic construct
  SITE 1: note = Optional N-terminal acetylation
  SITE 7: note = Optional C-terminal amidation
PPHPPKR

SEQ ID NO: 71 - protein, 7 aa, synthetic construct
  SITE 1: note = Optional N-terminal acetylation
  SITE 7: note = Optional C-terminal amidation
PPEPPKR

SEQ ID NO: 72 - protein, 7 aa, synthetic construct
  SITE 1: note = Optional N-terminal acetylation
  SITE 7: note = Optional C-terminal amidation
PPSPPKR

SEQ ID NO: 73 - protein, 7 aa, synthetic construct
  SITE 1: note = Optional N-terminal acetylation
  SITE 7: note = Optional C-terminal amidation
PPNPPKR

SEQ ID NO: 74 - protein, 7 aa, synthetic construct
  SITE 1: note = Optional N-terminal acetylation
  SITE 7: note = Optional C-terminal amidation
PPCPPKR

SEQ ID NO: 75 - protein, 7 aa, synthetic construct
  SITE 1: note = Optional N-terminal acetylation
  SITE 7: note = Optional C-terminal amidation
PPRRPKR

SEQ ID NO: 76 - protein, 7 aa, synthetic construct
  SITE 1: note = Optional N-terminal acetylation
  SITE 7: note = Optional C-terminal amidation
PPRFPKR

SEQ ID NO: 77 - protein, 7 aa, synthetic construct
  SITE 1: note = Optional N-terminal acetylation
  SITE 7: note = Optional C-terminal amidation
PPRPPRR

SEQ ID NO: 78 - protein, 7 aa, synthetic construct
  SITE 1: note = Optional N-terminal acetylation
  SITE 7: note = Optional C-terminal amidation
PPRPPHR

SEQ ID NO: 79 - protein, 7 aa, synthetic construct
  SITE 1: note = Optional N-terminal acetylation
  SITE 7: note = Optional C-terminal amidation
PPRPPWR

SEQ ID NO: 80 - protein, 7 aa, synthetic construct
  SITE 1: note = Optional N-terminal acetylation
  SITE 7: note = Optional C-terminal amidation
PPRPPER

SEQ ID NO: 81 - protein, 7 aa, synthetic construct
  SITE 1: note = Optional N-terminal acetylation
  SITE 7: note = Optional C-terminal amidation
PPRPPSR

SEQ ID NO: 82 - protein, 7 aa, synthetic construct
  SITE 1: note = Optional N-terminal acetylation
  SITE 7: note = Optional C-terminal amidation
PPRPPNR

SEQ ID NO: 83 - protein, 7 aa, synthetic construct
  SITE 1: note = Optional N-terminal acetylation
  SITE 7: note = Optional C-terminal amidation
PPRPPCR

SEQ ID NO: 84 - protein, 8 aa, synthetic construct
PPEHIPPP

SEQ ID NO: 85 - protein, 4 aa, synthetic construct
SLSR

SEQ ID NO: 86 - protein, 7 aa, synthetic construct
  SITE 1: note = Optional N-terminal acetylation
  SITE 7: note = Optional C-terminal amidation
PPRGPKR

SEQ ID NO: 87 - protein, 7 aa, synthetic construct
  SITE 1: note = Optional N-terminal acetylation
  SITE 7: note = Optional C-terminal amidation
PPRIPKR

SEQ ID NO: 88 - protein, 7 aa, synthetic construct
  SITE 1: note = Optional N-terminal acetylation
  SITE 7: note = Optional C-terminal amidation
PPRLPKR

SEQ ID NO: 89 - protein, 7 aa, synthetic construct
  SITE 1: note = Optional N-terminal acetylation
  SITE 7: note = Optional C-terminal amidation
PPRKPKR

SEQ ID NO: 90 - protein, 7 aa, synthetic construct
  SITE 1: note = Optional N-terminal acetylation
  SITE 7: note = Optional C-terminal amidation
PPRSPKR

SEQ ID NO: 91 - protein, 7 aa, synthetic construct
  SITE 1: note = Optional N-terminal acetylation
  SITE 7: note = Optional C-terminal amidation
PPRNPKR

SEQ ID NO: 92 - protein, 7 aa, synthetic construct
  SITE 1: note = Optional N-terminal acetylation
  SITE 7: note = Optional C-terminal amidation
PPRDPKR

SEQ ID NO: 93 - protein, 7 aa, synthetic construct
  SITE 1: note = Optional N-terminal acetylation
  SITE 7: note = Optional C-terminal amidation
PPRCPKR

SEQ ID NO: 94 - protein, 7 aa, synthetic construct
  SITE 1: note = Optional N-terminal acetylation
  SITE 7: note = Optional C-terminal amidation
PPREPKR

SEQ ID NO: 95 - protein, 7 aa, synthetic construct
  SITE 1: note = Optional N-terminal acetylation
  SITE 7: note = Optional C-terminal amidation
PPRQPKR

SEQ ID NO: 96 - protein, 7 aa, synthetic construct
  SITE 1: note = Optional N-terminal acetylation
  SITE 7: note = Optional C-terminal amidation
PPRHPKR

SEQ ID NO: 97 - protein, 7 aa, synthetic construct
  SITE 1: note = Optional N-terminal acetylation
  SITE 7: note = Optional C-terminal amidation
PPRMPKR

SEQ ID NO: 98 - protein, 7 aa, synthetic construct
  SITE 1: note = Optional N-terminal acetylation
  SITE 7: note = Optional C-terminal amidation
PPRTPKR

SEQ ID NO: 99 - protein, 7 aa, synthetic construct
  SITE 1: note = Optional N-terminal acetylation
  SITE 7: note = Optional C-terminal amidation
PPRWPKR

SEQ ID NO: 100 - protein, 7 aa, synthetic construct
  SITE 1: note = Optional N-terminal acetylation
  SITE 7: note = Optional C-terminal amidation
PPRYPKR

SEQ ID NO: 101 - protein, 7 aa, synthetic construct
  SITE 1: note = Optional N-terminal acetylation
  SITE 7: note = Optional C-terminal amidation
PPRVPKR

SEQ ID NO: 102 - protein, 7 aa, synthetic construct
  SITE 1: note = Optional N-terminal acetylation
  SITE 7: note = Optional C-terminal amidation
PPRPPGR

SEQ ID NO: 103 - protein, 7 aa, synthetic construct
  SITE 1: note = Optional N-terminal acetylation
  SITE 7: note = Optional C-terminal amidation
PPRPPIR

SEQ ID NO: 104 - protein, 7 aa, synthetic construct
  SITE 1: note = Optional N-terminal acetylation
  SITE 7: note = Optional C-terminal amidation
PPRPPLR

SEQ ID NO: 105 - protein, 7 aa, synthetic construct
  SITE 1: note = Optional N-terminal acetylation
  SITE 7: note = Optional C-terminal amidation
PPRPPFR

SEQ ID NO: 106 - protein, 7 aa, synthetic construct
  SITE 1: note = Optional N-terminal acetylation
  SITE 7: note = Optional C-terminal amidation
PPRPPDR

SEQ ID NO: 107 - protein, 7 aa, synthetic construct
  SITE 1: note = Optional N-terminal acetylation
  SITE 7: note = Optional C-terminal amidation
PPRPPMR

SEQ ID NO: 108 - protein, 7 aa, synthetic construct
  SITE 1: note = Optional N-terminal acetylation
  SITE 7: note = Optional C-terminal amidation
PPRPPQR

SEQ ID NO: 109 - protein, 7 aa, synthetic construct
  SITE 1: note = Optional N-terminal acetylation
  SITE 7: note = Optional C-terminal amidation
PPRPPPR

SEQ ID NO: 110 - protein, 7 aa, synthetic construct
  SITE 1: note = Optional N-terminal acetylation
  SITE 7: note = Optional C-terminal amidation
PPRPPTR

SEQ ID NO: 111 - protein, 7 aa, synthetic construct
  SITE 1: note = Optional N-terminal acetylation
  SITE 7: note = Optional C-terminal amidation
PPRPPYR

SEQ ID NO: 112 - protein, 7 aa, synthetic construct
  SITE 1: note = Optional N-terminal acetylation
  SITE 7: note = Optional C-terminal amidation
PPRPPVR

SEQ ID NO: 113 - protein, 7 aa, synthetic construct
  SITE 1: note = Optional N-terminal acetylation
  SITE 7: note = Optional C-terminal amidation
PPDPPKR

SEQ ID NO: 114 - protein, 7 aa, synthetic construct
  SITE 1: note = Optional N-terminal acetylation
  SITE 7: note = Optional C-terminal amidation
PPQPPKR

SEQ ID NO: 115 - protein, 7 aa, synthetic construct
  SITE 1: note = Optional N-terminal acetylation
  SITE 7: note = Optional C-terminal amidation
PPGPPKR

SEQ ID NO: 116 - protein, 7 aa, synthetic construct
  SITE 1: note = Optional N-terminal acetylation
  SITE 7: note = Optional C-terminal amidation
PPLPPKR

SEQ ID NO: 117 - protein, 7 aa, synthetic construct
  SITE 1: note = Optional N-terminal acetylation
  SITE 7: note = Optional C-terminal amidation
PPIPPKR

SEQ ID NO: 118 - protein, 7 aa, synthetic construct
  SITE 1: note = Optional N-terminal acetylation
  SITE 7: note = Optional C-terminal amidation
PPPPPKR

SEQ ID NO: 119 - protein, 7 aa, synthetic construct
  SITE 1: note = Optional N-terminal acetylation
  SITE 7: note = Optional C-terminal amidation
PPMPPKR

SEQ ID NO: 120 - protein, 7 aa, synthetic construct
  SITE 1: note = Optional N-terminal acetylation
  SITE 7: note = Optional C-terminal amidation
PPTPPKR

SEQ ID NO: 121 - protein, 7 aa, synthetic construct
  SITE 1: note = Optional N-terminal acetylation
  SITE 7: note = Optional C-terminal amidation
PPWPPKR

SEQ ID NO: 122 - protein, 7 aa, synthetic construct
  SITE 1: note = Optional N-terminal acetylation
  SITE 7: note = Optional C-terminal amidation
PPYPPKR

SEQ ID NO: 123 - protein, 7 aa, synthetic construct
  SITE 1: note = Optional N-terminal acetylation
  SITE 7: note = Optional C-terminal amidation
PPVPPKR

SEQ ID NO: 124 - protein, 7 aa, synthetic construct
  SITE 1: note = Optional N-terminal acetylation
  SITE 7: note = Optional C-terminal amidation
DPRPPKR

SEQ ID NO: 125 - protein, 7 aa, synthetic construct
  SITE 1: note = Optional N-terminal acetylation
  SITE 7: note = Optional C-terminal amidation
QPRPPKR

SEQ ID NO: 126 - protein, 7 aa, synthetic construct
  SITE 1: note = Optional N-terminal acetylation
  SITE 7: note = Optional C-terminal amidation
GPRPPKR

SEQ ID NO: 127 - protein, 7 aa, synthetic construct
  SITE 1: note = Optional N-terminal acetylation
  SITE 7: note = Optional C-terminal amidation
IPRPPKR

SEQ ID NO: 128 - protein, 7 aa, synthetic construct
  SITE 1: note = Optional N-terminal acetylation
  SITE 7: note = Optional C-terminal amidation
LPRPPKR

SEQ ID NO: 129 - protein, 7 aa, synthetic construct
  SITE 1: note = Optional N-terminal acetylation
  SITE 7: note = Optional C-terminal amidation
KPRPPKR

SEQ ID NO: 130 - protein, 7 aa, synthetic construct
  SITE 1: note = Optional N-terminal acetylation
  SITE 7: note = Optional C-terminal amidation
MPRPPKR

SEQ ID NO: 131 - protein, 7 aa, synthetic construct
  SITE 1: note = Optional N-terminal acetylation
  SITE 7: note = Optional C-terminal amidation
TPRPPKR

SEQ ID NO: 132 - protein, 7 aa, synthetic construct
  SITE 1: note = Optional N-terminal acetylation
  SITE 7: note = Optional C-terminal amidation
WPRPPKR

SEQ ID NO: 133 - protein, 7 aa, synthetic construct
  SITE 1: note = Optional N-terminal acetylation
  SITE 7: note = Optional C-terminal amidation
YPRPPKR

SEQ ID NO: 134 - protein, 7 aa, synthetic construct
  SITE 1: note = Optional N-terminal acetylation
  SITE 7: note = Optional C-terminal amidation
VPRPPKR

SEQ ID NO: 135 - protein, 20 aa, synthetic construct
  SITE 1: note = Optional N-terminal acetylation
  SITE 20: note = Optional C-terminal amidation
PPPEHIPPPPRPPKRILEPH

SEQ ID NO: 136 - protein, 20 aa, synthetic construct
  SITE 1: note = Optional N-terminal acetylation
  SITE 20: note = Optional C-terminal amidation
HEDLEPPPEHIPPPPRPPKR

SEQ ID NO: 137 - protein, 25 aa, synthetic construct
  SITE 1: note = Optional N-terminal acetylation
  SITE 25: note = Optional C-terminal amidation
PPPEHIPPPPRPPKRILEPHNGKCR

SEQ ID NO: 138 - protein, 7 aa, synthetic construct
  SITE 1: note = Optional N-terminal acetylation
  SITE 7: note = Optional C-terminal amidation
PAVPPRP

SEQ ID NO: 139 - protein, 12 aa, synthetic construct
  SITE 1: note = Optional N-terminal acetylation
  SITE 12: note = Optional C-terminal amidation
SPAKGEPSLPEK

SEQ ID NO: 140 - protein, 377 aa, Homo sapiens
MAEEVVVVAKFDYVAQQEQELDIKKNERLWLLDDSKSWWRVRNSMNKTGFVPSNYVERKNSARKASIVKNLKDTLGIGKVKRKPSVPDSASPADDSFVDPGERLYDLNMPAYVKFNYMAEREDELSLIKGTKVIVMEKCSDGWWRGSYNGQVGWFPSNYVTEEGDSPLGDHVGSLSEKLAAVVNNLNTGQVLHVVQALYPFSSSNDEELNFEKGDVMDVIEKPENDPEWWKCRKINGMVGLVPKNYVTVMQNNPLTSGLEPSPPQCDYIRPSLTGKFAGNPWYYGKVTRHQAEMALNERGHEGDFLIRDSESSPNDFSVSLKAQGKNKHFKVQLKETVYCIGQRKFSTMEELVEHYKKAPIFTSEQGEKLYLVKHLS

SEQ ID NO: 141 - protein, 30 aa, synthetic construct
  SITE 1: note = Optional N-terminal acetylation
  SITE 30: note = Optional C-terminal amidation
PPPEHIPPPPRPPKRILEPHNGKCREFFPN

SEQ ID NO: 142 - protein, 25 aa, synthetic construct
  SITE 1: note = Optional N-terminal acetylation
  SITE 25: note = Optional C-terminal amidation
WKELSHEDLEPPPEHIPPPPRPPKR

SEQ ID NO: 143 - protein, 30 aa, synthetic construct
  SITE 1: note = Optional N-terminal acetylation
  SITE 30: note = Optional C-terminal amidation
SVQDQWKELSHEDLEPPPEHIPPPPRPPKR

SEQ ID NO: 144 - protein, 8 aa, synthetic construct
  SITE 1: note = Optional N-terminal acetylation
  SITE 8: note = Optional C-terminal amidation
PPRPPKRA

SEQ ID NO: 145 - protein, 8 aa, synthetic construct
  SITE 1: note = Optional N-terminal acetylation
  SITE 8: note = Optional C-terminal amidation
PPRPPKRR

SEQ ID NO: 146 - protein, 8 aa, synthetic construct
  SITE 1: note = Optional N-terminal acetylation
  SITE 8: note = Optional C-terminal amidation
PPRPPKRN

SEQ ID NO: 147 - protein, 8 aa, synthetic construct
  SITE 1: note = Optional N-terminal acetylation
  SITE 8: note = Optional C-terminal amidation
PPRPPKRD

SEQ ID NO: 148 - protein, 8 aa, synthetic construct
  SITE 1: note = Optional N-terminal acetylation
  SITE 8: note = Optional C-terminal amidation
PPRPPKRC

SEQ ID NO: 149 - protein, 8 aa, synthetic construct
  SITE 1: note = Optional N-terminal acetylation
  SITE 8: note = Optional C-terminal amidation
PPRPPKRF

SEQ ID NO: 150 - protein, 8 aa, synthetic construct
  SITE 1: note = Optional N-terminal acetylation
  SITE 8: note = Optional C-terminal amidation
PPRPPKRS

SEQ ID NO: 151 - protein, 8 aa, synthetic construct
  SITE 1: note = Optional N-terminal acetylation
  SITE 8: note = Optional C-terminal amidation
PPRPPKRG

SEQ ID NO: 152 - protein, 8 aa, synthetic construct
  SITE 1: note = Optional N-terminal acetylation
  SITE 8: note = Optional C-terminal amidation
PPRPPKRH

SEQ ID NO: 153 - protein, 8 aa, synthetic construct
  SITE 1: note = Optional N-terminal acetylation
  SITE 8: note = Optional C-terminal amidation
PPRPPKRL

SEQ ID NO: 154 - protein, 8 aa, synthetic construct
  SITE 1: note = Optional N-terminal acetylation
  SITE 8: note = Optional C-terminal amidation
PPRPPKRK

SEQ ID NO: 155 - protein, 8 aa, synthetic construct
  SITE 1: note = Optional N-terminal acetylation
  SITE 8: note = Optional C-terminal amidation
PPRPPKRM

SEQ ID NO: 156 - protein, 8 aa, synthetic construct
  SITE 1: note = Optional N-terminal acetylation
  SITE 8: note = Optional C-terminal amidation
PPRPPKRP

SEQ ID NO: 157 - protein, 8 aa, synthetic construct
  SITE 1: note = Optional N-terminal acetylation
  SITE 8: note = Optional C-terminal amidation
PPRPPKRW

SEQ ID NO: 158 - protein, 8 aa, synthetic construct
  SITE 1: note = Optional N-terminal acetylation
  SITE 8: note = Optional C-terminal amidation
PPRPPKRY

SEQ ID NO: 159 - DNA, 38 bp, synthetic construct
  source 1..38: mol_type = other DNA
acctgtacttccagggcatggagatggaaaaggagttc

SEQ ID NO: 160 - DNA, 39 bp, synthetic construct
  source 1..39: mol_type = other DNA
gtgcaggaggagttcgaattctatgtgttgctgttgaac

SEQ ID NO: 161 - DNA, 38 bp, synthetic construct
  source 1..38: mol_type = other DNA
gtgcaggaggagttcgaatttcatttcccattgtgtgg

SEQ ID NO: 162 - DNA, 38 bp, synthetic construct
  source 1..38: mol_type = other DNA
gtgcaggaggagttcgaatttcagtctttatcctcctg

SEQ ID NO: 163 - DNA, 38 bp, synthetic construct
  source 1..38: mol_type = other DNA
gtgcaggaggagttcgaattttaggcaccttcgatcac

SEQ ID NO: 164 - DNA, 44 bp, synthetic construct
  source 1..44: mol_type = other DNA
acctgtacttccagggcatgatgcccgcttatgtgaaatttaac

SEQ ID NO: 165 - DNA, 45 bp, synthetic construct
  source 1..45: mol_type = other DNA
gtgcaggaggagttcgaattctagtcaccttcttcagttacatag

SEQ ID NO: 166 - DNA, 18 bp, synthetic construct
  source 1..18: mol_type = other DNA
tgtcgatcctaccatcca

SEQ ID NO: 167 - DNA, 18 bp, synthetic construct
  source 1..18: mol_type = other DNA
agtcagtcagtgcaggag

SEQ ID NO: 168 - protein, 7 aa, synthetic construct
  SITE 1: note = Pyroglutamic acid
  SITE 7: note = C-terminal amidation
EPRPPKR

SEQ ID NO: 169 - protein, 6 aa, synthetic construct
  SITE 1: note = Pyroglutamic acid
  SITE 6: note = C-terminal amidation
ERPPKR

SEQ ID NO: 170 - DNA, 18 bp, synthetic construct
  source 1..18: mol_type = other DNA
tttttttttttttttttt